SEQUENCE LISTING (8 sequences)

SEQ ID NO: 1 - DNA, 1476 bp, synthetic construct
  source 1..1476: mol_type = other DNA
atgaatccagcagcagaagccgagttcaacatcttattggctacagatagttacaaagtcacccactataagcaataccctccaaatacatctaaagtctattcttacttcgaatgtagagagaagaagactgagaatagtaagttgcgtaaagttaagtatgaggagacagtgttctacggtttacaatacatcttgaacaaatacttgaaaggtaaggtcgtgactaaagagaagattcaggaagcaaaggatgtctacaaagagcacttccaagacgacgtattcaatgagaaaggttggaactacatcttagagaagtatgatggtcatttgccaatcgagatcaaagctgttcctgagggatttgttattccaagaggtaatgtattgtttacagttgagaataccgatccagaatgttattggttaacaaactggattgaaaccatattggttcaatcttggtatcccataactgtagctacaaatagtagggagcagaagaagatattggccaaatacttattggaaacttctggcaatttggacggtttagagtataagttgcacgatttcggttatcgtggtgtatcatctcaagagacagcaggtattggtgcatcagcacatttggtgaacttcaaaggtacagatacagttgctggtttagccttgattaagaagtactatggcactaaggacccagtaccaggctattccgtgccagcagcagaacattcaactatcaccgcttggggaaaggaccatgagaaagatgcattcgaacatatcgtaactcaattcagtagtgttccagtgtctgtggtatcagatagttacgacatctacaatgcatgtgagaagatatggggtgaagatttgaggcatttgattgtttctcgttcaacacaggcacctttgatcatacgtcctgattctggaaatcctttagatactgtgttaaaggtattagaaatcttgggtaagaagttcccagttaccgagaactccaaaggatacaagttgttgccaccttacttgagagtaattcaaggcgatggtgttgacatcaatacattgcaagaaatcgttgaaggtatgaaacagaagatgtggagtatcgagaacattgcatttggcagtggtggaggtttgttgcagaagttgacacgtgatttgttgaattgttcctttaagtgttcttatgtagttaccaatggattaggcatcaacgtcttcaaagatccagttgcagacccaaacaagaggtccaagaaaggacgtttaagtttacaccgtactccagcaggaaactttgttactttagaagaaggtaagggtgatttggaagagtatggacaagatttgttgcataccgtcttcaagaatggaaaggtcaccaaatcctactccttcgacgagatccgtaagaatgctcagttgaacattgaattagaagcagcacatcactaa

SEQ ID NO: 2 - DNA, 837 bp, synthetic construct
  source 1..837: mol_type = other DNA
atggagaactctgagaagaccgaagtagtgttattggcatgtggatcattcaacccaataacaaacatgcatttgagattgtttgaattagctaaagattacatgaacggtactggaaggtacacagttgtaaagggcatcattagtcccgttggtgatgcttacaagaagaagggattgatacctgcctatcaccgtgtcataatggctgaattagcaactaagaactctaagtgggtcgaggtcgatacttgggagagtttacagaaggagtggaaagaaaccttgaaagtattacgtcatcatcaagagaagttggaagcatctgattgtgatcatcagcagaactctcccacattggaaagaccaggaaggaaacgtaagtggacagaaactcaagactcttcacagaagaagagtttggaacctaagacaaaggctgtacccaaagtgaaattgttgtgtggagcagatttgttggaatctttcgctgtacccaatttgtggaagtccgaagacataacacaaattgtggcaaactacggcttgatttgcgtaaccagggctggtaacgacgcacagaagttcatatacgaatcagatgtcttgtggaagcaccgtagtaacattcacgtggtcaacgagtggatagcaaacgacatatcttctactaagatccgtcgtgctttaagaagaggacaatctattaggtacttggttcctgacttggttcaagagtacatcgagaaacacaatttgtattcatccgaatctgaagatagaaatgcaggtgttatcttggcacctttacaaaggaacactgctgaggctaagaca

SEQ ID NO: 3 - DNA, 525 bp, synthetic construct
  source 1..525: mol_type = other DNA
atgaagaccttcatcataggtatttctggtgtcacaaactctggtaagacaacattggccaagaatttgcagaaacatttgccaaactgttcagtaatctcccaggatgatttcttcaagccagagtccgagattgaaacagataagaacggtttcttacagtatgacgtcttggaggctttgaatatggagaagatgatgagtgccattagttgttggatggaatccgcaagacattctgtggtatccaccgatcaagaatctgctgaggaaatacccatcttaatcatagaaggtttcttgttgttcaattacaataccagagtttatcagcctcctgactctccaggttacttcgatggtcatgtatggccaatgtacttgaagtataggcaagaaatgcaagacattacctgggaggtcgtttacttagatggtacaaagtcagaagaagacttgttcttacaagtttatgaagacttgattcaggaattagcaaagcagaaatgtttgcaagtcaccgct

SEQ ID NO: 4 - DNA, 708 bp, synthetic construct
  source 1..708: mol_type = other DNA
atgaaattgattgttggtataggtggtatgacaaatggtggtaagactacattaactaatagtttgttgagggctttgcctaactgttgcgtgattcaccaagacgacttcttcaaagctccattgtttcagcctcaagaccaaattgctgtgggagaggatggtttcaaacaatgggatgtattggaaagtttggatatggaagccatgttggatactgttcaggcttggttgtctagtcctcagaagttcgcaagagcccatggtgtctctgtacaaccagaagcctcagacactcatatcttgttgttggagggattcttattgtattcttacaaacctttagttgacttgtattccagaagatacttcttgacagtaccatacgaagagtgtaagtggcgtagatccacaaggaactacactgttcctgatccaccaggtttgttcgacggtcatgtttggcctatgtatcagaagtacagacaggaaatggaagcaaacggtgttgaggttgtttacttggacggtatgaaatctagggaagaattgtttcgtgaagtcttagaagacatccagaactcattgttaaaccgtagtcaagaaagtgctccttcaccagctagacctgctagaactcagggacctggtcgtggttgtggccacagaactgcaaggccagcagctagtcaacaagattcaatgtaa

SEQ ID NO: 5 - DNA, 891 bp, synthetic construct
  source 1..891: mol_type = other DNA
atggatgccgaaggcttggctttgttattgccacctgtcacattggcagcattagtcgattcatggttgcgtgaggactgcccaggattgaactatgctgcattagtttcaggagctggtccttcacaagctgcattgtgggctaagtcacctggcgtattggcaggtcagccattcttcgatgccatattcacacagttgaattgtcaggtttcttggttcttgcctgagggctccaagttagttccagttgctagagtagctgaagttagaggaccagctcattgtttgttgttgggagaaagagtagcattgaatactttggctaggtgctcaggaatcgccagtgctgctgctgctgctgttgaagctgctagaggagctggctggacaggacacgttgctggcaccaggaagacaacaccaggtttccgtttggttgagaagtatggcttattggttggtggtgctgcctcccacagatacgacttgggtggcttggtaatggttaaagacaaccatgtagtggcagcaggtggtgtggagaaagccgtcagggcagctagacaagcagcagatttcacattgaaagtcgaagtggaatgttcatcattgcaagaggcagttcaagcagcagaggctggtgccgacttggtattgttggataactttaagccagaagaattacacccaactgctactgtcttgaaagcacaatttccatccgtggctgtagaggcttccggtggaattacattggacaacttaccacagttctgcggtccacatattgacgttatctcaatgggtatgttgactcaagcagctccagccttagatttctcattgaagttattcgcaaaggaagtagcaccagtccctaagatccac

SEQ ID NO: 6 - DNA, 13054 bp, synthetic construct
  source 1..13054: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatgaatccagcagcagaagccgagttcaacatcttattggctacagatagttacaaagtcacccactataagcaataccctccaaatacatctaaagtctattcttacttcgaatgtagagagaagaagactgagaatagtaagttgcgtaaagttaagtatgaggagacagtgttctacggtttacaatacatcttgaacaaatacttgaaaggtaaggtcgtgactaaagagaagattcaggaagcaaaggatgtctacaaagagcacttccaagacgacgtattcaatgagaaaggttggaactacatcttagagaagtatgatggtcatttgccaatcgagatcaaagctgttcctgagggatttgttattccaagaggtaatgtattgtttacagttgagaataccgatccagaatgttattggttaacaaactggattgaaaccatattggttcaatcttggtatcccataactgtagctacaaatagtagggagcagaagaagatattggccaaatacttattggaaacttctggcaatttggacggtttagagtataagttgcacgatttcggttatcgtggtgtatcatctcaagagacagcaggtattggtgcatcagcacatttggtgaacttcaaaggtacagatacagttgctggtttagccttgattaagaagtactatggcactaaggacccagtaccaggctattccgtgccagcagcagaacattcaactatcaccgcttggggaaaggaccatgagaaagatgcattcgaacatatcgtaactcaattcagtagtgttccagtgtctgtggtatcagatagttacgacatctacaatgcatgtgagaagatatggggtgaagatttgaggcatttgattgtttctcgttcaacacaggcacctttgatcatacgtcctgattctggaaatcctttagatactgtgttaaaggtattagaaatcttgggtaagaagttcccagttaccgagaactccaaaggatacaagttgttgccaccttacttgagagtaattcaaggcgatggtgttgacatcaatacattgcaagaaatcgttgaaggtatgaaacagaagatgtggagtatcgagaacattgcatttggcagtggtggaggtttgttgcagaagttgacacgtgatttgttgaattgttcctttaagtgttcttatgtagttaccaatggattaggcatcaacgtcttcaaagatccagttgcagacccaaacaagaggtccaagaaaggacgtttaagtttacaccgtactccagcaggaaactttgttactttagaagaaggtaagggtgatttggaagagtatggacaagatttgttgcataccgtcttcaagaatggaaaggtcaccaaatcctactccttcgacgagatccgtaagaatgctcagttgaacattgaattagaagcagcacatcactaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatggagaactctgagaagaccgaagtagtgttattggcatgtggatcattcaacccaataacaaacatgcatttgagattgtttgaattagctaaagattacatgaacggtactggaaggtacacagttgtaaagggcatcattagtcccgttggtgatgcttacaagaagaagggattgatacctgcctatcaccgtgtcataatggctgaattagcaactaagaactctaagtgggtcgaggtcgatacttgggagagtttacagaaggagtggaaagaaaccttgaaagtattacgtcatcatcaagagaagttggaagcatctgattgtgatcatcagcagaactctcccacattggaaagaccaggaaggaaacgtaagtggacagaaactcaagactcttcacagaagaagagtttggaacctaagacaaaggctgtacccaaagtgaaattgttgtgtggagcagatttgttggaatctttcgctgtacccaatttgtggaagtccgaagacataacacaaattgtggcaaactacggcttgatttgcgtaaccagggctggtaacgacgcacagaagttcatatacgaatcagatgtcttgtggaagcaccgtagtaacattcacgtggtcaacgagtggatagcaaacgacatatcttctactaagatccgtcgtgctttaagaagaggacaatctattaggtacttggttcctgacttggttcaagagtacatcgagaaacacaatttgtattcatccgaatctgaagatagaaatgcaggtgttatcttggcacctttacaaaggaacactgctgaggctaagacataagcatgctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcgcgccaccatgaagaccttcatcataggtatttctggtgtcacaaactctggtaagacaacattggccaagaatttgcagaaacatttgccaaactgttcagtaatctcccaggatgatttcttcaagccagagtccgagattgaaacagataagaacggtttcttacagtatgacgtcttggaggctttgaatatggagaagatgatgagtgccattagttgttggatggaatccgcaagacattctgtggtatccaccgatcaagaatctgctgaggaaatacccatcttaatcatagaaggtttcttgttgttcaattacaataccagagtttatcagcctcctgactctccaggttacttcgatggtcatgtatggccaatgtacttgaagtataggcaagaaatgcaagacattacctgggaggtcgtttacttagatggtacaaagtcagaagaagacttgttcttacaagtttatgaagacttgattcaggaattagcaaagcagaaatgtttgcaagtcaccgcttaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggatgccgaaggcttggctttgttattgccacctgtcacattggcagcattagtcgattcatggttgcgtgaggactgcccaggattgaactatgctgcattagtttcaggagctggtccttcacaagctgcattgtgggctaagtcacctggcgtattggcaggtcagccattcttcgatgccatattcacacagttgaattgtcaggtttcttggttcttgcctgagggctccaagttagttccagttgctagagtagctgaagttagaggaccagctcattgtttgttgttgggagaaagagtagcattgaatactttggctaggtgctcaggaatcgccagtgctgctgctgctgctgttgaagctgctagaggagctggctggacaggacacgttgctggcaccaggaagacaacaccaggtttccgtttggttgagaagtatggcttattggttggtggtgctgcctcccacagatacgacttgggtggcttggtaatggttaaagacaaccatgtagtggcagcaggtggtgtggagaaagccgtcagggcagctagacaagcagcagatttcacattgaaagtcgaagtggaatgttcatcattgcaagaggcagttcaagcagcagaggctggtgccgacttggtattgttggataactttaagccagaagaattacacccaactgctactgtcttgaaagcacaatttccatccgtggctgtagaggcttccggtggaattacattggacaacttaccacagttctgcggtccacatattgacgttatctcaatgggtatgttgactcaagcagctccagccttagatttctcattgaagttattcgcaaaggaagtagcaccagtccctaagatccactaacctaggtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggtcgacgccaccatgaaattgattgttggtataggtggtatgacaaatggtggtaagactacattaactaatagtttgttgagggctttgcctaactgttgcgtgattcaccaagacgacttcttcaaagctccattgtttcagcctcaagaccaaattgctgtgggagaggatggtttcaaacaatgggatgtattggaaagtttggatatggaagccatgttggatactgttcaggcttggttgtctagtcctcagaagttcgcaagagcccatggtgtctctgtacaaccagaagcctcagacactcatatcttgttgttggagggattcttattgtattcttacaaacctttagttgacttgtattccagaagatacttcttgacagtaccatacgaagagtgtaagtggcgtagatccacaaggaactacactgttcctgatccaccaggtttgttcgacggtcatgtttggcctatgtatcagaagtacagacaggaaatggaagcaaacggtgttgaggttgtttacttggacggtatgaaatctagggaagaattgtttcgtgaagtcttagaagacatccagaactcattgttaaaccgtagtcaagaaagtgctccttcaccagctagacctgctagaactcagggacctggtcgtggttgtggccacagaactgcaaggccagcagctagtcaacaagattcaatgtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 7 - DNA, 890 bp, synthetic construct
  source 1..890: mol_type = other DNA
ggattcaaagaggagaaatactagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaagctt

SEQ ID NO: 8 - DNA, 717 bp, synthetic construct
  source 1..717: mol_type = other DNA
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

(19) United States
(12) Patent Application Publication
(10) Pub. No.: US 2026/0250695 A1
(43) Pub. Date: Aug. 27, 2026

(54) NICOTINAMIDE ADENINE DINUCLEOTIDE DEVICES AND COMPOSITIONS AND METHODS FOR MAKING THE SAME

(71) Applicant: Bio Capital Holdings, LLC, Houston, TX (US)

(72) Inventor: Raul CUERO RENGIFO, Cypress, TX (US)

(21) Appl. No.: 19/460,654

(22) Filed: Jan. 27, 2026

Related U.S. Application Data
(60) Provisional application No. 63/750,334, filed on Jan. 28, 2025.

(51) Int. Cl. C12N 15/81; C12N 9/10; C12N 9/12

(52) U.S. Cl. CPC C12N 15/81; C12N 9/1077; C12N 9/1205; C12N 9/1241; C12Y 204/02012; C12Y 204/02019; C12Y 207/07001

(57) ABSTRACT

In one aspect, disclosed herein are DNA constructs and biological devices comprising S. cerevisiae cells and extracts and lysates produced therefrom, wherein the DNA constructs encode genes related to NAD+ synthesis and wherein the extracts and lysates produced therefrom include one or more of NAD+, NADP+, NADH, and/or NADPH. Also disclosed are nutritional and pharmaceutical compositions including the extracts and lysates and the coenzymes produced therefrom. In some aspects, the disclosed extracts and lysates can be added to food and beverage products to improve the nutritional profiles of the same.

### CROSS-REFERENCE TO RELATED APPLICATIONS

This application claims the benefit of U.S. Provisional Application No. 63/750,334 filed on Jan. 28, 2025, which is incorporated herein by reference in its entirety.

### CROSS REFERENCE TO SEQUENCE LISTING

The genetic components described herein are referred to by sequence identifier numbers (SEQ ID NO). The sequence listing in written computer readable format (CRF) as an xml file named “930201-1210_Sequence_Listing.xml” created on Jan. 20, 2026, and having a size of 27,131 bytes, is incorporated by reference in its entirety.

### BACKGROUND

Nicotinamide adenine dinucleotide (also referred to as NAD or NAD+) is a coenzyme present in either oxidized or reduced form in the body. NAD+ exists in phosphorylated and non-phosphorylated forms (forming NADP+ or NADPH). NAD+ is important in mitochondria energy production, including the synthesis of adenosine triphosphate (ATP) and has recently been implicated in several anti-aging processes and pathways in the body. Although the body can produce NAD+ from food, levels of NAD+ tend to decline with age, leading to some of the effects associated with aging including mitochondrial dysfunction. NAD(P)/H is further involved in posttranslational modification of proteins with NADP having a particular role in anabolic metabolism, including building proteins and other macromolecules from simpler molecules.

NAD+ is also vital to the function of proteins known as sirtuins, which are highly-conserved enzymes involved in metabolic regulation and which couple lysine deacetylation to NAD+ hydrolysis. Sirtuins have also been implicated in cellular processes related to inflammation, oxidative stress, and cell death. As NAD+ availability decreases with age, sirtuin activities decrease, which may increase cellular aging and death. It may thus be desirable to supplement the diets of aging and chronically ill individuals with NAD+ in order to maintain general health and reduce inflammation, among other benefits.

It would be advantageous to develop new supplemental forms of nutrition that include but are not limited to, NAD+. It would further be advantageous if these nutritional supplements could be absorbed in the oral cavity through digestion by saliva proteases or through sublingual absorption, although other methods of administration including injection and inhalation are contemplated and should also be considered disclosed. It would also be advantageous if these nutritional supplements could be consumed alone or could be added to other food and beverage products. It would further be advantageous if the nutritional compounds in these nutritional supplements could be produced inexpensively and quickly, in high volumes. The present invention addresses these needs.

### SUMMARY

In one aspect, disclosed herein are DNA constructs and biological devices comprising S. cerevisiae cells and extracts and lysates produced therefrom, wherein the DNA constructs encode genes related to NAD+ synthesis and wherein the extracts and lysates produced therefrom include one or more of NAD+, NADP+, NADH, and/or NADPH. Also disclosed are nutritional and pharmaceutical compositions including the extracts and lysates and the coenzymes produced therefrom. The extracts and lysates from these devices can be used alone in oral dosage forms for nutritional supplementation or can be used in combination with other lysates and extracts containing vitamins such as carotenoids, organic electrolytes, flavorants such as steviol glycosides, and the like. Also disclosed herein are oral dosage forms including sublingual and buccal dosage forms comprising the disclosed extracts and lysates. In some aspects, the disclosed lysates and extracts can be encapsulated in hydrogel microparticles in order to facilitate absorption. In some aspects, the disclosed extracts and lysates can be added to food and beverage products to improve the nutritional profiles of the same.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

Many aspects of the present disclosure can be better understood with reference to the following drawings, which are incorporated in and constitute a part of this specification. The components in the drawings are not necessarily to scale, emphasis instead being placed upon clearly illustrating the principles of the present disclosure.

FIGS. 1A-1B show, respectively, linear and circular maps of an exemplary biological device for producing NAD and related metabolites including nicotinamide phosphoribosyltransferase (NAMPT), nicotinamide mononucleotide adenylyltransferase (NMNAT), nicotinamide riboside kinase 1, nicotinamide riboside kinase 2, and quinolinate phosphoribosyltransferase (QPRT).

FIG. 2 shows a standard curve for the solubility of an exemplary disclosed lysate. This figure shows a linear straight curve passing through or touching each of the concentration points, indicating total solubility in water of the disclosed lysate.

FIG. 3 shows a standard curve for the solubility of a control (commercial NAD). This figure shows the best fit curve does not touch or pass through any of the concentration points, which indicates that the commercial NAD was not totally dissolved in water.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are biological devices and methods to produce nicotinamide adenine dinucleotide (NAD) in its phosphorylated and non-phosphorylated forms. In a further aspect, the nicotinamide adenine dinucleotide can be oxidized or reduced (e.g., NAD+, NADH, NADP+, NADPH). In a further aspect, NAD is known to be produced in vivo by a de novo pathway and a salvage pathway, and the disclosed devices and methods make use of enzymes involved in both the de novo pathway and the salvage pathway, thereby increasing the total yield of NAD from the disclosed devices. Also disclosed is an NAD composition produced by the disclosed devices and methods. In one aspect, lysates and extracts from the devices can be included in nutritional compositions also containing proteins as disclosed herein. In another aspect, the NAD composition can be administered alone or separately from protein-containing lysates and extracts.

Disclosed herein are DNA constructs containing the following genetic components:
(a) a gene that encodes nicotinamide phosphoribosytransferase (NAMPT);
(b) a gene that encodes nicotinamide mononucleotide adenylyltransferase (NMNAT);
(c) a gene that encodes nicotinamide riboside kinase 1 (NRK1);
(d) a gene that encodes nicotinamide riboside kinase 2 (NRK2); and
(e) a gene that encodes quinolinate phosphoribosyltransferase (QPRT).

The DNA constructs may variously encode genes encoding reporter proteins, genes encoding resistance to one or more antibiotics, and the like, and may include regulatory sequences including promoters, terminators, ribosomal binding sites, LAC operons, or other components necessary for the replication of and expression of the genes encoded by the DNA constructs inside microbial hosts such as, for example, Saccharomyces cerevisiae, Escherichia coli, and other microorganisms, including beneficial bacterial cells, such as Lactobacillus species, as well as through stem cell cultures. Also disclosed are vectors including the DNA constructs and biological devices consisting of host cells that include one or more copies of the vectors. In some aspects, the DNA constructs can include salivary enzymes such as, for example, cathepsins, serine proteases, peptidases, and combinations thereof.

Described herein are microbial cultures and extracts containing various forms of nicotinamide adenine dinucleotide including NAD, NADH, NAD(P), and/or NAD(P)H, and methods of making and using thereof. In one aspect, the method of making a disclosed culture or extract includes the steps of (a) making a DNA construct containing genes for producing a NAMPT, NMNAT, NRK1, NRK2, and QPRT, (b) introducing the DNA construct into host microbial cells via transformation or transfection, and (c) culturing the microbial cells to produce the NAD and forms thereof. The cultures are grown in standard media for host cells such as, for example, S. cerevisiae. The compositions of these cultures and extracts can be tailored to have specific properties such as, for example, the ability to provide nutrition in the form of protein, vitamins, and/or minerals to patients in need thereof.

Before the present compounds, compositions, articles, devices, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In this specification and in the claims that follow, reference will be made to a number of terms that shall defined to have the following meanings:

It must be noted that, as used in the specification and the appended claims, the singular forms “a,” “an,” and “the” include plural referents unless the context clearly dictates otherwise. Thus, for example, reference to “a metabolite” includes mixtures of two or more such metabolites, and the like.

“Optional” or “optionally” means that the subsequently described event or circumstance can or cannot occur, and that the description includes instances where the event or circumstance occurs and instances where it does not. For example, the phrase “a microorganism is optionally genetically modified” means that the microorganism may or may not be genetically modified.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

As used herein, the term “about” is used to provide flexibility to a numerical range endpoint by providing that a given numerical value may be “a little above” or “a little below” the endpoint without affecting the desired result. For purposes of the parent disclosure, “about” refers to a range extending from 10% below the numerical value to 10% above the numerical value. For example, if the numerical value is 10, “about 10” means between 9 and 11, inclusive of the endpoints 9 and 11.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed that while specific reference to each various individual and collective combination and permutation of these compounds may not be explicitly disclosed, each is specifically contemplated and described herein. For example, if a fungal species is disclosed and discussed and a number of different culture media are discussed, each and every combination and permutation of fungus and additional culture medium that is possible is specifically contemplated unless specifically indicated to the contrary. For example, if a class of molecules A, B, and C are disclosed as well as a class of molecules D, E, and F, and an example of a combination molecule, A-D, is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the subgroup of A-E, B-F, and C-E is specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed, it is understood that each of these additional steps can be performed with any specific embodiment or combination of elements of the disclosed methods, and that each such combination is specifically contemplated and should be considered disclosed.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

“Carotenoids” as used herein are red, yellow, and orange hydrophobic pigments. Carotenoids are important plant metabolites from numerous agricultural and other crops including, but not limited to, tomatoes, carrots, and watermelon. In one aspect, the processes disclosed herein are useful for increasing the production of carotenoids from plants, plant calluses, and/or microorganisms engineered to produce carotenoids. In one aspect, carotenoids can be useful in numerous aspects of human health including maintaining vision and fighting cancer. Lycopene is one example of a carotenoid. In one aspect, the biological devices disclosed in U.S. Pat. No. 9,828,609 and international patent application publication WO2019/055326 can be used to produce carotenoids including, but not limited to, lycopene.

“Nicotinamide adenine dinucleotide” or “NAD” is a coenzyme. In some aspects, NAD may be phosphorylated to form NADP. Through redox reactions, NAD switches from an oxidized (NAD(P)+) to a reduced (NAD(P)H) form and back, and is thus a key component of many electron transfer reactions in the body. In one aspect, NAD is important to many metabolic reactions including, but not limited to, production of lycopene, in post-translational modification of proteins, in protein production, and the like. In one aspect, the nutritional compositions disclosed herein include NAD or NADP. Further in this aspect, the NAD(P) can enhance absorption of proteins or other molecules in the compositions, or can act as a carrier for the same. In an alternative aspect, the NAD(P) can be taken separately to enhance the effects of the nutritional compositions. NAD(P) is produced by a de novo pathway as well as a salvage pathway and the disclosed DNA constructs and biological devices can produce NAD(P) by one or both of the de novo and/or salvage pathways.

### DNA Constructs and Biological Devices

In one aspect, cells transformed with a DNA construct can be used in the methods described herein. It is understood that one way to define the variants and derivatives of the genetic components and DNA constructs described herein is in terms of homology/identity to specific known sequences. Those of skill in the art readily understand how to determine the homology of two nucleic acids. For example, the homology can be calculated after aligning two sequences so that the homology is at its highest level. Another way of calculating homology can be performed according to published algorithms (see Zuker, M., Science, 244:48-52, 1989; Jaeger et al, Proc. Natl. Acad. Sci. USA, 86:7706-7710, 1989; Jaeger et al, Methods Enzymol., 183:281-306, 1989, which are herein incorporated by reference for at least material related to nucleic acid alignment).

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of mutations and homology can be combined together in any combination, such as embodiments that have at least about 70%, about 75%, about 80%, about 85%, about 90%, about 95%, or about 99% homology to a particular sequence wherein the variants are conservative or silent mutations. It is understood that any of the sequences described herein can be a variant or derivative having the homology values listed above.

In some aspects, genes of interest can be spliced into suitable vectors using restriction enzymes and/or other techniques known in the art. Further in this aspect, synthesis and/or isolation of the genes of interest prior to inclusion in the disclosed constructs may result in the addition of C-terminal and/or N-terminal sequence data including, but not limited to, restriction enzyme recognition sites, linking bases, short segments of chromosomal DNA (including introns or portions of introns if the sequences originate from eukaryotic cells), transposons, nucleotide repeats, regulatory sequences, and/or other material that do not contribute to the known structure of the expressed protein, or are not part of the expressed protein's active site. In one aspect, presence of these remnants may lead to somewhat reduced homology with respect to gene sequence, but the DNA constructs encoding the same can still produce proteins having the desired sequence, active site, and function.

In another aspect, many eukaryotic genes include introns and mRNAs produced during transcription of the same can be spliced differently, producing several transcript variants from the same gene but having slightly different sequences (i.e., reduced levels of homology). In one aspect, different transcript variants can produce proteins having the same active site but differing in another way (e.g. in C-terminal or N-terminal sequence, affecting assembly of protein subunits or other folding processes, cellular localization of the peptides or proteins, or activity level of the peptides or proteins produced due to differential regulation, or the like.

In one aspect, a database such as, for example, GenBank, can be used to determine the sequences of genes and/or regulatory regions of interest, the species from which these elements originate, and related homologous sequences.

In one aspect, the nucleic acids used in the DNA constructs described herein can be amplified using polymerase chain reaction (PCR) prior to being ligated into a plasmid or other vector. Typically, PCR-amplification techniques make use of primers, or short, chemically-synthesized oligonucleotides that are complementary to regions on each respective strand flanking the DNA or nucleotide sequence to be amplified. A person having ordinary skill in the art will be able to design or choose primers based on the desired experimental conditions. In general, primers should be designed to provide for both efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the skilled practitioner with primer design. Primers can optionally incorporate restriction enzyme recognition sites at their 5′ ends to assist in later ligation into plasmids or other vectors.

PCR can be carried out using purified DNA, unpurified DNA that is integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase, such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or genes. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In a further aspect, the plasmid can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of that coding sequence.

As used herein, “expression” refers to transcription and/or accumulation of an mRNA derived from a gene or DNA fragment. Expression may also be used to refer to translation of mRNA into a peptide, polypeptide, or protein.

### NAD-Producing DNA Constructs

In one aspect, provided herein are DNA constructs having at least the following genetic components:
(a) a gene that encodes nicotinamide phosphoribosyltransferase (NAMPT);
(b) a gene that encodes nicotinamide mononucleotide adenylyltransferase (NMNAT);
(c) a gene that encodes nicotinamide riboside kinase 1 (NRK1);
(d) a gene that encodes nicotinamide riboside kinase 2 (NRK2); and
(e) a gene that encodes quinolinate phosphoribosytransferase (QPRT).

Each component of the DNA constructs is described in detail below. The components can be present in any order.

In one aspect, the DNA constructs disclosed herein incorporate a gene that encodes nicotinamide phosphoribosyltransferase (NAMPT). In a further aspect, NAMPT is a rate-limiting enzyme in the salvage pathway for producing NAD+; NAMPT converts nicotinamide to nicotinamide mononucleotide. In some cases, NAMPT can also synthesize NMN from phosphoribosyl pyrophosphate in the presence of ATP. NAMPT can be found in both intracellular and extracellular forms.

In one aspect, the gene that encodes NAMPT is isolated from a mammal. In a further aspect, the gene that encodes NAMPT has SEQ ID NO. 1 or at least 70% homology thereto, at least 75% homology thereto, at least 80% homology thereto, at least 85% homology thereto, at least 90% homology thereto, at least 95% homology thereto, or at least 99% homology thereto.

Other sequences encoding NAMPT or related or homologous genes can be identified in a database such as, for example, GenBank. In one aspect, the gene that encodes NAMPT is isolated from a human or human cDNA and can be identified by the GI number KJ898163.1 in the GenBank database. In another aspect, sequences useful herein include those with GI numbers listed in Table 1:

TABLE 1
NAMPT

| Source Organism | Sequence Description | GI Number |
|---|---|---|
| synthetic construct | Nicotinamide phosphoribosyltransferase (NAMPT) | KJ898163.1 |
| Pan paniscus | Nicotinamide phosphoribosyltransferase (NAMPT) | XM_034964750.3 |
| Homo sapiens | Nicotinamide phosphoribosyltransferase (NAMPT) | XM_054356984.1 |
| synthetic construct | Nicotinamide phosphoribosyltransferase (NAMPT) | LT742263.1 |
| synthetic construct | Pre-B-cell colony-enhancing factor (PBEF1) | HQ447548.1 |
| Homo sapiens | Nicotinamide phosphoribosyltransferase (NAMPT) | NM_005746.3 |
| Homo sapiens | Nicotinamide phosphoribosyltransferase (NAMPT) | BC106046.1 |
| Homo sapiens | Nicotinamide phosphoribosyltransferase (NAMPT) | BC072439.1 |
| Pan troglodytes | Nicotinamide phosphoribosyltransferase (NAMPT) | XM_016945642.4 |
| Homo sapiens | Nicotinamide phosphoribosyltransferase (NAMPT) | XM_047419699.1 |
| Homo sapiens | Pre-B-cell colony-enhancing factor (PBEF1) | AK292851.1 |
| Pan troglodytes | Nicotinamide phosphoribosyltransferase (NAMPT) | XM_016945641.3 |
| Homo sapiens | Pre-B-cell colony-enhancing factor (PBEF1) | U02020.1 |
| Pan paniscus | Nicotinamide phosphoribosyltransferase (NAMPT) | XM_034964752.2 |
| synthetic construct | Pre-B-cell colony-enhancing factor (PBEF1) | AB590725.1 |
| Gorilla gorilla gorilla | Nicotinamide phosphoribosyltransferase (NAMPT) | XM_004046013.5 |
| Hylobates moloch | Nicotinamide phosphoribosyltransferase (NAMPT) | XM_032761351.2 |
| Symphalangus syndactylus | Nicotinamide phosphoribosyltransferase (NAMPT) | XM_055283604.2 |
| Orycteropus afer afer | Nicotinamide phosphoribosyltransferase (NAMPT) | XM_007944436.2 |
| Sus scrofa domesticus | Nicotinamide phosphoribosyltransferase (NAMPT) | MW052047.1 |
| Dasypus novemcinctus | Nicotinamide phosphoribosyltransferase (NAMPT) | XM_058297148.1 |
| Hippopotamus amphibius kiboko | Nicotinamide phosphoribosyltransferase (NAMPT) | XM_057731415.1 |
| Suncus etruscus | Nicotinamide phosphoribosyltransferase (NAMPT) | XM_049783309.1 |
| Dasypus novemcinctus | Nicotinamide phosphoribosyltransferase (NAMPT) | XM_058297147.1 |
| Kogia breviceps | Nicotinamide phosphoribosyltransferase (NAMPT) | XM_059074539.2 |
| Kogia breviceps | Nicotinamide phosphoribosyltransferase (NAMPT) | XR_010842287.1 |
| Nomascus leucogenys | Nicotinamide phosphoribosyltransferase (NAMPT) | XM_003268147.3 |
| Sorex fumeus | Nicotinamide phosphoribosyltransferase (NAMPT) | XM_056118272.1 |
| Kogia breviceps | Nicotinamide phosphoribosyltransferase (NAMPT) | XR_009337401.2 |
| Kogia breviceps | Nicotinamide phosphoribosyltransferase (NAMPT) | XR_010842288.1 |
| Pongo abelii | Nicotinamide phosphoribosyltransferase (NAMPT) | XM_009235897.4 |
| Neophocaena asiaeorientalis asiaeorientalis | Nicotinamide phosphoribosyltransferase (NAMPT) | XM_024760867.1 |
| Monodon monoceros | Nicotinamide phosphoribosyltransferase (NAMPT) | XM_029233596.1 |
| Phocoena sinus | Nicotinamide phosphoribosyltransferase (NAMPT) | XM_032642788.1 |
| Phocoena sinus | Nicotinamide phosphoribosyltransferase (NAMPT) | XR_004351397.1 |
| Sus scrofa | Pre-B-cell colony-enhancing factor (PBEF1) | EF537042.1 |
| Pongo pygmaeus | Nicotinamide phosphoribosyltransferase (NAMPT) | XM_054495668.2 |
| Sus scrofa | Nicotinamide phosphoribosyltransferase (NAMPT) | NM_001031793.2 |
| Pongo pygmaeus | Nicotinamide phosphoribosyltransferase (NAMPT) | XM_063668432.1 |
| Sus scrofa | Pre-B-cell colony-enhancing factor (PBEF1) | DQ001974.2 |
| Lemur catta | Nicotinamide phosphoribosyltransferase (NAMPT) | XM_045565463.1 |
| Dipodomys spectabilis | Nicotinamide phosphoribosyltransferase (NAMPT) | XM_042684546.1 |
| Perognathus longimembris pacificus | Nicotinamide phosphoribosyltransferase (NAMPT) | XM_048339818.1 |
| Hipposideros armiger | Nicotinamide phosphoribosyltransferase (NAMPT) | XM_019660160.1 |
| Rhinolophus sinicus | Nicotinamide phosphoribosyltransferase (NAMPT) | XM_019745377.1 |
| Lipotes vexillifer | Nicotinamide phosphoribosyltransferase (NAMPT) | XM_007453335.1 |
| Phacochoerus africanus | Nicotinamide phosphoribosyltransferase (NAMPT) | XM_047751888.1 |
| Sus scrofa | Pre-B-cell colony-enhancing factor (PBEF1) | EU127840.1 |
| Microcebus murinus | Nicotinamide phosphoribosyltransferase (NAMPT) | XM_012746431.1 |
| Tursiops truncatus | Nicotinamide phosphoribosyltransferase (NAMPT) | XM_033862896.1 |
| Phocoena phocoena | Nicotinamide phosphoribosyltransferase (NAMPT) | XM_065883389.1 |
| Orcinus orca | Nicotinamide phosphoribosyltransferase (NAMPT) | XM_033439092.2 |
| Delphinapterus leucas | Nicotinamide phosphoribosyltransferase (NAMPT) | XM_022554333.2 |
| Lagenorhynchus albirostris | Nicotinamide phosphoribosyltransferase (NAMPT) | XM_060157274.1 |
| Orcinus orca | Nicotinamide phosphoribosyltransferase (NAMPT) | XM_004263362.3 |
| Globicephala melas | Nicotinamide phosphoribosyltransferase (NAMPT) | XM_030834390.3 |
| Delphinapterus leucas | Nicotinamide phosphoribosyltransferase (NAMPT) | XR_002642121.2 |
| Orcinus orca | Nicotinamide phosphoribosyltransferase (NAMPT) | XM_033439101.2 |
| Pseudorca crassidens | Nicotinamide phosphoribosyltransferase (NAMPT) | XM_067745958.1 |
| Globicephala melas | Nicotinamide phosphoribosyltransferase (NAMPT) | XR_009565130.2 |
| Tursiops truncatus | Nicotinamide phosphoribosyltransferase (NAMPT) | XM_033862897.1 |
| Delphinapterus leucas | Nicotinamide phosphoribosyltransferase (NAMPT) | XR_002642120.2 |
| Dipodomys merriami | Nicotinamide phosphoribosyltransferase (NAMPT) | XM_069996248.1 |
| Galeopterus variegatus | Nicotinamide phosphoribosyltransferase (NAMPT) | XM_008567863.1 |
| Galeopterus variegatus | Nicotinamide phosphoribosyltransferase (NAMPT) | XM_008567870.1 |
| Chrysochloris asiatica | Nicotinamide phosphoribosyltransferase (NAMPT) | XM_006834384.1 |
| Lagenorhynchus obliquidens | Nicotinamide phosphoribosyltransferase (NAMPT) | XM_027123892.1 |
| Lagenorhynchus obliquidens | Nicotinamide phosphoribosyltransferase (NAMPT) | XM_027123891.1 |
| Odocoileus virginianus | Nicotinamide phosphoribosyltransferase (NAMPT) | XM_020893508.2 |
| Capra hircus | Nicotinamide phosphoribosyltransferase (NAMPT) | XM_018047232.1 |
| Capricornis sumatraensis | Nicotinamide phosphoribosyltransferase (NAMPT) | XM_068972590.1 |
| Budorcas taxicolor | Nicotinamide phosphoribosyltransferase (NAMPT) | XM_052638503.1 |
| Rhinopithecus roxellana | Nicotinamide phosphoribosyltransferase (NAMPT) | XM_010380669.2 |
| Balaenoptera ricei | Nicotinamide phosphoribosyltransferase (NAMPT) | XM_059934165.1 |
| Sus scrofa | Nicotinamide phosphoribosyltransferase (NAMPT) | EU707339.1 |
| Balaenoptera ricei | Nicotinamide phosphoribosyltransferase (NAMPT) | XM_059934164.1 |
| Capra hircus | Nicotinamide phosphoribosyltransferase (NAMPT) | KY996482.1 |
| Mesoplodon densirostris | Nicotinamide phosphoribosyltransferase (NAMPT) | XM_060108556.1 |
| Trachypithecus francoisi | Nicotinamide phosphoribosyltransferase (NAMPT) | XM_033194871.1 |
| Delphinus delphis | Nicotinamide phosphoribosyltransferase (NAMPT) | XR_009520592.1 |
| Delphinus delphis | Nicotinamide phosphoribosyltransferase (NAMPT) | XM_060020264.1 |
| Sus scrofa | Genomic DNA | AK233280.1 |
| Trichechus manatus latirostris | Nicotinamide phosphoribosyltransferase (NAMPT) | XM_004382605.3 |
| Eschrichtius robustus | Nicotinamide phosphoribosyltransferase (NAMPT) | XM_068550392.1 |
| Ovis aries | Nicotinamide phosphoribosyltransferase (NAMPT) | XM_004007842.5 |
| Myotis lucifugus | Nicotinamide phosphoribosyltransferase (NAMPT) | XM_006085386.3 |
| Pteropus vampyrus | Nicotinamide phosphoribosyltransferase (NAMPT) | XM_039849466.1 |
| Choloepus didactylus | Nicotinamide phosphoribosyltransferase (NAMPT) | XM_037836405.1 |
| Choloepus didactylus | Nicotinamide phosphoribosyltransferase (NAMPT) | XM_037836406.1 |
| Bubalus kerabau | Nicotinamide phosphoribosyltransferase (NAMPT) | XM_055590234.1 |
| Bubalus kerabau | Nicotinamide phosphoribosyltransferase (NAMPT) | XM_055590232.1 |
| Bubalus kerabau | Nicotinamide phosphoribosyltransferase (NAMPT) | XM_055590233.1 |
| Bubalus kerabau | Nicotinamide phosphoribosyltransferase (NAMPT) | XM_055590235.1 |
| Bubalus kerabau | Nicotinamide phosphoribosyltransferase (NAMPT) | XM_055590229.1 |
| Ovis canadensis | Nicotinamide phosphoribosyltransferase (NAMPT) | XM_069587890.1 |
| Bubalus kerabau | Nicotinamide phosphoribosyltransferase (NAMPT) | XM_055590237.1 |
| Bubalus kerabau | Nicotinamide phosphoribosyltransferase (NAMPT) | XM_055590230.1 |
| Eulemur rufifrons | Nicotinamide phosphoribosyltransferase (NAMPT) | XM_069462558.1 |
| Cynocephalus volans | Nicotinamide phosphoribosyltransferase (NAMPT) | XM_063100127.1 |
| Physeter macrocephalus | Nicotinamide phosphoribosyltransferase (NAMPT) | XM_024115955.2 |

In one aspect, the DNA constructs disclosed herein incorporate a gene that encodes nicotinamide mononucleotide adenylyltransferase (NMNAT). In a further aspect, NMNAT catalyzes the formation of NAD+ from ATP and nicotinamide mononucleotide. Different NMNAT enzyme isoforms are expressed in the nucleus, cytoplasm, and mitochondria and typically function by reversibly catalyzing the transfer of the adenosyl part of ATP to nicotinamide mononucleotide. NMNAT is typically considered to be involved in a salvage pathway of NAD synthesis.

In one aspect, the gene that encodes NMNAT is isolated from a mammal. In a further aspect, the gene that encodes NMNAT has SEQ ID NO. 2 or at least 70% homology thereto, at least 75% homology thereto, at least 80% homology thereto, at least 85% homology thereto, at least 90% homology thereto, at least 95% homology thereto, or at least 99% homology thereto.

Other sequences encoding NMNAT or related or homologous genes can be identified in a database such as, for example, GenBank. In one aspect, the gene that encodes NMNAT is isolated from a human and can be identified by the GI number AF312734.1 in the GenBank database. In another aspect, sequences useful herein include those with GI numbers listed in Table 2:

TABLE 2
NMNAT

| Source | | |
|---|---|---|
| Organism | Sequence Description | GI Number |
| Homo sapiens | Nicotinamide nucleotide adenylyltransferase (NMNAT) | AF312734.1 |
| Homo sapiens | Nicotinamide nucleotide adenylyltransferase (NMNAT) | XM_017002107.3 |
| Homo sapiens | Nicotinamide nucleotide adenylyltransferase (NMNAT) | LT737168.1 |
| Homo sapiens | Nicotinamide nucleotide adenylyltransferase (NMNAT) | BC014943.1 |
| Homo sapiens | Nicotinamide nucleotide adenylyltransferase (NMNAT) | XM_054338308.1 |
| Homo sapiens | Nicotinamide nucleotide adenylyltransferase (NMNAT) | XM_047428076.1 |
| Homo sapiens | Nicotinamide nucleotide adenylyltransferase (NMNAT) | NM_022787.4 |
| Homo sapiens | Nicotinamide nucleotide adenylyltransferase (NMNAT) | AF314163.1 |
| synthetic construct | Nicotinamide nucleotide adenylyltransferase (NMNAT) | KJ894578.1 |
| Homo sapiens | Nicotinamide nucleotide adenylyltransferase (NMNAT) | XM_054338309.1 |
| Homo sapiens | Nicotinamide nucleotide adenylyltransferase (NMNAT) | NM_001297778.1 |
| synthetic construct | Nicotinamide nucleotide adenylyltransferase (NMNAT) | JF432503.1 |
| Pan troglodytes | Nicotinamide nucleotide adenylyltransferase (NMNAT) | XR_010155026.1 |
| Gorilla gorilla gorilla | Nicotinamide nucleotide adenylyltransferase (NMNAT) | XM_063702861.1 |
| Homo sapiens | Genomic DNA | AK026065.1 |
| Gorilla gorilla gorilla | Nicotinamide nucleotide adenylyltransferase (NMNAT) | XR_008678748.2 |
| Gorilla gorilla gorilla | Nicotinamide nucleotide adenylyltransferase (NMNAT) | XR_010132472.1 |
| Pan paniscus | Nicotinamide nucleotide adenylyltransferase (NMNAT) | XM_055104131.2 |
| Gorilla gorilla gorilla | Nicotinamide nucleotide adenylyltransferase (NMNAT) | XR_010132474.1 |
| Homo sapiens | Nicotinamide nucleotide adenylyltransferase (NMNAT) | AK222920.1 |
| Pan troglodytes | Nicotinamide nucleotide adenylyltransferase (NMNAT) | XM_054662651.2 |
| Pan paniscus | Nicotinamide nucleotide adenylyltransferase (NMNAT) | XM_055104129.2 |
| Homo sapiens | Nicotinamide nucleotide adenylyltransferase (NMNAT) | AF459819.1 |
| Pan paniscus | Nicotinamide nucleotide adenylyltransferase (NMNAT) | XM_034954984.3 |
| Pan troglodytes | Nicotinamide nucleotide adenylyltransferase (NMNAT) | XM_016953779.4 |
| Gorilla gorilla gorilla | Nicotinamide nucleotide adenylyltransferase (NMNAT) | XM_019010790.3 |
| Homo sapiens | Nicotinamide nucleotide adenylyltransferase (NMNAT) | AK315640.1 |
| Pan paniscus | Nicotinamide nucleotide adenylyltransferase (NMNAT) | XM_055104127.2 |
| Gorilla gorilla gorilla | Nicotinamide nucleotide adenylyltransferase (NMNAT) | XM_019010795.3 |
| Pan troglodytes | Nicotinamide nucleotide adenylyltransferase (NMNAT) | XM_063805068.1 |
| Pan paniscus | Nicotinamide nucleotide adenylyltransferase (NMNAT) | XM_003822108.5 |
| Pan troglodytes | Nicotinamide nucleotide adenylyltransferase (NMNAT) | XM_016953770.4 |
| Pongo pygmaeus | Nicotinamide nucleotide adenylyltransferase (NMNAT) | XM_054440168.2 |
| Nomascus leucogenys | Nicotinamide nucleotide adenylyltransferase (NMNAT) | XM_030805519.1 |
| Symphalangus syndactylus | Nicotinamide nucleotide adenylyltransferase (NMNAT) | XM_063631992.1 |
| Pongo pygmaeus | Nicotinamide nucleotide adenylyltransferase (NMNAT) | XM_054440163.2 |
| Symphalangus syndactylus | Nicotinamide nucleotide adenylyltransferase (NMNAT) | XM_055261891.1 |
| Pongo abelii | Nicotinamide nucleotide adenylyltransferase (NMNAT) | XM_054556703.2 |
| Pongo pygmaeus | Nicotinamide nucleotide adenylyltransferase (NMNAT) | XM_063668088.1 |
| Pongo pygmaeus | Nicotinamide nucleotide adenylyltransferase (NMNAT) | XM_063668093.1 |
| Symphalangus syndactylus | Nicotinamide nucleotide adenylyltransferase (NMNAT) | XM_063631993.1 |
| Nomascus leucogenys | Nicotinamide nucleotide adenylyltransferase (NMNAT) | XM_030805520.1 |
| Pongo pygmaeus | Nicotinamide nucleotide adenylyltransferase (NMNAT) | XM_054440153.2 |
| Pongo pygmaeus | Nicotinamide nucleotide adenylyltransferase (NMNAT) | XM_054440179.2 |
| Pongo pygmaeus | Nicotinamide nucleotide adenylyltransferase (NMNAT) | XM_063668098.1 |
| Pongo abelii | Nicotinamide nucleotide adenylyltransferase (NMNAT) | XM_024231429.3 |
| Pongo pygmaeus | Nicotinamide nucleotide adenylyltransferase (NMNAT) | XM_054440144.2 |
| Symphalangus syndactylus | Nicotinamide nucleotide adenylyltransferase (NMNAT) | XR_010118966.1 |
| Pongo pygmaeus | Nicotinamide nucleotide adenylyltransferase (NMNAT) | XM_063668104.1 |
| Pongo abelii | Nicotinamide nucleotide adenylyltransferase (NMNAT) | XM_063715224.1 |
| Papio anubis | Nicotinamide nucleotide adenylyltransferase (NMNAT) | XM_017957050.3 |
| Papio anubis | Nicotinamide nucleotide adenylyltransferase (NMNAT) | XM_009189659.4 |
| Papio anubis | Nicotinamide nucleotide adenylyltransferase (NMNAT) | XM_003891083.5 |
| Papio anubis | Nicotinamide nucleotide adenylyltransferase (NMNAT) | XM_017957046.3 |
| Papio anubis | Nicotinamide nucleotide adenylyltransferase (NMNAT) | XM_017957042.3 |
| Symphalangus syndactylus | Nicotinamide nucleotide adenylyltransferase (NMNAT) | XM_063631994.1 |
| Hylobates moloch | Nicotinamide nucleotide adenylyltransferase (NMNAT) | XM_032758857.2 |
| Cercocebus atys | Nicotinamide nucleotide adenylyltransferase (NMNAT) | XM_012049423.1 |
| Cercocebus atys | Nicotinamide nucleotide adenylyltransferase (NMNAT) | XM_012049422.1 |
| Cercocebus atys | Nicotinamide nucleotide adenylyltransferase (NMNAT) | XM_012049419.1 |
| Cercocebus atys | Nicotinamide nucleotide adenylyltransferase (NMNAT) | XM_012049418.1 |
| Cercocebus atys | Nicotinamide nucleotide adenylyltransferase (NMNAT) | XM_012049421.1 |
| Macaca fascicularis | Nicotinamide nucleotide adenylyltransferase (NMNAT) | XM_005544841.4 |
| Macaca fascicularis | Nicotinamide nucleotide adenylyltransferase (NMNAT) | XM_005544840.4 |
| Theropithecus gelada | Nicotinamide nucleotide adenylyltransferase (NMNAT) | XM_025404454.1 |
| Theropithecus gelada | Nicotinamide nucleotide adenylyltransferase (NMNAT) | XM_025404445.1 |
| Macaca fascicularis | Nicotinamide nucleotide adenylyltransferase (NMNAT) | XM_045381017.2 |
| Macaca fascicularis | Nicotinamide nucleotide adenylyltransferase (NMNAT) | XM_015442831.3 |
| Trachypithecus francoisi | Nicotinamide nucleotide adenylyltransferase (NMNAT) | XM_033228442.1 |
| Macaca mulatta | Nicotinamide nucleotide adenylyltransferase (NMNAT) | XM_028840055.1 |
| Trachypithecus francoisi | Nicotinamide nucleotide adenylyltransferase (NMNAT) | XM_033228443.1 |
| Macaca mulatta | Nicotinamide nucleotide adenylyltransferase (NMNAT) | XM_028840044.1 |
| Macaca nemestrina | Nicotinamide nucleotide adenylyltransferase (NMNAT) | XM_011736692.2 |
| Macaca mulatta | Nicotinamide nucleotide adenylyltransferase (NMNAT) | XM_015149162.2 |
| Trachypithecus francoisi | Nicotinamide nucleotide adenylyltransferase (NMNAT) | XM_033228441.1 |
| Macaca nemestrina | Nicotinamide nucleotide adenylyltransferase (NMNAT) | XM_011736698.2 |
| Macaca thibetana thibetana | Nicotinamide nucleotide adenylyltransferase (NMNAT) | XM_050788871.1 |
| Macaca mulatta | Nicotinamide nucleotide adenylyltransferase (NMNAT) | XM_015149220.2 |
| Macaca thibetana thibetana | Nicotinamide nucleotide adenylyltransferase (NMNAT) | XM_050788868.1 |
| Macaca nemestrina | Nicotinamide nucleotide adenylyltransferase (NMNAT) | XM_011736674.2 |
| Macaca nemestrina | Nicotinamide nucleotide adenylyltransferase (NMNAT) | XM_024792074.1 |
| Macaca thibetana thibetana | Nicotinamide nucleotide adenylyltransferase (NMNAT) | XM_050788866.1 |
| Macaca nemestrina | Nicotinamide nucleotide adenylyltransferase (NMNAT) | XM_011736683.2 |
| Piliocolobus tephrosceles | Nicotinamide nucleotide adenylyltransferase (NMNAT) | XM_023215305.3 |
| Piliocolobus tephrosceles | Nicotinamide nucleotide adenylyltransferase (NMNAT) | XM_023215301.1 |
| Piliocolobus tephrosceles | Nicotinamide nucleotide adenylyltransferase (NMNAT) | XM_023215304.1 |
| Piliocolobus tephrosceles | Nicotinamide nucleotide adenylyltransferase (NMNAT) | XM_023215302.1 |
| Macaca fascicularis | Nicotinamide nucleotide adenylyltransferase (NMNAT) | XM_015442841.3 |
| Chlorocebus sabaeus | Nicotinamide nucleotide adenylyltransferase (NMNAT) | XM_037985062.1 |
| Rhinopithecus bieti | Nicotinamide nucleotide adenylyltransferase (NMNAT) | XM_017888033.1 |
| Chlorocebus sabaeus | Nicotinamide nucleotide adenylyltransferase (NMNAT) | XM_037985063.1 |
| Rhinopithecus roxellana | Nicotinamide nucleotide adenylyltransferase (NMNAT) | XM_010364176.2 |
| Chlorocebus sabaeus | Nicotinamide nucleotide adenylyltransferase (NMNAT) | XM_037985065.1 |
| Rhinopithecus bieti | Nicotinamide nucleotide adenylyltransferase (NMNAT) | XM_017888035.1 |
| Chlorocebus sabaeus | Nicotinamide nucleotide adenylyltransferase (NMNAT) | XM_037985061.1 |
| Chlorocebus sabaeus | Nicotinamide nucleotide adenylyltransferase (NMNAT) | XM_037985060.1 |
| Rhinopithecus bieti | Nicotinamide nucleotide adenylyltransferase (NMNAT) | XM_017888034.1 |
| Rhinopithecus roxellana | Nicotinamide nucleotide adenylyltransferase (NMNAT) | XM_030912911.1 |
| Chlorocebus sabaeus | Nicotinamide nucleotide adenylyltransferase (NMNAT) | XM_037985064.1 |
| Macaca mulatta | Nicotinamide nucleotide adenylyltransferase (NMNAT) | XM_028840058.1 |

In one aspect, the DNA constructs disclosed herein incorporate a gene that encodes nicotinamide riboside kinase 1 (NRK1). In a further aspect, NRK1 is expressed in various tissue including the thyroid gland, the skin, and numerous others. NRK1 is involved in the biological pathway producing NAD+ from nicotinic acid and nicotinamide (also collectively known as niacin); NRK1 and related enzymes phosphorylate nicotinamide riboside to produce NAD+.

In one aspect, the gene that encodes NRK1 is isolated from a mammal. In a further aspect, the gene that encodes NRK1 has SEQ ID NO. 3 or at least 70% homology thereto, at least 75% homology thereto, at least 80% homology thereto, at least 85% homology thereto, at least 90% homology thereto, at least 95% homology thereto, or at least 99% homology thereto.

Other sequences encoding NRK1 or related or homologous genes can be identified in a database such as, for example, GenBank. In one aspect, the gene that encodes NRK1 is isolated from a human or human cDNA and can be identified by the GI number CU691427.1 in the GenBank database. In another aspect, sequences useful herein include those with GI numbers listed in Table 3:

TABLE 3
NRK1

| Source Organism | Sequence Description | GI Number |
|---|---|---|
| synthetic construct | Synthetic construct | CU691427.1 |
| Homo sapiens | Nicotinamide riboside kinase 1 (NMRK1) | NM_001127603.2 |
| Homo sapiens | Synthetic construct | BC036804.1 |
| Gorilla gorilla gorilla | Nicotinamide riboside kinase 1 (NMRK1) | XM_004048140.4 |
| Homo sapiens | Nicotinamide riboside kinase 1 (NMRK1) | XM_054363198.1 |
| Gorilla gorilla gorilla | Nicotinamide riboside kinase 1 (NMRK1) | XM_055351170.2 |
| Gorilla gorilla gorilla | Nicotinamide riboside kinase 1 (NMRK1) | XM_063696460.1 |
| Human ORFeome Gateway | Nicotinamide riboside kinase 1 (NMRK1) | LT735054.1 |
| entry vector | | |
| Gorilla gorilla gorilla | Nicotinamide riboside kinase 1 (NMRK1) | XM_063696459.1 |
| Homo sapiens | Nicotinamide riboside kinase 1 (NMRK1) | XM_017014874.2 |
| Gorilla gorilla gorilla | Nicotinamide riboside kinase 1 (NMRK1) | XM_063696461.1 |
| synthetic construct | Synthetic construct | CU691426.1 |
| synthetic construct | Synthetic construct | KJ894109.1 |
| Pongo pygmaeus | Nicotinamide riboside kinase 1 (NMRK1) | XM_054500109.2 |
| Pan paniscus | Nicotinamide riboside kinase 1 (NMRK1) | XM_003822187.5 |
| Pongo abelii | Nicotinamide riboside kinase 1 (NMRK1) | XM_054519370.2 |
| Pan paniscus | Nicotinamide riboside kinase 1 (NMRK1) | XM_055091904.2 |
| Pan troglodytes | Nicotinamide riboside kinase 1 (NMRK1) | XM_054658135.2 |
| Pan troglodytes | Nicotinamide riboside kinase 1 (NMRK1) | XM_054658136.2 |
| Pongo abelii | Nicotinamide riboside kinase 1 (NMRK1) | XM_063713849.1 |
| Pongo abelii | Nicotinamide riboside kinase 1 (NMRK1) | XM_054519369.2 |
| Pan troglodytes | Nicotinamide riboside kinase 1 (NMRK1) | XM_001145265.6 |
| Homo sapiens | Nicotinamide riboside kinase 1 (NMRK1) | XM_054363197.1 |
| Homo sapiens | Nicotinamide riboside kinase 1 (NMRK1) | XM_047423550.1 |
| Mandrillus leucophaeus | Nicotinamide riboside kinase 1 (NMRK1) | XM_011968242.1 |
| Trachypithecus francoisi | Nicotinamide riboside kinase 1 (NMRK1) | XM_033237084.1 |
| Pongo abelii | Nicotinamide riboside kinase 1 (NMRK1) | XM_054519366.2 |
| Rhinopithecus roxellana | Nicotinamide riboside kinase 1 (NMRK1) | XM_010388727.2 |
| Chlorocebus sabaeus | Nicotinamide riboside kinase 1 (NMRK1) | XM_007969531.2 |
| Pongo abelii | Synthetic construct | CR860368.1 |
| Macaca thibetana thibetana | Nicotinamide riboside kinase 1 (NMRK1) | XM_050761977.1 |
| Macaca fascicularis | Nicotinamide riboside kinase 1 (NMRK1) | XM_005581948.4 |
| Cercocebus atys | Nicotinamide riboside kinase 1 (NMRK1) | XM_012056741.1 |
| Theropithecus gelada | Nicotinamide riboside kinase 1 (NMRK1) | XM_025360030.1 |
| Macaca mulatta | Nicotinamide riboside kinase 1 (NMRK1) | NM_001261283.1 |
| Nomascus leucogenys | Nicotinamide riboside kinase 1 (NMRK1) | XM_003267426.4 |
| Hylobates moloch | Nicotinamide riboside kinase 1 (NMRK1) | XM_032164316.2 |
| Piliocolobus tephrosceles | Nicotinamide riboside kinase 1 (NMRK1) | XM_023213305.3 |
| Colobus angolensis palliatus | Nicotinamide riboside kinase 1 (NMRK1) | XM_011947015.1 |
| Symphalangus syndactylus | Nicotinamide riboside kinase 1 (NMRK1) | XM_055272238.2 |
| Chlorocebus sabaeus | Nicotinamide riboside kinase 1 (NMRK1) | XM_007969529.2 |
| Papio anubis | Nicotinamide riboside kinase 1 (NMRK1) | XM_009188953.3 |
| Trachypithecus francoisi | Nicotinamide riboside kinase 1 (NMRK1) | XM_033237083.1 |
| Aotus nancymaae | Nicotinamide riboside kinase 1 (NMRK1) | XM_021669247.2 |
| Macaca nemestrina | Nicotinamide riboside kinase 1 (NMRK1) | XM_011770947.2 |
| Piliocolobus tephrosceles | Nicotinamide riboside kinase 1 (NMRK1) | XM_023213303.3 |
| Rhinolophus sinicus | Nicotinamide riboside kinase 1 (NMRK1) | XM_019736672.1 |
| Rhinolophus ferrumequinum | Nicotinamide riboside kinase 1 (NMRK1) | XM_033123683.1 |
| Hipposideros armiger | Nicotinamide riboside kinase 1 (NMRK1) | XM_019650680.1 |
| Artibeus jamaicensis | Nicotinamide riboside kinase 1 (NMRK1) | XM_037140331.2 |
| Dasypus novemcinctus | Nicotinamide riboside kinase 1 (NMRK1) | XM_058301659.1 |
| Talpa occidentalis | Nicotinamide riboside kinase 1 (NMRK1) | XM_037493565.2 |
| Callorhinus ursinus | Nicotinamide riboside kinase 1 (NMRK1) | XM_025891491.1 |
| Pteronotus mesoamericanus | Nicotinamide riboside kinase 1 (NMRK1) | XM_054583014.1 |
| Odobenus rosmarus divergens | Nicotinamide riboside kinase 1 (NMRK1) | XM_004396728.1 |
| Eubalaena glacialis | Nicotinamide riboside kinase 1 (NMRK1) | XM_061201180.1 |
| Balaenoptera musculus | Nicotinamide riboside kinase 1 (NMRK1) | XM_036854544.1 |
| Propithecus coquereli | Nicotinamide riboside kinase 1 (NMRK1) | XM_012644419.1 |
| Artibeus jamaicensis | Nicotinamide riboside kinase 1 (NMRK1) | XM_053656156.1 |
| Balaenoptera ricei | Nicotinamide riboside kinase 1 (NMRK1) | XM_059924936.1 |
| Eschrichtius robustus | Nicotinamide riboside kinase 1 (NMRK1) | XM_068553463.1 |
| Phyllostomus hastatus | Nicotinamide riboside kinase 1 (NMRK1) | XM_045840161.1 |
| Globicephala melas | Nicotinamide riboside kinase 1 (NMRK1) | XM_030876991.2 |
| Lipotes vexillifer | Nicotinamide riboside kinase 1 (NMRK1) | XM_007450990.1 |
| Pseudorca crassidens | Nicotinamide riboside kinase 1 (NMRK1) | XM_067744147.1 |
| Phocoena phocoena | Nicotinamide riboside kinase 1 (NMRK1) | XM_065879525.1 |
| Delphinus delphis | Nicotinamide riboside kinase 1 (NMRK1) | XM_060014616.1 |
| Mustela lutreola | Nicotinamide riboside kinase 1 (NMRK1) | XM_059141170.1 |
| Mustela erminea | Nicotinamide riboside kinase 1 (NMRK1) | XM_032306845.1 |
| Orcinus orca | Nicotinamide riboside kinase 1 (NMRK1) | XM_004276379.3 |
| Tursiops truncatus | Nicotinamide riboside kinase 1 (NMRK1) | XM_019934655.2 |
| Phacochoerus africanus | Nicotinamide riboside kinase 1 (NMRK1) | XM_047767786.1 |
| Phyllostomus hastatus | Nicotinamide riboside kinase 1 (NMRK1) | XM_045840159.1 |
| Lagenorhynchus obliquidens | Nicotinamide riboside kinase 1 (NMRK1) | XM_027105673.1 |
| Mustela lutreola | Nicotinamide riboside kinase 1 (NMRK1) | XM_059141161.1 |
| Balaenoptera acutorostrata | Nicotinamide riboside kinase 1 (NMRK1) | XM_007182282.3 |
| Mustela putorius furo | Nicotinamide riboside kinase 1 (NMRK1) | XM_004772126.3 |
| Mustela erminea | Nicotinamide riboside kinase 1 (NMRK1) | XM_032306838.1 |
| Lontra canadensis | Nicotinamide riboside kinase 1 (NMRK1) | XM_032866660.1 |
| Mustela putorius furo | Nicotinamide riboside kinase 1 (NMRK1) | XM_013045689.2 |
| Myotis myotis | Nicotinamide riboside kinase 1 (NMRK1) | XM_036330805.1 |
| Lontra canadensis | Nicotinamide riboside kinase 1 (NMRK1) | XM_032866654.1 |
| Myotis daubentonii | Nicotinamide riboside kinase 1 (NMRK1) | XM_059656930.1 |
| Chinchilla lanigera | Nicotinamide riboside kinase 1 (NMRK1) | XM_005408320.2 |
| Eptesicus fuscus | Nicotinamide riboside kinase 1 (NMRK1) | XM_054726716.1 |
| Grammomys surdaster | Nicotinamide riboside kinase 1 (NMRK1) | XM_028770108.1 |
| Myotis lucifugus | Nicotinamide riboside kinase 1 (NMRK1) | XM_014454119.2 |
| Chinchilla lanigera | Nicotinamide riboside kinase 1 (NMRK1) | XM_013506541.1 |
| Myotis brandtii | Nicotinamide riboside kinase 1 (NMRK1) | XM_014541271.1 |
| Myotis daubentonii | Nicotinamide riboside kinase 1 (NMRK1) | XM_059656926.1 |
| Rhinolophus ferrumequinum | Nicotinamide riboside kinase 1 (NMRK1) | XM_033123682.1 |
| Myotis lucifugus | Nicotinamide riboside kinase 1 (NMRK1) | XM_023752377.1 |
| Pipistrellus kuhlii | Nicotinamide riboside kinase 1 (NMRK1) | XR_006741285.1 |
| Jaculus jaculus | Nicotinamide riboside kinase 1 (NMRK1) | XM_045142291.1 |
| Hipposideros armiger | Nicotinamide riboside kinase 1 (NMRK1) | XR_002124888.1 |
| Hylobates moloch | Nicotinamide riboside kinase 1 (NMRK1) | XM_058444948.1 |
| Pongo abelii | Nicotinamide riboside kinase 1 (NMRK1) | XM_054519364.2 |
| Pongo abelii | Nicotinamide riboside kinase 1 (NMRK1) | XM_054519362.2 |
| Pongo abelii | Nicotinamide riboside kinase 1 (NMRK1) | XM_063713847.1 |
| Pongo pygmaeus | Nicotinamide riboside kinase 1 (NMRK1) | XM_054500107.2 |

In one aspect, the DNA constructs disclosed herein incorporate a gene that encodes nicotinamide riboside kinase 2 (NRK2) In a further aspect, NRK2 is involved in the biological pathway producing NAD. NRK2 is found in both the nucleoplasm and the plasma membrane and is involved in phosphorylating ribosylnicotinamide and ribosylnicotinate.

In one aspect, the gene that encodes NRK2 is isolated from a mammal. In a further aspect, the gene that encodes NRK2 has SEQ ID NO. 4 or at least 70% homology thereto, at least 75% homology thereto, at least 80% homology thereto, at least 85% homology thereto, at least 90% homology thereto, at least 95% homology thereto, or at least 99% homology thereto.

Other sequences encoding NRK2 or related or homologous genes can be identified in a database such as, for example, GenBank. In one aspect, the gene that encodes NRK2 is isolated from a human and can be identified by the GI number NM_001289117.2 in the GenBank database. In another aspect, sequences useful herein include those with GI numbers listed in Table 4:

TABLE 4
NRK2

| Source Organism | Sequence Description | GI Number |
|---|---|---|
| Homo sapiens | Nicotinamide riboside kinase 2 (NMRK2) | NM_001289117.2 |
| Homo sapiens | Integrin beta 1 binding protein 3 | BC143329.1 |
| Nomascus leucogenys | Nicotinamide riboside kinase 2 (NMRK2) | XM_012503433.2 |
| Pan troglodytes | Nicotinamide riboside kinase 2 (NMRK2) | XM_063801006.1 |
| Saimiri boliviensis boliviensis | Nicotinamide riboside kinase 2 (NMRK2) | XM_010349461.2 |
| Pongo abelii | Nicotinamide riboside kinase 2 (NMRK2) | XM_054539355.2 |
| Theropithecus gelada | Nicotinamide riboside kinase 2 (NMRK2) | XM_025368113.1 |
| Homo sapiens | Nicotinamide riboside kinase 2 (NMRK2) | AY611481.1 |
| Homo sapiens | Nicotinamide riboside kinase 2 (NMRK2) | NM_170678.3 |
| Homo sapiens | Genomic DNA | AL365377.1 |
| Homo sapiens | Integrin beta 1 binding protein 3 | BC093637.1 |
| Homo sapiens | Genomic DNA | AK022514.1 |
| Homo sapiens | Genomic DNA | AK001663.1 |
| Gorilla gorilla gorilla | Nicotinamide riboside kinase 2 (NMRK2) | XM_055371293.2 |
| Homo sapiens | Nicotinamide riboside kinase 2 (NMRK2) | XM_054320572.1 |
| Piliocolobus tephrosceles | Nicotinamide riboside kinase 2 (NMRK2) | XM_023185694.1 |
| Sapajus apella | Nicotinamide riboside kinase 2 (NMRK2) | XM_032252251.1 |
| Hylobates moloch | Nicotinamide riboside kinase 2 (NMRK2) | XM_032177910.2 |
| Nomascus leucogenys | Nicotinamide riboside kinase 2 (NMRK2) | XM_003277024.2 |
| Macaca fascicularis | Nicotinamide riboside kinase 2 (NMRK2) | XM_045379864.2 |
| Rhinopithecus bieti | Nicotinamide riboside kinase 2 (NMRK2) | XM_017847261.1 |
| Pan troglodytes | Nicotinamide riboside kinase 2 (NMRK2) | XM_024351329.3 |
| Macaca thibetana thibetana | Nicotinamide riboside kinase 2 (NMRK2) | XM_050770737.1 |
| Saimiri boliviensis boliviensis | Nicotinamide riboside kinase 2 (NMRK2) | XM_003938805.3 |
| Rhinopithecus roxellana | Nicotinamide riboside kinase 2 (NMRK2) | XM_010367268.1 |
| Symphalangus syndactylus | Nicotinamide riboside kinase 2 (NMRK2) | XM_063620837.1 |
| Pongo pygmaeus | Nicotinamide riboside kinase 2 (NMRK2) | XM_054466051.2 |
| Pongo abelii | Nicotinamide riboside kinase 2 (NMRK2) | XM_024237377.3 |
| Callithrix jacchus | Nicotinamide riboside kinase 2 (NMRK2) | XM_035286245.2 |
| Macaca mulatta | Nicotinamide riboside kinase 2 (NMRK2) | XM_028839115.1 |
| Pan paniscus | Nicotinamide riboside kinase 2 (NMRK2) | XM_034945597.3 |
| Papio anubis | Nicotinamide riboside kinase 2 (NMRK2) | XM_009193163.3 |
| Theropithecus gelada | Nicotinamide riboside kinase 2 (NMRK2) | XM_025368114.1 |
| Trachypithecus francoisi | Nicotinamide riboside kinase 2 (NMRK2) | XM_033198023.1 |
| Aotus nancymaae | Nicotinamide riboside kinase 2 (NMRK2) | XM_064382081.1 |
| Rhinopithecus roxellana | Nicotinamide riboside kinase 2 (NMRK2) | XM_030936367.1 |
| Pan paniscus | Nicotinamide riboside kinase 2 (NMRK2) | XM_034945598.3 |
| Trachypithecus francoisi | Nicotinamide riboside kinase 2 (NMRK2) | XM_033198024.1 |
| Piliocolobus tephrosceles | Nicotinamide riboside kinase 2 (NMRK2) | XM_023185695.1 |
| Chlorocebus sabaeus | Nicotinamide riboside kinase 2 (NMRK2) | XM_007994875.2 |
| Cercocebus atys | Nicotinamide riboside kinase 2 (NMRK2) | XM_012073080.1 |
| Macaca fascicularis | Nicotinamide riboside kinase 2 (NMRK2) | XM_045379863.2 |
| Sapajus apella | Nicotinamide riboside kinase 2 (NMRK2) | XM_032252252.1 |
| Cebus imitator | Nicotinamide riboside kinase 2 (NMRK2) | XM_017507393.2 |
| Capricornis sumatraensis | Nicotinamide riboside kinase 2 (NMRK2) | XM_068981148.1 |
| Sus scrofa | Nicotinamide riboside kinase 2 (NMRK2) | XM_003354004.4 |
| Macaca fascicularis | Nicotinamide riboside kinase 2 (NMRK2) | XM_045379865.2 |
| Macaca mulatta | Nicotinamide riboside kinase 2 (NMRK2) | XM_028839114.1 |
| synthetic construct | Genomic DNA | KJ905455.1 |
| Bos mutus | Nicotinamide riboside kinase 2 (NMRK2) | XM_005895954.3 |
| Bos taurus | Nicotinamide riboside kinase 2 (NMRK2) | NM_001079643.1 |
| Bos indicus × Bos taurus | Nicotinamide riboside kinase 2 (NMRK2) | XM_027547247.1 |
| Ovis aries | Nicotinamide riboside kinase 2 (NMRK2) | XM_004008606.4 |
| Ovis aries | Nicotinamide riboside kinase 2 (NMRK2) | XM_012178078.4 |
| Eubalaena glacialis | Nicotinamide riboside kinase 2 (NMRK2) | XM_061188894.1 |
| Mesoplodon densirostris | Nicotinamide riboside kinase 2 (NMRK2) | XM_060092377.1 |
| Moschus berezovskii | Nicotinamide riboside kinase 2 (NMRK2) | XM_055431431.1 |
| Oryx dammah | Nicotinamide riboside kinase 2 (NMRK2) | XM_040241959.1 |
| Bos javanicus | Nicotinamide riboside kinase 2 (NMRK2) | XM_061422649.1 |
| Globicephala melas | Nicotinamide riboside kinase 2 (NMRK2) | XM_030879270.3 |
| Capra hircus | Nicotinamide riboside kinase 2 (NMRK2) | XM_018050848.1 |
| Budorcas taxicolor | Nicotinamide riboside kinase 2 (NMRK2) | XM_052643857.1 |
| Capricornis sumatraensis | Nicotinamide riboside kinase 2 (NMRK2) | XM_068981149.1 |
| Ovis canadensis | Nicotinamide riboside kinase 2 (NMRK2) | XM_069589447.1 |
| Capra hircus | Nicotinamide riboside kinase 2 (NMRK2) | XM_018050849.1 |
| Budorcas taxicolor | Nicotinamide riboside kinase 2 (NMRK2) | XM_052643859.1 |
| Oryx dammah | Nicotinamide riboside kinase 2 (NMRK2) | XM_040241960.1 |
| Bison bison bison | Nicotinamide riboside kinase 2 (NMRK2) | XM_010828126.1 |
| Ovis canadensis | Nicotinamide riboside kinase 2 (NMRK2) | XM_069589448.1 |
| Hippopotamus amphibius kiboko | Nicotinamide riboside kinase 2 (NMRK2) | XM_057708249.1 |
| Phocoena phocoena | Nicotinamide riboside kinase 2 (NMRK2) | XM_065874495.1 |
| Orcinus orca | Nicotinamide riboside kinase 2 (NMRK2) | XM_004277204.3 |
| Lagenorhynchus albirostris | Nicotinamide riboside kinase 2 (NMRK2) | XM_060145601.1 |
| Bos taurus | Nicotinamide riboside kinase 2 (NMRK2) | XM_024994678.2 |
| Pseudorca crassidens | Nicotinamide riboside kinase 2 (NMRK2) | XM_067734226.1 |
| Phacochoerus africanus | Nicotinamide riboside kinase 2 (NMRK2) | XM_047777509.1 |
| Bubalus kerabau | Nicotinamide riboside kinase 2 (NMRK2) | XM_055547062.1 |
| Bubalus bubalis | Nicotinamide riboside kinase 2 (NMRK2) | XM_006042536.3 |
| Propithecus coquereli | Nicotinamide riboside kinase 2 (NMRK2) | XM_012646197.1 |
| Sturnira hondurensis | Nicotinamide riboside kinase 2 (NMRK2) | XM_037042739.1 |
| Myotis davidii | Nicotinamide riboside kinase 2 (NMRK2) | XM_015567208.1 |
| Phocoena sinus | Nicotinamide riboside kinase 2 (NMRK2) | XM_032626219.1 |
| Neophocaena asiaeorientalis | Nicotinamide riboside kinase 2 (NMRK2) | XM_024745943.1 |
| asiaeorientalis | | |
| Lagenorhynchus obliquidens | Nicotinamide riboside kinase 2 (NMRK2) | XM_027085507.1 |
| Eulemur rufifrons | Nicotinamide riboside kinase 2 (NMRK2) | XM_069497458.1 |
| Artibeus jamaicensis | Nicotinamide riboside kinase 2 (NMRK2) | XM_037165319.2 |
| Bison bison bison | Nicotinamide riboside kinase 2 (NMRK2) | XM_010828127.1 |
| Globicephala melas | Nicotinamide riboside kinase 2 (NMRK2) | XM_030879271.2 |
| Delphinus delphis | Nicotinamide riboside kinase 2 (NMRK2) | XM_060006537.1 |
| Tursiops truncatus | Nicotinamide riboside kinase 2 (NMRK2) | XM_033855181.1 |
| Kogia breviceps | Nicotinamide riboside kinase 2 (NMRK2) | XM_059060100.2 |
| Odocoileus virginianus | Nicotinamide riboside kinase 2 (NMRK2) | XM_020895327.2 |
| Vicugna pacos | Nicotinamide riboside kinase 2 (NMRK2) | XM_031689694.1 |
| Propithecus coquereli | Nicotinamide riboside kinase 2 (NMRK2) | XM_012646196.1 |
| Pseudorca crassidens | Nicotinamide riboside kinase 2 (NMRK2) | XM_067734229.1 |
| Lagenorhynchus albirostris | Nicotinamide riboside kinase 2 (NMRK2) | XM_060145603.1 |
| Heterocephalus glaber | Nicotinamide riboside kinase 2 (NMRK2) | XM_013065944.2 |
| Orcinus orca | Nicotinamide riboside kinase 2 (NMRK2) | XM_033400748.2 |
| Orcinus orca | Nicotinamide riboside kinase 2 (NMRK2) | XM_049707196.1 |
| Pseudorca crassidens | Nicotinamide riboside kinase 2 (NMRK2) | XM_067734228.1 |

In one aspect, the DNA constructs disclosed herein incorporate a gene that encodes quinolinate phosphoribosyltransferase (QPRT). In a further aspect, QPRT is involved in the catabolism of quinolinate. Quinolinate is an intermediate in the pathway that synthesizes nicotinamide adenine dinucleotide from tryptophan.

In one aspect, the gene that encodes QPRT is isolated from a mammal. In a further aspect, the gene that encodes QPRT has SEQ ID NO. 5 or at least 70% homology thereto, at least 75% homology thereto, at least 80% homology thereto, at least 85% homology thereto, at least 90% homology thereto, at least 95% homology thereto, or at least 99% homology thereto.

Other sequences encoding QPRT or related or homologous genes can be identified in a database such as, for example, GenBank. In one aspect, the gene that encodes QPRT is isolated from a human and can be identified by the GI number NM_014298.6 in the GenBank database. In another aspect, sequences useful herein include those with GI numbers listed in Table 5:

TABLE 5
QPRT

| Source Organism | Sequence Description | GI Number |
|---|---|---|
| Homo sapiens | Quinolinate phosphoribosyltransferase (QPRT) | NM_014298.6 |
| synthetic construct | Quinolinate phosphoribosyltransferase (QPRT) | KJ906092.1 |
| Homo sapiens | Quinolinate phosphoribosyltransferase (QPRT) | KJ534923.1 |
| Homo sapiens | Quinolinate phosphoribosyltransferase (QPRT) | BC010033.2 |
| Homo sapiens | Quinolinate phosphoribosyltransferase (QPRT) | BC018910.2 |
| synthetic construct | Quinolinate phosphoribosyltransferase (QPRT) | BT007867.1 |
| Homo sapiens | Quinolinate phosphoribosyltransferase (QPRT) | XM_054379977.1 |
| Homo sapiens | Quinolinate phosphoribosyltransferase (QPRT) | BT007231.1 |
| Human ORFeome | Quinolinate phosphoribosyltransferase (QPRT) | LT737472.1 |
| Gateway entry vector | | |
| Homo sapiens | Quinolinate phosphoribosyltransferase (QPRT) | BC005060.1 |
| synthetic construct | Quinolinate phosphoribosyltransferase (QPRT) | KJ898495.1 |
| Homo sapiens | Quinolinate phosphoribosyltransferase (QPRT) | XM_005255223.4 |
| Pan paniscus | Quinolinate phosphoribosyltransferase (QPRT) | XM_008961601.5 |
| Colobus angolensis palliatus | Quinolinate phosphoribosyltransferase (QPRT) | XM_011933566.1 |
| Pan troglodytes | Quinolinate phosphoribosyltransferase (QPRT) | XM_063796968.1 |
| Pan troglodytes | Quinolinate phosphoribosyltransferase (QPRT) | XM_016928780.4 |
| Homo sapiens | Quinolinate phosphoribosyltransferase (QPRT) | XM_054379976.1 |
| Trachypithecus francoisi | Quinolinate phosphoribosyltransferase (QPRT) | XM_033218100.1 |
| Papio anubis | Quinolinate phosphoribosyltransferase (QPRT) | XM_009198852.3 |
| Mandrillus leucophaeus | Quinolinate phosphoribosyltransferase (QPRT) | XM_011982452.1 |
| Rhinopithecus roxellana | Quinolinate phosphoribosyltransferase (QPRT) | XM_010387820.2 |
| Rhinopithecus bieti | Quinolinate phosphoribosyltransferase (QPRT) | XM_017881406.1 |
| Homo sapiens | Quinolinate phosphoribosyltransferase (QPRT) | D78177.1 |
| Macaca fascicularis | Quinolinate phosphoribosyltransferase (QPRT) | XM_045382911.2 |
| Piliocolobus tephrosceles | Quinolinate phosphoribosyltransferase (QPRT) | XM_023191265.2 |
| Cercocebus atys | Quinolinate phosphoribosyltransferase (QPRT) | XM_012089051.1 |
| Nomascus leucogenys | Quinolinate phosphoribosyltransferase (QPRT) | XM_030798481.1 |
| Macaca mulatta | Quinolinate phosphoribosyltransferase (QPRT) | XM_015125935.2 |
| Macaca nemestrina | Quinolinate phosphoribosyltransferase (QPRT) | XM_011743372.2 |
| Pongo pygmaeus | Quinolinate phosphoribosyltransferase (QPRT) | XM_054454872.2 |
| Papio anubis | Quinolinate phosphoribosyltransferase (QPRT) | XM_009198851.3 |
| Rhinopithecus bieti | Quinolinate phosphoribosyltransferase (QPRT) | XM_017881405.1 |
| Trachypithecus francoisi | Quinolinate phosphoribosyltransferase (QPRT) | XM_033218099.1 |
| Papio anubis | Quinolinate phosphoribosyltransferase (QPRT) | XM_021933280.2 |
| Pongo abelii | Quinolinate phosphoribosyltransferase (QPRT) | XM_024234064.3 |
| Symphalangus syndactylus | Quinolinate phosphoribosyltransferase (QPRT) | XM_055298870.2 |
| Theropithecus gelada | Quinolinate phosphoribosyltransferase (QPRT) | XM_025370596.1 |
| Piliocolobus tephrosceles | Quinolinate phosphoribosyltransferase (QPRT) | XM_023191264.2 |
| Gorilla gorilla gorilla | Quinolinate phosphoribosyltransferase (QPRT) | XM_063699609.1 |
| Macaca fascicularis | Quinolinate phosphoribosyltransferase (QPRT) | XM_015442211.3 |
| Macaca fascicularis | Quinolinate phosphoribosyltransferase (QPRT) | XM_065537305.1 |
| Aotus nancymaae | Quinolinate phosphoribosyltransferase (QPRT) | XM_012464511.3 |
| Macaca thibetana thibetana | Quinolinate phosphoribosyltransferase (QPRT) | XM_050773547.1 |
| Hylobates moloch | Quinolinate phosphoribosyltransferase (QPRT) | XM_032155823.2 |
| Chlorocebus sabaeus | Quinolinate phosphoribosyltransferase (QPRT) | XM_007989941.2 |
| Pongo pygmaeus | Quinolinate phosphoribosyltransferase (QPRT) | XM_054454869.2 |
| Macaca nemestrina | Quinolinate phosphoribosyltransferase (QPRT) | XM_024793126.1 |
| Macaca mulatta | Quinolinate phosphoribosyltransferase (QPRT) | XM_015125934.2 |
| Pongo pygmaeus | Quinolinate phosphoribosyltransferase (QPRT) | XM_054454870.2 |
| Callithrix jacchus | Quinolinate phosphoribosyltransferase (QPRT) | XM_002756062.6 |
| Macaca nemestrina | Quinolinate phosphoribosyltransferase (QPRT) | XM_011743371.2 |
| Macaca mulatta | Quinolinate phosphoribosyltransferase (QPRT) | XM_028841604.1 |
| Papio anubis | Quinolinate phosphoribosyltransferase (QPRT) | XM_031658322.1 |
| Pongo abelii | Quinolinate phosphoribosyltransferase (QPRT) | XM_024234063.3 |
| Gorilla gorilla gorilla | Quinolinate phosphoribosyltransferase (QPRT) | XM_063699610.1 |
| Macaca fascicularis | Quinolinate phosphoribosyltransferase (QPRT) | XM_065537304.1 |
| Gorilla gorilla gorilla | Quinolinate phosphoribosyltransferase (QPRT) | XM_019012678.4 |
| Chlorocebus sabaeus | Quinolinate phosphoribosyltransferase (QPRT) | XM_007989934.2 |
| Macaca nemestrina | Quinolinate phosphoribosyltransferase (QPRT) | XM_011743370.2 |
| Macaca mulatta | Quinolinate phosphoribosyltransferase (QPRT) | XM_028841603.1 |
| Sapajus apella | Quinolinate phosphoribosyltransferase (QPRT) | XM_032243182.1 |
| Callithrix jacchus | Quinolinate phosphoribosyltransferase (QPRT) | XM_054242760.1 |
| Cebus imitator | Quinolinate phosphoribosyltransferase (QPRT) | XM_017519221.1 |
| Saimiri boliviensis boliviensis | Quinolinate phosphoribosyltransferase (QPRT) | XM_010340688.2 |
| Pongo abelii | Quinolinate phosphoribosyltransferase (QPRT) | XM_054534849.2 |
| Eulemur rufifrons | Quinolinate phosphoribosyltransferase (QPRT) | XM_069485978.1 |
| Otolemur garnettii | Quinolinate phosphoribosyltransferase (QPRT) | XM_003795866.3 |
| Pongo abelii | Quinolinate phosphoribosyltransferase (QPRT) | XM_054534848.1 |
| Nycticebus coucang | Quinolinate phosphoribosyltransferase (QPRT) | XM_053555138.1 |
| Nycticebus coucang | Quinolinate phosphoribosyltransferase (QPRT) | XM_053555139.1 |
| Nycticebus coucang | Quinolinate phosphoribosyltransferase (QPRT) | XM_053555137.1 |
| Lemur catta | Quinolinate phosphoribosyltransferase (QPRT) | XM_045543362.1 |
| Eulemur rufifrons | Quinolinate phosphoribosyltransferase (QPRT) | XM_069485977.1 |
| Castor canadensis | Quinolinate phosphoribosyltransferase (QPRT) | XM_020157527.1 |
| Propithecus coquereli | Quinolinate phosphoribosyltransferase (QPRT) | XM_012660364.1 |
| Equus przewalskii | Quinolinate phosphoribosyltransferase (QPRT) | XM_008514290.1 |
| Elephas maximus indicus | Quinolinate phosphoribosyltransferase (QPRT) | XM_049904024.1 |
| Equus przewalskii | Quinolinate phosphoribosyltransferase (QPRT) | XM_008514288.1 |
| Equus przewalskii | Quinolinate phosphoribosyltransferase (QPRT) | XM_008514289.1 |
| Equus asinus | Quinolinate phosphoribosyltransferase (QPRT) | XM_014842418.3 |
| Artibeus jamaicensis | Quinolinate phosphoribosyltransferase (QPRT) | XM_037158336.2 |
| Equus quagga | Quinolinate phosphoribosyltransferase (QPRT) | XM_046668491.1 |
| Delphinapterus leucas | Quinolinate phosphoribosyltransferase (QPRT) | XM_022588309.1 |
| Balaenoptera ricei | Quinolinate phosphoribosyltransferase (QPRT) | XM_059896725.1 |
| Pteropus vampyrus | Quinolinate phosphoribosyltransferase (QPRT) | XM_011378813.1 |
| Rattus norvegicus | Quinolinate phosphoribosyltransferase (QPRT) | XM_006230246.5 |
| Rattus norvegicus | Quinolinate phosphoribosyltransferase (QPRT) | NM_001009646.1 |
| Equus caballus | Quinolinate phosphoribosyltransferase (QPRT) | XM_005598783.4 |
| Equus asinus | Quinolinate phosphoribosyltransferase (QPRT) | XM_070484591.1 |
| Equus caballus | Quinolinate phosphoribosyltransferase (QPRT) | XM_070231943.1 |
| Equus quagga | Quinolinate phosphoribosyltransferase (QPRT) | XM_046668490.1 |
| Trichechus manatus latirostris | Quinolinate phosphoribosyltransferase (QPRT) | XM_023740975.1 |
| Phyllostomus hastatus | Quinolinate phosphoribosyltransferase (QPRT) | XM_045849156.1 |
| Elephas maximus indicus | Quinolinate phosphoribosyltransferase (QPRT) | XM_049904023.1 |
| Acomys russatus | Quinolinate phosphoribosyltransferase (QPRT) | XM_051145299.1 |
| Loxodonta africana | Quinolinate phosphoribosyltransferase (QPRT) | XM_023558930.2 |
| Rattus norvegicus | Quinolinate phosphoribosyltransferase (QPRT) | FQ209866.1 |
| Chrysochloris asiatica | Quinolinate phosphoribosyltransferase (QPRT) | XM_006877908.1 |
| Microcebus murinus | Quinolinate phosphoribosyltransferase (QPRT) | XM_012764249.1 |
| Equus caballus | Quinolinate phosphoribosyltransferase (QPRT) | XM_070231942.1 |

In any of these aspects, the additional DNA constructs useful for producing NAD can have SEQ ID NO 6.

### Additional DNA Constructs

In some aspects, in addition to lysates and extracts from the DNA constructs expressing NAD described above, lysates and extracts from one or more additional DNA constructs may be added to the disclosed compositions in order to supplement the nutrition provided by the NAD. In an aspect, these additional DNA constructs may provide one or more vitamins or antioxidants such as, for example, lycopene, or may provide a flavorant or sweetener such as, for example, steviol glycosides, an organic electrolyte, or another beneficial compound.

### Further Components of the NAD-Producing DNA Constructs

In one aspect, the NAD-producing DNA construct has the following genetic components:
a) a gene that encodes NAMPT, b) a gene that encodes NMNAT, c) a gene that encodes NRK1,
d) a gene that encodes NRK2, and e) a gene that encodes QPRT.

In another aspect, said construct further includes a) a promoter, b) a terminator or stop sequence, c) a gene that confers resistance to an antibiotic (a “selective marker”), d) a reporter protein, or any combination thereof. Each of these elements is described in further detail below.

In one aspect, the construct includes from 5′ to 3′ the following genetic components in the following order: (1) a gene that encodes NAMPT, (2) a gene that encodes NMNAT, (3) a gene that encodes NRK1, (4) a gene that encodes NRK2, and (5) a gene that encodes QPRT.

In one aspect, the construct includes from 5′ to 3′ the following genetic components in the following order: a gene that encodes NAMPT having SEQ ID NO. 1 or at least 70% homology thereto, a gene that encodes NMNAT having SEQ ID NO. 2 or at least 70% homology thereto, a gene that encodes NRK1 having SEQ ID NO. 3 or at least 70% homology thereto, a gene that encodes NRK2 having SEQ ID NO. 4 or at least 70% homology thereto, and a gene that encodes QPRT having SEQ ID NO. 5 or at least 70% homology thereto.

In another aspect, the construct includes from 5′ to 3′ the following genetic components in the following order: (1) a gene that encodes NAMPT, (2) a CYC1 terminator, (3) a GAL 1 promoter, (4) a gene that encodes NMNAT, (5) a CYC1 terminator, (6) a GAL1 promoter, (7) a gene that encodes NRK1, (8) a CYC1 terminator, (9) a GAL1 promoter, (10) a gene that encodes NRK2; (11) a CYC1 terminator; (12) a GAL1 promoter; and (13) a gene that encodes QPRT.

In another aspect, the construct includes from 5′ to 3′ the following genetic components in the following order: (1) a gene that encodes NAMPT having SEQ ID NO. 1 or at least 90% homology thereto, (2) a CYC1 terminator, (3) a GAL1 promoter, (4) a gene that encodes NMNAT having SEQ ID NO. 2 or at least 90% homology thereto, (5) a CYC1 terminator, (6) a GAL1 promoter, (7) a gene that encodes NRK1 having SEQ ID NO. 3 or at least 90% homology thereto, (8) a CYC1 terminator, (9) a GAL1 promoter, (10) a gene that encodes NRK2 having SEQ ID NO. 4 or at least 90% homology thereto; (11) a CYC1 terminator; (12) a GAL1 promoter; and (13) a gene that encodes QPRT having SEQ ID NO. 5 or at least 90% homology thereto.

In still another aspect, the construct is a pYES2 plasmid having from 5′ to 3′ the following genetic components in the following order: (1) a gene that encodes NAMPT having SEQ ID NO. 1 or at least 70% homology thereto, (2) a CYC1 terminator, (3) a GAL1 promoter, (4) a gene that encodes NMNAT having SEQ ID NO. 2 or at least 70% homology thereto, (5) a CYC1 terminator, (6) a GAL1 promoter, (7) a gene that encodes NRK1 having SEQ ID NO. 3 or at least 70% homology thereto, (8) a CYC1 terminator, (9) a GAL1 promoter, (10) a gene that encodes NRK2 having SEQ ID NO. 4 or at least 70% homology thereto; (11) a CYC1 terminator; (12) a GAL1 promoter; and (13) a gene that encodes QPRT having SEQ ID NO. 5 or at least 70% homology thereto.

In another aspect, the DNA construct has SEQ ID NO. 6 or at least 70% homology thereto, at least 75% homology thereto, at least 80% homology thereto, at least 85% homology thereto, at least 90% homology thereto, at least 95% homology thereto, or at least 99% homology thereto.

### Additional Components of the DNA Constructs

In another aspect, said construct further includes a) a promoter, b) a terminator or stop sequence, c) a gene that confers resistance to an antibiotic (a “selective marker”), d) a reporter protein, or any combination thereof.

In one aspect, the construct includes a regulatory sequence. In a further aspect, the regulatory sequence is already incorporated into a vector such as, for example, a plasmid, prior to genetic manipulation of the vector. In another aspect, the regulatory sequence can be incorporated into the vector through the use of restriction enzymes or any other technique known in the art.

In one aspect, the regulatory sequence is a promoter. The term “promoter” refers to a DNA sequence capable of controlling the expression of a coding sequence. In another aspect, the coding sequence to be controlled is located 3′ to the promoter. In still another aspect, the promoter is derived from a native gene. In an alternative aspect, the promoter is composed of multiple elements derived from different genes and/or promoters. A promoter can be assembled from elements found in nature, from artificial and/or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, such as, for example, in response to the presence or absence of a particular metabolite (e.g., lactose or tryptophan), a metal ion, a molecule secreted by a pathogen, or the like. A promoter also may be regulated physically, such as, for example, in response to heat, cold, water stress, salt stress, oxygen concentration, illumination, wounding, or the like.

Promoters that are useful to drive expression of the nucleotide sequences described herein are numerous and familiar to those skilled in the art. Suitable promoters include, but are not limited to, the following: T3 promoter, T7 promoter, an iron promoter, araBAD promoter, and GAL1 promoter. In a further aspect, the promoter is a native part of the vector used herein. Variants of these promoters are also contemplated. The skilled artisan will be able to use site-directed mutagenesis and/or other mutagenesis techniques to modify the promoters to promote more efficient function. The promoter may be positioned, for example, from 10-100 nucleotides from a ribosomal binding site.

In one aspect, the promoter is a GAL1 promoter. In another aspect, the GAL1 promoter is native to the plasmid used to create the vector. In another aspect, a GAL1 promoter is positioned before the gene that encodes NAMPT, the gene that encodes NMNAT, the gene that encodes NRK1, the gene that encodes NRK2, the gene that encodes QPRT, or any combination thereof.

In one aspect, the regulatory sequence is an operon such as, for example, the LAC operon or LAC operator. As used herein, an “operon” is a segment of DNA containing a group of genes wherein the group is controlled by a single promoter. Genes included in an operon are all transcribed together. In a further aspect, the operon is a LAC operon and can be induced when lactose crosses the cell membrane of the biological device.

In another aspect, the regulatory sequence is a terminator or stop sequence. As used herein, a terminator is a sequence of DNA that marks the end of a gene or operon to be transcribed. In a further aspect, the terminator is an intrinsic terminator or a Rho-dependent transcription terminator. As used herein, an intrinsic terminator is a sequence wherein a hairpin structure can form in the nascent transcript that disrupts the mRNA/DNA/RNA polymerase complex. As used herein, a Rho-dependent transcription terminator requires a Rho factor protein complex to disrupt the mRNA/DNA/RNA polymerase complex. In one aspect, the terminator is an rrnB terminator obtained from or native to the pBAD plasmid. In an alternative aspect, the terminator is a CYC1 terminator obtained from or native to the pYES2 plasmid.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2000 nucleotides in the 5′ direction from the start codon of the DNA to be transcribed, or may be located 3′ to the DNA to be transcribed. Enhancers may be “cis-acting,” that is, located on the same molecule of DNA as the gene whose expression they affect.

In one aspect, and without wishing to be bound by theory, when the plasmid is a pYES2 plasmid, genes from the plasmid are ideally suited for expression in yeast under the control of the GAL1 promoter and CYC1 terminator. In a further aspect, since the expression of multiple genes is regulated under the same type of promoter, all genes arranged sequentially in a group having a 5′ GAL1 promoter and/or ending with an CYC1 terminator can be expressed together. In an alternative aspect, each gene can have its own individual GAL1 promoter situated 5′ to the gene and its own CYC1 terminator situated 3′ to the gene.

### Further Components of the DNA Constructs and Methods for Making Thereof

In another aspect, the vector contains one or more ribosomal binding sites. As used herein, a “ribosomal binding site” or “rbs” is a sequence of nucleotides located 5′ to the start codon of an mRNA that recruits a ribosome to initiate protein translation. In one aspect, the ribosomal binding site can be positioned before one or more or all genes in the DNA construct, or a before a subset of genes in a DNA construct.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for restriction enzymes. Further, restriction sites may be added, disabled, or removed as required, using techniques known in the art. In one aspect, the plasmid contains restriction sites for any known restriction enzyme such as, for example, HindIII, KpnI, SacI, BamHI, BstXI, EcoRI, BasBI, NotI, XhoI, XphI, XbaI, ApaI, SalI, ClaI, EcoRV, PstI, SmaI, XmaI, Spel, EagI, SacII, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA at a distance of from 20 bases to over 1000 bases away from the restriction site. A variety of restriction enzymes are commercially available and their recognition sequences, as well as instructions for use (e.g., amount of DNA needed, precise volumes of reagents, purification techniques, as well as information about salt concentration, pH, optimum temperature, incubation time, and the like) are provided by enzyme manufacturers.

In one aspect, a plasmid with a polylinker containing one or more restriction sites can be digested with one restriction enzyme and a nucleotide sequence of interest can be ligated into the plasmid using a commercially-available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, usually starting just after a promoter, the order and direction of elements inserted into a plasmid can be especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleotide fragments into the plasmid.

In one aspect, different genes can be ligated into a plasmid in one pot. In this aspect, the genes will first be digested with restriction enzymes. In certain aspects, the digestion of genes with restriction enzymes provides multiple pairs of matching 5′ and 3′ overhangs that will spontaneously assemble the genes in the desired order. In another aspect, the genes and components to be incorporated into a plasmid can be assembled into a single insert sequence prior insertion into the plasmid. In a further aspect, a DNA ligase enzyme can be used to assist in the ligation process.

In another aspect, the ligation mix may be incubated in an electromagnetic chamber. In one aspect, the incubation lasts for about 1 minute, about 2 minutes, about 5 minutes, about 10 minutes, about 15 minutes, about 20 minutes, about 30 minutes, or about 1 hour.

The DNA construct described herein can be part of a vector. In general, plasmid vectors containing replicon and control sequences that are derived from species compatible with the host cell are used in connection with the hosts. The vector ordinarily carries a replication site as well as marking sequences that are capable of performing phenotypic selection in transformed cells. Plasmid vectors are well known and commercially available. Such vectors include, but are not limited to, pWLneo, pSV2cat, pOG44, pXT1, pSG, pSVK3, pBSK, pYES, pYES2, pBSKII, pET, pUC, pUC19, pBAD, and pETDuet-1 vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copier per host cell and will contain selectable markers (such as genes for antibiotic resistance) to show the skilled artisan to select host eels that have been successfully transfected with the plasmids (for example, by growing the host cells in a medium containing the antibiotic).

In one aspect, the vector encodes a selection marker. In a further aspect, the selection marker is a gene that confers resistance to an antibiotic. In certain aspects, during fermentation of host cells transformed with the vector, the cells are contacted with the antibiotic. For example, the antibiotic may be included in the culture medium. Cells that have not been successfully transformed cannot survive in the presence of the antibiotic; only cells containing the vector, which confers antibiotic resistance, can survive. Optimally, only cells containing the vector to be expressed will be cultured, as this will result in the highest production efficiency of the desired gene products (e.g., peptides). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isonizid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

In certain aspects, the DNA construct can include a gene that encodes a reporter protein. The selection of the reporter protein can vary. For example, the reporter protein can be a yellow fluorescent protein, a red fluorescent protein, a green fluorescent protein, or a cyan fluorescent protein. In one aspect, the reporter protein is an enhanced green fluorescent protein and the gene that encodes the reporter protein has SEQ ID NO. 8 or at least 70% homology thereto. In an alternative aspect, the reporter protein is a yellow fluorescent protein and the gene that encodes the reporter protein has SEQ ID NO. 7 or at least 70% homology thereto. The amount of fluorescence that is produced can be correlated to the amount of DNA incorporated into the transfected cells. The fluorescence produced can be detected and quantified using techniques known in the art. For example, spectrofluorometers are typically used to measure fluorescence. In some aspects, the additional DNA constructs useful for producing carotenoids, steviol glycosides, and/or organic electrolytes can also include genes encoding the reporter proteins described herein.

The DNA construct described herein can be part of a vector. In one aspect, the vector is a plasmid, a phagemid, a cosmid, a yeast artificial chromosome, a bacterial artificial chromosome, a virus, a phage, or a transposon.

Exemplary methods for producing the DNA constructs described herein are provided in the Examples. Restriction enzymes and purification techniques known in the art can be used to assemble the DNA constructs. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the methods described below.

### Cells and Biological Devices

A variety of different types of cells can be used in the methods described herein. In one aspect, the cells can be wild-type cells (i.e., not genetically-modified). In one aspect, the cells are from an animal such as, for example, a mammal, bird, fish, reptile, amphibian, or invertebrate. In another aspect, the cells are from a plant such as, for example, an agricultural crop, a decorative plant, a woody plant, a medicinal plant, or a combination thereof. In another aspect, the cells are from a multicellular fungus such as, for example, a mushroom, a mycorrhizal fungus, or a commercially-important mold.

In another aspect, the cells include a biological device. A “biological device” is formed when a microbial cell is transfected with a DNA construct. The biological devices are generally composed of microbial host cells, where the host cells are transformed (i.e., genetically-modified) with a DNA construct.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce an extract or composition that can remove metals and/or other contaminants from water or petroleum, such as, for example, those disclosed herein. “Heterologous” genes and proteins are genes and proteins that have been experimentally inserted into a cell that are not normally expressed by the cell. A heterologous gene may be cloned or derived from a different cell type or species than the recipient cell or organism. Heterologous genes may be introduced into cells by transduction or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes).

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the type of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed only for a limited period of time).

“Competent cells” refers to microbial cells capable of taking up heterologous DNA. Competent cells can be purchased from a commercial source, or cells can be made competent using procedures known in the art. Exemplary procedures for producing competent cells are provided in the Examples.

The host cells as referred to herein include their progeny, which are any and all subsequent generations formed by cell division. It is understood that not all progeny may be identical due to deliberate or inadvertent mutations. A host cell may be “transfected” or “transformed,” which refers to a process by which an exogenous nucleic acid is transferred or introduced into the host cell.

A transformed cell includes the primary subject cell and its progeny. The host cells can be naturally-occurring cells or “recombinant” cells. Recombinant cells are distinguishable from naturally-occurring cells in that naturally-occurring cells do not contain heterologous DNA introduced through molecular cloning procedures. In one aspect, the host cell is a prokaryotic cell such as, for example, Escherichia coli. In other aspects, the host cell is a eukaryotic cell such as, for example, the yeast Saccharomyces cerevisiae. Host cells transformed with the DNA construct described herein are referred to as “biological devices.”

The DNA construct is first delivered into the host cell. In one aspect, the host cells are naturally competent (i.e., able to take up exogenous DNA from the surrounding environment). In another aspect, cells must be treated to induce artificial competence. This delivery may be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the membranes of the cells through which the vector containing the DNA construct enters. Another method involves exposing intact yeast cells to alkali cations such as, for example, lithium. In one aspect, this method includes exposing yeast to lithium acetate, polyethylene glycol, and single-stranded DNA such as, for example, salmon sperm DNA. Without wishing to be bound by theory, the single-stranded DNA is thought to bind to the cell wall of the yeast, thereby blocking plasmids from binding. The plasmids are then free to enter the yeast cell. Enzymatic and/or electromagnetic techniques can also be used alone, or in combination with other methods, to transform microbial cells. Exemplary procedures for transforming yeast and bacteria with specific DNA constructs are provided in the Examples. In certain aspects, two or more types of DNA can be incorporated into the host cells. Thus, different metabolites can be produced from the same host cells at enhanced rates.

### Cell Culture

A satisfactory microbiological culture contains available sources of hydrogen donors and acceptors, carbon, nitrogen, sulfur, phosphorus, inorganic salts and, in certain cases, vitamins or other growth-promoting substances. For example, the addition of peptone provides a readily-available source of nitrogen and carbon. Furthermore, the use of different types of media results in different growth rates and different stationary phase densities. A rich media results in a short doubling time and higher cell density at stationary phase. Minimal media results in slow growth and low final cell densities. Efficient agitation and aeration increase final cell densities. In some aspects, device culture and metabolite extraction are performed in the absence of natural or artificial light, in order to prevent decrease of the efficacy of NAD.

Culturing or fermenting of host cells can be accomplished by any technique known in the art. In one aspect, batch fermentation can be conducted. In batch fermentation, the composition of the culture medium is set at the beginning and the system is closed to future alterations. In some aspects, a limited form of batch fermentation may be carried out, wherein factors such as oxygen concentration and pH are manipulated, but additional carbon is not added. Continuous fermentation methods are also contemplated. In continuous fermentation, equal amounts of a defined medium are continuously added to and removed from a bioreactor. In other aspects, microbial cells are immobilized on a substrate. Fermentation may be carried out on any scale and may include methods in which literal “fermentation” is carried out as well as other culture methods that are non-fermentative. In one aspect, the host cells can be cultured in yeast malt media or a combination of yeast malt media and molasses at different concentrations. In a further aspect, the device culture can be induced during growth using raffinose, galactose, and/or glucosamine, depending on the specific device. In a further aspect, concentration of media can be adjusted to maintain a balance between cell growth/division and production of metabolites. In some aspects, too much cell growth prevents expression of the metabolite.

In one aspect, the microorganisms can be cultured for a period of from 2 days to 2 weeks, or for about 2, about 3, about 4, about 5, about 6, about 7, about 8, about 9, about 10, about 11, about 12, about 13, or about 14 days, where any value can be the lower or upper endpoint of a range (e.g., about 3 days to about 13 days, about 8 days to about 12 days, etc.). In one aspect, the microorganisms are cultured for about 10 days.

In another aspect, the microorganisms can be cultured at any temperature appropriate for the microorganisms, with the understanding that the temperature may vary according to the microorganism (for example, a thermophilic microorganism may require a higher culture temperature than a mesophile). In one aspect, the microorganisms are cultured at a temperature of from about 20 to about 37° C., or are cultured at about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C., where any value can be the lower or upper endpoint of a range, where any value can be the lower or upper endpoint of a range (e.g., about 21° C. to about 36° C., about 25° C. to about 30° C., etc.).

In certain aspects, after culturing the microorganisms for a sufficient time, the microbial cells can be lysed with one or more enzymes. For example, when the microbial cells are fungal, the fungal cells can be lysed with lyticase. In one aspect, the lyticase concentration can be about 500 μL, about 600 μL, about 700 μL, about 800 μL, about 900 μL, or about 1,000 μL per liter of culture, where any value can be the lower or upper endpoint of a range, where any value can be the lower or upper endpoint of a range (e.g., about 500 μL to about 900 μL, about 600 μL to about 800 μL, etc.).

In addition to or in place of enzymes, other components can be used to facilitate lysis of the microbial cells. In one aspect, chitosan can be used in combination with an enzyme to lyse the microbial cells. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation. In one aspect, the chitosan is from about 60% to about 100% acetylated, or about 70%, about 75%, about 80%, about 85%, about 90%, about 95%, or about 100% acetylated, where any value can be the lower or upper endpoint of a range, where any value can be the lower or upper endpoint of a range (e.g., about 60% to about 90%, about 70% to about 80%, etc.).

The molecular weight of the chitosan can vary, as well. For example, the chitosan can comprise about 3, 4, 5, 6, 7, 8, 9, 10, 11, 12, 13, 14, 15, 16, 17, 18, 19, or 20 glucosamine units and/or N-acetylglucosamine units, where any value can be the lower or upper endpoint of a range, where any value can be the lower or upper endpoint of a range (e.g., 2 to 19, 3 to 10, 5 to 7, etc.). In one aspect, chitosan can be added until a concentration of about 0.0015%, about 0.0025%, about 0.005%, about 0.0075%, about 0.01%, about 0.015%, about 0.02%, about 0.03%, about 0.04%, or about 0.05%, where any value can be an upper or lower endpoint of a range (e.g., 0.002% to 0.04%, 0.05% to 0.015%, etc.).

In another aspect, cells can first be fermented, for example, in a biofermenter, at a temperature conducive to cell growth. In one aspect, the cells are fermented at 30° C. In a further aspect, the cells are fermented for a time period sufficient to produce the metabolite(s) of interest. In one aspect, the cells are fermented for from 6 hours to 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, during fermentation, a micro-current can be applied to the cells as described above. In some aspects, the micro-current is applied for the entire culture period. In another aspect, the micro-current is applied for only a part of the culture period, or for several non-consecutive parts of the culture period. In one aspect, the micro-current is the same throughout the entire culture period. In an alternative aspect, the micro-current is varied during the culture period.

Exemplary methods for culturing cells and/or the biological devices disclosed herein are provided in the Examples.

### Extraction and Purification of Metabolites

In one aspect, the methods disclosed herein can be used to increase the production of metabolites by cells. In some aspects, the metabolites are secreted into a culture medium and collected. In other aspects, the metabolites remain in the cells, requiring the cells to be lysed prior to collection and purification of the metabolites. In one aspect, the metabolites are NAD+, NADP+, NADH, and/or NADPH.

In one aspect, prior to collection of any metabolite(s) of interest, fermentation can be stopped. In some aspects, the micro-current will be withdrawn or turned off (e.g., by turning off a power supply to a biofermenter or a similar mechanism). In another aspect, an enzyme such as, for example, lyticase can optionally be used to lyse cells following fermentation. In still another aspect, the cell culture can optionally be autoclaved for a sufficient time following cell lysis in order to ensure no living cells remain in the culture. Following lysis and autoclaving, or instead of performing these two processes, centrifugation, sonication, and filtration can be performed to facilitate collection of relevant metabolites. In an alternative aspect, culture medium including an increased concentration of the desired metabolite(s) from the biofermenter can be used without further processing.

### Use of Micro-Current to Enhance Cell Culture

As used herein, “micro-current” refers to an electric current of from about 50 mV to about 300 mV. In one aspect, when a micro-current is applied to cells (e.g., a substrate on which the cells are growing or a culture medium in which the cells are growing) via one or more electrodes, cell growth and division and/or the production of one or more desirable metabolites from the cells is increased.

“Growth” as used herein refers to an increase in size or population of cells, or both, whereas “increase” used with respect to a treated population of cells (e.g., exposed to micro-current) refers to the treated cells having larger size and/or to the presence of a higher number of cells or colony forming units (CFUs) compared to a reference population of identical but untreated cells (i.e., no exposure to the micro-current) after a predetermined period of time.

“Metabolites” are any substances produced during metabolism. In some aspects, metabolites are essential to cellular function (e.g., glycolysis intermediates) or cell signaling, or can be produced by organisms in times of stress (e.g., heat shock proteins), or are compounds produced by plants or microorganisms that have been found to have functions useful in industry and/or medicine (e.g., vitamins, antibiotics, sweeteners, or the like). Metabolites can be purified (e.g., lycopene and other carotenoids) or can be used as crude extracts (e.g., the organic electrolytes and anti-microbial compounds disclosed herein). In any of these aspects, exposure to a micro-current causes treated cells to increase production of one or more metabolites.

“Production” as used herein refers to the making of one or more metabolites by cells. In one aspect, the metabolites are secreted by the cells into a culture medium. In an alternative aspect, the metabolites remain in the cell interiors and the cells must be lysed to release the metabolites. Metabolite production can be accomplished via one of the following means: (1) for peptide and protein metabolites, transcription of DNA to RNA and translation of RNA to a protein; (2) for non-peptide metabolites, transcription and translation of one or more proteins followed by protein-based catalysis of reactions that transform one or more precursor molecules (e.g., lipids, sugars, amino acids, nucleotides and nucleotide components, and other small molecules) into the desired metabolite; and (3) other methods known in the art but not already listed. In one aspect, exposure to a micro-current as disclosed herein can increase the production of one or more metabolites by cells.

In one aspect, disclosed herein is a method for growing cells, wherein the method includes exposing the cells to a micro-current during the growth of the cells. In another aspect, exposing the cells to the micro-current increases cell population as compared to identical cells that are not exposed to the micro-current.

### Micro-Current

In one aspect, a micro-current is applied to the cells and/or biological devices disclosed herein. In one aspect, the micro-current is from about 50 to about 300 mV, or is about 50, 75, 100, 120, 125, 150, 175, 200, 220, 225, 250, 275, or about 300 mV, or a combination of any of the foregoing values, or a range encompassing any of the foregoing values. In one aspect, the micro-current is about 120 mV. In another aspect, the micro-current is about 200 mV. In an aspect, regardless of the micro-current voltage, metabolite production and cellular growth are higher than for otherwise identical cells in the absence of a micro-current. However, in one aspect, a higher micro-current (e.g. 200 mV) causes colony size to increase compared to a lower (e.g. 120 mV) micro-current. In another aspect, lower micro-current (e.g. 120 mV) may result in a larger number of colonies but with each individual colony having a smaller size than with a higher micro-current (e.g. 200 mV). In any of these aspects, then, the desired outcome (size of colonies, colony count, or both) can be tailored to a specific project. In some aspects, the micro-current is adjusted in order to maintain a balance between cell growth/division and production of metabolites. In some aspects, excess or deficiency of microcurrent can prevent cell growth or expression of the metabolite.

In one aspect, the cells are exposed to the micro-current using at least one electrode. In a further aspect, the electrode is connected to a power supply. In some aspects, the power supply can be adjusted to provide variable voltages for the micro-current. In an alternative aspect, the electrode can be connected to a battery. In any of these aspects, the at least one electrode can be made from copper, graphite, carbon nanotubes, graphene, titanium, brass, silver, platinum, palladium, iron, nickel, lead, steel, magnesium, aluminum, tin, zinc, tungsten, mixed metal oxides, a spinel-type structure, an olivine-type structure, or a combination thereof. In some aspects, the at least one electrode is made from platinum. In another aspect, if more than one electrode is used in the processes disclosed herein, the electrodes can be made from two different materials (e.g., graphene and platinum).

In another aspect, the cells and/or biological devices disclosed herein are exposed to a micro-current for from about 6 hours to about 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, the cells and/or biological devices disclosed herein are exposed to a micro-current for about 48 hours.

In another aspect, cells can first be fermented, for example, in a biofermenter, at a temperature conducive to cell growth. In one aspect, the cells are fermented at 30° C. In a further aspect, the cells are fermented for a time period sufficient to produce the metabolite(s) of interest. In one aspect, the cells are fermented for from 6 hours to 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, during fermentation, a micro-current can be applied to the cells as described above. In some aspects, the micro-current is applied for the entire culture period. In another aspect, the micro-current is applied for only a part of the culture period, or for several non-consecutive parts of the culture period. In one aspect, the micro-current is the same throughout the entire culture period. In an alternative aspect, the micro-current is varied during the culture period.

Exemplary methods for culturing cells and/or the biological devices disclosed herein are provided in the Examples.

In some aspects, the cells are suspended in a culture medium. In another aspect, the culture medium can be Dulbecco's Modified Eagle Medium (DMEM), RPMI 1640, Minimal Essential Medium (MEM), Eagle's Minimal Essential Medium (EMEM), Iscove's Modified Dulbecco's Medium (IMDM), DMEM/F12 Medium, Murashige and Skoog (MS) medium, White's medium, Agrobacterium minimal medium, Banana AGS basal medium, Blaydes basal medium, Bold's basal medium, Chu (N6) medium, De Greef and Jacobs Medium, DKW basal medium, Economou and Read basal medium, Gamborg (B5) medium, Gresshoff and Doy medium, Heller medium, Hoagland complete medium, Jensen's medium, Kao and Michayluk medium, Litvay medium, NB basal medium, Nitsch medium. NLN medium, Quoirin and Lepoivre medium, Schenk and Hildebrandt medium, TAP medium, TM4G medium, Vacin and Went medium, wheat callus induction medium, Luria Bertani (LB) broth, terrific broth, tryptic soy broth, minimal salts (M9) medium, SOB medium, SOC medium, yeast malt medium, YPD broth, YNB broth, synthetic complete (SC) medium, YPG medium, Hartwell's complete (HC) medium, or a combination thereof. In one aspect, the culture medium is Luria Bertani (LB) broth or yeast malt medium.

In another aspect, the culture medium can contain supplemental compounds such as, for example, vitamins, nucleosides, nucleotides, amino acids, a carbohydrate, an antibiotic, or a combination thereof.

In one aspect, the culture medium can be a liquid. In another aspect, the methods disclosed herein can be performed in a biofermenter. In an alternative aspect, the cells can be distributed on a substrate. In one aspect, the substrate can be agar, a culture dish, contaminated soil, a wastewater treatment device, mineral ore, a plant organ, a tissue scaffold, or a fermentable material. When the substrate is a plant organ, in some aspects, the plant organ can be a root, leaf, stem, rhizome, tuber, flower, seed, fruit, vegetable, callus, or a combination thereof. When the substrate is a fermentable material, in some aspects, the substrate can be milk, a grain, cabbage, soybeans, fish, or a biomass feedstock. When the substrate is a biomass feedstock, in some aspects, the substrate can be forestry residue, logging residue, sawmill residue, animal manure, a recycled material, a carbohydrate waste, corn cob, corn stover, wheat straw, nut hulls, soy hulls, switchgrass, gammagrass, paper, or a combination thereof.

In one aspect, when the cells to be exposed to a micro-current are in a liquid medium (e.g., in a biofermenter), the at least one electrode can be immersed in the liquid medium. In an alternative aspect, the biofermenter can have one or more panels, elements, or contact points built into the biofermenter walls, wherein the panels, elements, or contact points are made from electrode materials as disclosed herein, and wherein the panels, elements, or contact points come into contact with the liquid culture medium.

In still another aspect, when the cells to be exposed to a micro-current are in contact with a substrate, the manner of contact with the at least one electrode with the substrate will depend on the characteristics of the substrate. In a further aspect, if the substrate is a solid material such as, for example, a component of a wastewater treatment device or a tissue scaffold, the substrate can include a conductive material such as, for example, copper, graphite, carbon nanotubes, graphene, titanium, brass, silver, platinum, palladium, iron, nickel, lead, steel, magnesium, aluminum, tin, zinc, tungsten, mixed metal oxides, a spinel-type structure, an olivine-type structure, or a combination thereof. In a further aspect, the conductive material can be present as a wire or network of wires, as a coating, as the substrate itself, or any other form useful for and conducive to the passage of micro-current through the cells. In an alternative aspect, an electrolyte solution or gel can be applied to the substrate in order to facilitate the passage of the micro-current through the cells. In any of these aspects, the cells can be applied to the substrate using any means known in the art and the at least one electrode can be placed into direct contact with the substrate, conductive material, or electrolyte in order to apply the micro-current to the cells.

In one aspect, when the substrate is a material intended for further processing or industrial use (e.g., a fermentable material, contaminated soil, a mineral ore, or the like), the substrate can optionally be milled, crushed, and/or ground to reduce the particle size (as in a mineral ore) to increase the surface area of the substrate. In a further aspect, the substrate can optionally be contacted with a liquid suitable for sustaining cellular life and growth along with the cells intended to perform the processing (e.g., cells to ferment the material, to decontaminate the soil, to extract the mineral from the ore, or another function). Further in this aspect, the liquid can be capable of conducting electricity. In still another aspect, following processing as described herein, the substrate can be contacted with the at least one electrode by a means such as, for example, immersing or burying the electrode in the substrate.

In any of the above aspects, the cells can be genetically modified. In an alternative aspect, the cells are not genetically-modified. In some aspects, the cells can include both genetically-modified and non-genetically-modified cells.

In one aspect, application of a micro-current as disclosed herein increases cell growth. In a further aspect, increased cell growth can be evaluated by culturing identical cells in two groups, wherein one group is exposed to a micro-current and the other group is not exposed to a micro-current. After a given culture period, the group exposed to the micro-current according to the methods disclosed herein will contain a greater number of colony forming units (CFUs) compared to identical cells that were not exposed to a micro-current. In another aspect, the group exposed to the micro-current according to the method disclosed herein will, if capable of producing a metabolite, have produced a greater amount of the metabolite compared to identical cells that were not exposed to a micro-current. In still another aspect, the group exposed to the micro-current according to the method disclosed herein will contain larger cells compared to identical cells that were not exposed to the micro-current. In some aspects, cells exposed to a micro-current will display a combination of characteristics (e.g., both increased metabolite production and increased cell size, or increased metabolite production and a greater number of CFUs, or some combination thereof). In another aspect, different cell types (e.g., E. coli and S. cerevisiae) with different modifications (e.g., wild type, modified with a DNA construct as disclosed herein, etc.) will respond to different levels of micro-current with different growth increases.

In one aspect, cells exposed to a micro-current will display from greater than 1 to about 5 times more CFUs than identical cells not exposed to a micro-current, or about 1.1, 1.5, 2, 2.5, 3, 3.5, 4, 4.5, or about 5 times more CFUs, or a combination of any of the foregoing values, or a range encompassing any of the foregoing values. In another aspect, cells exposed to a micro-current will display about 1.1, 1.16, 1.27, 1.38, 1.41, or about 1.5 times more CFUs than identical cells not exposed to a micro-current.

In one aspect, S. cerevisiae cells containing DNA constructs capable of producing NAD(P) or NAD(P)H as disclosed herein form more CFUs when exposed to a 120 mV or a 300 mV micro-current.

### Metabolite Production

In one aspect, metabolite production increases when cells capable of producing the metabolite are exposed to a micro-current. In a further aspect, when cells capable of producing NAD(P)/NAD(P)H, as disclosed herein are contacted with a micro-current, the cells produce from greater than 1 to about 5 times more of these metabolites than identical cells not contacted with a micro-current, or from about 1.05, 1.1, 1.5, 2, 2.5, 3, 3.5, 4, 4.5, or about 5 times more of the specified metabolites, or a combination of any of the foregoing values, or a range encompassing any of the foregoing values.

### Methods for Enhancing Microbial Cultures and the Production of Microbial Metabolites

In one aspect, disclosed herein is a method for increasing growth of cells in a microbial culture, the method including at least the step of contacting the microbial culture with a disclosed lysate or extract. In a further aspect, presence of NAD(P)H and/or NAD(P)+ in a microbial culture can increase production of one or more desired metabolites, or can increase a count of microbial cells in a culture dish or vessel. In an aspect, the microbial culture can be a bacterial culture or a fungal culture.

In a further aspect, the compositions and extracts disclosed herein are useful in culturing the following types or organisms: (1) Saccharomyces cerevisiae for use in yeast doughs, brewing beer and wine, genetic research, and production of desirable secondary metabolites including the enzymes invertase and raffinase; Kluyveromyces species for the commercial production of lactase; and Candida species for the commercial production of lipase; (2) Lactobacillus species for use in making fermented foods such as yogurt, kefir, cheese, sauerkraut, pickles, hard cider, wine, and beer as well as for the commercial production of lactic acid and engineered Lactobacillus species engineered to produce protein drugs such as, for example, insulin; (3) Pyrococcus furiosus, Thermus aquaticus, Bacillus stearothermophilus, Thermus filiformis, Thermus thermophilus, and other thermophiles for production of heat stable polymerases for use in the polymerase chain reaction (PCR); (4) Xanthomonas species for production of xanthan gum, used in a variety of food and cosmetic products; (5) Aspergillus niger, used in the production of citric acid and fermentation of sake and other alcoholic beverages and this and other Aspergillus species for commercial production of α-amylase, aminoacylase, glucoamylase, catalase, glucose oxidase, lactase, pectinase, pectin lyase, and protease; Trichoderma species for the commercial production of cellulose; Mucor miehei for the commercial production of rennet; Rhizopus species for the commercial production of lipase; and Mortierella species for the commercial production of raffinase; (6) Clostridium species for production of botulinum toxin for cosmetic and medical purposes as well as the production of butanol (i.e., from Clostridium acetobutylicum); (7) Streptomyces species for production of antibiotics, antiparasitic, antineoplastic, and antifungal compounds including, but not limited to, chloramphenicol, daptomycin, fosfomycin, lincomycin, neomycin, nourseothricin, puromycin, streptomycin, tetracycline, oleandomycin, tunicamycin, mycangimycin, boromycin, bambermycin, clavulanic acid, guadinomine, ivermectin, migrastatin, bleomycin, erythromycin, geldanamycin, and the like; (8) Penicillium species for the production of penicillin and other beta-lactam antibiotics and precursors to semi-synthetic beta-lactam antibiotics; (9) Acetobacter aceti for production of acetic acid; (10) Bacillus species for commercial production of α-amylase, β-amylase, glucose isomerase, penicillin amidase, and protease; E. coli for commercial production of asparaginase; and Klebsiella species for commercial production of pullulanase; (11) Trichoderma polysporum for the production of cyclosporine A, (12) yeasts such as Monascus purpureus for the production of statin drugs for lowering blood cholesterol; (13) Bacillus thuringiensis for the production of insecticides, and other commercially important bacteria, fungi, algae, and cyanobacteria.

### Methods for Enhancing the Physiological Properties of Plants and the Production of Plant Metabolites

The compositions and extracts described herein can enhance the physiological properties of a plant. The term “physiological property” as defined herein includes any physical, chemical, or biological feature that is improved using the compositions and extracts described herein. In one aspect, the compositions and extracts can enhance the growth rate of the plant.

In one aspect, in plants, NAD(P)H and NAD(P)+ balance is responsible for maintaining the redox state in cell compartments. In another aspect, NAD(P)/H is important in cell signaling and various metabolic pathways including, but not limited to, lipid synthesis and carbon and nitrogen metabolism.

In still another aspect, NAD(P)/H is important to seed germination as well as root development and aspects of plant reproduction including floral transition. In yet another aspect, NAD(P)/H plays a role in immunity to pathogens and stress tolerance in plants.

In any of these aspects, contacting a plant, seed, or plant callus with the disclosed lysates and extracts can expose the plant to NAD(P)H and/or NAD(P)+ which can enhance various aspects of the plant's metabolism including growth, nitrogen fixation, carbon fixation, production of metabolites, and the like.

Herein, “plant” is used in a broad sense to include, for example, any species of woody, ornamental, crop, cereal, fruit, or vegetable plant, as well as photosynthetic green algae. “Plant” also refers to a plurality of plant cells that are differentiated into a structure that is present at any stage of the plant's development. Such structures include, but are not limited to, fruits, shoots, stems, leaves, flower petals, roots, tubers, corms, bulbs, seeds, gametes, cotyledons, hypocotyls, radicles, embryos, gametophytes, tumors, and the like. “Plant cell,” “plant cells,” or “plant tissue” as used herein refer to differentiated and undifferentiated tissues of plants including those present in any of the tissues described above, as well as to cells in culture such as, for example, single cells, protoplasts, embryos, calluses, etc.

The selection of the plant used in the methods described herein can vary depending on the application. For example, a specific plant can be selected that produces certain desirable metabolites. Current techniques for producing most plant metabolites are expensive. For example, large amounts of fresh plant biomass must be cultivated and harvested, and expensive and time-consuming extraction methods must be used. The compositions and extracts described herein enhance the production of metabolites from plants that naturally produce these metabolites. Other physiological properties can also be enhanced through use of the disclosed methods including, but not limited to, leaf area, root length, growth rate, immunity to pathogens, stress tolerance, fruit size, fruit number, production of a plant metabolite, or any combination thereof.

In one aspect, plant cells when contacted with the compositions and extracts described herein exhibit enhanced production of various desirable metabolites. Recipient cell targets include, but are not limited to, meristem cells, Type I, Type II, and Type III callus, immature embryos and gametic cells such as microspores, pollen, sperm, and egg cells. It is contemplated that any cell from which a fertile plant may be regenerated is useful as a recipient cell. Type I, Type II, and Type III callus may be initiated from tissue sources including, but not limited to, immature embryos, immature inflorescences, seedling apical meristems, microspores, and the like. Those cells that are capable of proliferating as callus are also useful herein. Methods for growing plant cells are known in the art. In one aspect, plant calluses grown from 2 to 4 weeks can be used herein. The plant cells can also be derived from plants varying in age. For example, plants that are 80 days to 120 days old after pollination can be used to produce calluses useful herein.

The plant cells can be contacted with the compositions and extracts described herein in a number of different ways. In one aspect, the compositions and extracts described herein can be added to media containing the plant cells or can be the media containing the plant cells. In another aspect, the compositions and extracts can be injected into the plant cells via syringe. The amount of extract and the duration of exposure to the extract can vary as well.

Once the plant cells have been in contact with the compositions and extracts for a sufficient time to produce a desired metabolite, the metabolite is isolated. In one aspect, the metabolite is extracted from the media containing the plant cells. The selection of extraction solvent can vary depending on the solubility of the metabolite.

In other aspects, the compositions and extracts described herein can increase the growth rate of a plant. In particular, the compositions and extracts described herein are effective in accelerating plant development in the early stages of tissue culturing. By accelerating plant development in the early stages, it is possible to harvest more metabolites from the plant. Additionally, traditional methods for tissue culture involve the use of synthetic growth factors such as 2,4-dichlorophenoxyacetic acid (2,4-D), which can pose environmental concerns. The compounds and extracts described herein avoid the need for such compounds.

In certain aspects, any of the compositions and extracts described herein can be used in combination with a polysaccharide to enhance one or more physiological properties of the plant. In one aspect, the plant cells are first contacted with the compositions and extracts, then subsequently contacted with the polysaccharide. In another aspect, the plant cells are first contacted with the polysaccharide, then subsequently contacted with the compositions or extracts. In a still further aspect, the plant cells are contacted simultaneously with the polysaccharide and the compositions and extracts.

In one aspect, the polysaccharide includes chitosan, glucosamine (GlcN), N-acetylglucosamine (NAG), or any combination thereof. Chitosan is generally composed of GlcN and NAG units and can be chemically or enzymatically extracted chitin, which is a component of arthropod exoskeletons and fungal and microbial cell walls. In certain aspects, the chitosan can be acetylated to a specific degree of acetylation in order to enhance tissue growth during culturing as well as metabolite production. In one aspect, chitosan isolated from shells of crab, shrimp, lobster, and/or krill is useful herein. In one aspect, the chitosan is in a solution of water and acetic acid at less than 1% by weight, less than 0.75% by weight, less than 0.5% by weight, less than 0.25% by weight, or less than 0.1% by weight. In another aspect, the amount of chitosan that is applied to the plant cells is from 0.1% to 0.01% by weight, from 0.075% to 0.025% by weight, or is about 0.05% by weight. The polysaccharides used herein are generally natural polymers and thus present no environmental concerns. Additionally, the polysaccharides can be used in acceptably low concentrations. In certain aspects, however, the polysaccharides can be used in combination with one or more plant growth regulators.

In one aspect, the plant growth regulator is an auxin, a cytokinin, a gibberellin, abscisic acid, or a polyamine. In a further aspect, the auxin is a natural or synthetic auxin. In a still further aspect, the auxin is indole-3-acetic acid (IAA), 4-chloroindole-3-acetic acid (4-CI-IAA), 2-phenylacetic acid (PAA), indole-3-butyric acid (IBA), 2,3-dichlorophenoxyacetic acid (2,4-D), α-naphthalene acetic acid (α-NAA), 2-methoxy-3,6-dichlorobenzoic acid (dicamba), 4-amino-3,5,6-trichloropicolinic acid (torden or picloram), 2,4,5-trichloropicolinic acid (2,4,5-T), or a combination thereof. In another aspect, the cytokinin is zeatin, kinetin, 6-benzylaminopurine, diphenylurea, thidizuron (TDZ), 6-(γ,γ-dimethylallylamino) purine, or a combination thereof. In another aspect, the gibberellin is gibberellin A1 (GA1), gibberellic acid (GA3), ent-gibberellane, ent-kaurene, or a combination thereof. In yet another aspect, the polyamine is putrescine, spermidine, or a combination thereof.

In one aspect, the plant cell or callus is first contacted with a polysaccharide and subsequently contacted with a plant growth regulator. In another aspect, the plant cell or callus is first contacted with a plant growth regulator and subsequently contacted with a polysaccharide. In an alternative aspect, the plant cell or callus is simultaneously contacted with a polysaccharide and a plant growth regulator. In a further aspect, the plant cell or callus is only contacted with a polysaccharide and is not contacted with a plant growth regulator.

The plant cells can be contacted with the polysaccharide using a number of techniques. In one aspect, the plant cells or reproductive organs (e.g., a plant embryo) can be cultured in agar and medium with a solution of the polysaccharide. In other aspects, the polysaccharide can be applied to a plant callus by techniques such as, for example, coating the callus or injecting the polysaccharide into the callus. In this aspect, the age of callus can vary depending on the type of plant. The amount of polysaccharide can vary depending upon, among other things, the selection and number of plant cells. The use of the polysaccharide in the methods described herein permits rapid tissue culturing at room temperature. Due to the ability of the polysaccharide to prevent microbial contamination, the tissue can grow for extended periods of time ranging from days to several weeks. Moreover, tissue culturing with the polysaccharide can occur in the dark and/or light. As discussed above, the plant cells are also contacted with any of the compositions or extracts described above. Thus, the use of the polysaccharides and compositions and extracts described herein is a versatile way to culture and grow plant cells—and, ultimately, plants of interest—with enhanced physiological properties.

In other aspects, the plant cells can be cultured in a liquid medium on a larger scale in a bioreactor. For example, plant cells can be cultured in agar and mediu9m, then subsequently contacted with the compositions and extracts described herein. After a sufficient culturing time (e.g., two to four weeks), the plant cells are introduced into a container with the same medium used above and, additionally, the polysaccharide. In certain aspects, the polysaccharide can be introduced with anionic polysaccharides including, but not limited to, alginates (e.g., sodium alginate, calcium alginate, potassium alginate, etc.). After the introduction of the polysaccharide, if using, the solution is mixed for a sufficient time to produce a desired result (e.g., production of a desired metabolite). Alternatively, the initial liquid medium in the bioreactor can include any of the compositions and/or extracts described herein.

In one aspect, provided herein is a plant grown by the process that involves contacting plant gamete cells or a plant reproductive organ with the compositions and extracts disclosed herein. In a further aspect, the plant is produced by the following method:
(a) contacting a plant callus with the compositions and extracts;
(b) culturing the plant callus; and
(c) growing the plant from the plant callus.

In a further aspect, the same method can be applied to other plant parts including fruits, stems, roots, tubers, corms, bulbs, flowers, buds, seeds, and the like. In a still further aspect, the same method can be applied to an entire plant.

In one aspect, the plant callus is immersed in a solution of polysaccharide (e.g., chitosan), then inoculated with the compositions and/or extracts. In another aspect, the plant callus can be from 2 days up to 20 days old prior to inoculation with the compositions and/or extracts described herein. The plant callus is then allowed to grow until it is of sufficient weight and size. In one aspect, the plant callus is allowed to grow (i.e., culture) for 1 to 10 weeks after inoculation. Following growth or culture of the callus for a sufficient period of time, desired metabolites can be collected according to methods known in the art; said methods are specific to the desired metabolites and make use of properties ranging from molecular size to charge to hydrophobicity or hydrophilicity to other properties useful for collection and purification of the metabolites.

### Oral Dosage Forms

In various aspects, the present disclosure relates to nutritional compositions comprising a at least one product of a disclosed method (e.g. an extract or lysate from a biological device as described herein). In some aspects, the nutritional compositions can include pharmaceutically acceptable carriers. As used herein, “pharmaceutically-acceptable carriers” means one or more of a pharmaceutically acceptable diluents, preservatives, homogenizers, antioxidants, solubilizers, emulsifiers, coloring agents, releasing agents, coating agents, sweetening, flavoring and perfuming agents, and adjuvants. The disclosed nutritional compositions can be conveniently presented in unit dosage form and prepared by any of the methods well known in the art of pharmacy and pharmaceutical sciences.

In a further aspect, the disclosed nutritional compositions comprise at least one disclosed extract or lysate and/or at least one product of a disclosed method and a pharmaceutically acceptable carrier. The disclosed nutritional compositions include those suitable for oral administration. In various aspects, the present disclosure also relates to a nutritional composition comprising a pharmaceutically acceptable carrier or diluent.

In practice, the compositions of the present disclosure can be combined as the active ingredient in intimate admixture with a pharmaceutical carrier according to conventional pharmaceutical compounding techniques. Thus, the nutritional compositions of the present disclosure can be presented as discrete units suitable for oral administration such as capsules, cachets or tablets each containing a predetermined amount of the nutritional composition. Further, the compositions can be presented as a powder, as granules, as a solution, as a suspension in an aqueous liquid, as a non-aqueous liquid, as an oil-in-water emulsion or as a water-in-oil liquid emulsion.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the composition to the patient can open a single container or package with the entire dose contained therein, and does not have to mix any components together from two or more containers or packages. Typical examples of unit dosage forms are tablets (including scored or coated tablets), capsules or pills for oral administration; powder packets; wafers; and segregated multiples thereof. This list of unit dosage forms is not intended to be limiting in any way, but merely to represent typical examples of unit dosage forms.

The pharmaceutical compositions disclosed herein comprise a nutritional composition of the present disclosure as an active ingredient, a pharmaceutically acceptable carrier, and optionally one or more additional vitamins or similar agents. In one aspect, the pharmaceutical compositions can be available by prescription. In an alternative aspect, the pharmaceutical compositions are available over the counter. In some aspects, the pharmaceutical compositions additionally have one or more of an anti-inflammatory property, an anti-itch property, a hair growth property, or any combination thereof. In one aspect, the pharmaceutical compositions can be stored at room temperature or can be refrigerated or frozen prior to use.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical or food-grade diluents, excipients, extenders, or carriers (termed herein as a pharmaceutically acceptable carrier, or a carrier) suitably selected with respect to the intended form of administration and as consistent with conventional pharmaceutical practices. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent advantageous oral dosage unit forms in which case solid pharmaceutical carriers are obviously employed. However, other dosage forms may be suitable depending upon clinical population (e.g., age and severity of clinical condition), solubility properties of the specific disclosed composition used, and the like. Accordingly, the disclosed lysates and extracts can be used in oral dosage forms such as pills, powders, granules, elixirs, tinctures, suspensions, syrups, sublingual dissolvable strips, hydrogel microparticles, and emulsions, capsules, tablets, buccal strips, troches, functional foods, powders, pellets, and beverages. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate,-phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate,-triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate,-dibutyrate,-dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a lysate or extract that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed lysate or extract can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22: 42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl) imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In some aspects, the oral dosage form can be a rapidly dissolving film (RDF) or oral thin film (OTF) to be placed in an area of the mouth such as the sublingual area or the buccal cavity. In a further aspect, such films can include strip-forming polymers, plasticizers, sweetening and/or flavoring agents, coloring agents, stabilizers, thickeners, permeation enhancers, disintegrants, saliva stimulating agents, and the like. In a further aspect, these ingredients are already approved for use in other oral pharmaceutical dosage forms such as those described above. In other aspects, the oral dosage form can be a fast-dissolving tablet or lozenge or the like. In one aspect, the strip-forming polymers can be hydrophilic polymers and can be prepared into strip form by solvent casting and/or hot-melt extrusion. In one aspect, RDF or OTF formulations can have a large surface area for fast disintegration and absorption of active ingredients. In a still further aspect, the film formulations may adhere to the oral cavity by a method such as, for example, interaction with saliva, such that the film does not become displaced in the oral cavity and/or accidentally swallowed. In an aspect, the stabilizer can be ethanol, n-propanol, glycerol, a polyethylene glycols with a molecular weights between 200 Da and 600 Da, diethylene glycol monoethyl ether, 1,2-propylene glycol, urea, urethane, acetamide, N-methyl acetamide, N, N-diethyl acetamide, N,N-dimethyl acetamide, ethylenediamine, hydroxyethyl theophylline, tromethamine, an aliphatic amino acid, or any combination thereof. In some aspects, a component can act in more than one capacity (e.g. propylene glycol can be a stabilizer, a permeation enhancer, a plasticizer, or any combination thereof.

Exemplary components of oral thin films include, but are not limited to, the following. In one aspect, the water soluble strip-forming polymer can be selected from hydroxypropyl methylcellulose (HPMC) E3, HPMC E5, HPMC E15, HPMC K-3, methylcellulose A-3, methylcellulose A-6, methylcellulose A-15, pullulan, carboxmethylcellulose or a derivative thereof, polyvinylpyrollidone (PVP) K-90, pectin, gelatin. sodium alginate, hydroxypropylcellulose, polyvinyl alcohol, maltodextrins, calcium alginate, a polyactive carbohydrate, chitosan, or any combination thereof. In one aspect, polyactive carbohydrates are further described in U.S. Pat. Nos. 10,995,353 and 12,129,500. In some aspects, strip-forming polymers can also serve as disintegrants, or a separate disintegrant can be added, such as, for example, cross-linked polyvinyl pyrrolidone, sodium carboxymethyl starch, sodium carboxymethyl cellulose, microcrystalline cellulose, or any combination thereof. In one aspect, suitable permeation enhancers include, but are not limited to, 2,3-lauryl ether, aprotinin, azone, benzalkonium chloride, cetylpyridinium chloride, cetyltrimethyl ammonium bromide, cyclodextrin, dextran sulfate, lauric acid, lysophosphatidylcholine, menthol, phosphatidylcholine, polyoxyethylene, polysorbate 80, propylene glycol, disodium ethylenediaminetetraacetic acid (EDTA), sodium glycocholate, sodium glycodeoxycholate, sodium lauryl sulfate, sodium salicylate, sodium taurocholate, sodium taurodeoxycholate, or any combination thereof. In another aspect, suitable plasticizers include glycerol, dibutyl phthalate, polyethylene glycol, and combinations thereof. In still another aspect, useful surfactants include sodium lauryl sulfate, benzalkonium chloride, polysorbates, and combinations thereof, while useful sweeteners include saccharin, cyclamate, aspartame, steviol glycosides, or combinations thereof. In a still further aspect, saliva stimulating agents, which may also enhance flavor, include, but are not limited to, citric acid, malic acid, lactic acid, ascorbic acid, and combinations thereof. In an aspect, ascorbic acid may be preferred since it is a useful vitamin. (i.e., vitamin C) in nutritional compositions. In some aspects, for oral thin films and/or rapidly dissolving films, the following general amounts of components may be used: active ingredient or lysate/extract: 5-30% by weight, polymer: 45% by weight, plasticizer: 0-20% by weight, sweetener: 3-6% by weight, and saliva stimulating agent: 2-6% by weight. However, amounts of components can be varied depending on the formulation and desired effect and should thus also be considered disclosed.

In another aspect, the nutritional supplements and oral dosage forms can be provided as hydrogel microparticles. In a further aspect, the hydrogel microparticles can include one or more hydrogel forming materials such as, for example, sodium alginate, calcium alginate, a polyactive carbohydrate, chitosan, glucosamine, chondroitin, or any combination thereof. In one embodiment, the hydrogel microparticles can include chitosan (1% w/v) in acetic acid 3% (w/v) and sodium alginate (2% w/v). Further in this aspect, to produce the hydrogels, one or more of sodium hydroxide (NaOH) and/or calcium chloride (CaCl₂)) can be added to the hydrogel forming materials. In an aspect, the hydrogel microparticles can encapsulate the active compounds (proteins, vitamins, and the like) from the disclosed lysates and extracts and may facilitate absorption of the compounds, delivery of the compounds, or the like.

In some aspects, any of the above-listed oral dosage forms can be formulated for addition to food or beverage products, either by incorporating the dosage forms into the food or beverage products at the time of manufacture, or being added by the consumer to the food or beverage products. In a further aspect, the disclosed lysates and extracts can be provided as freeze-dried, spray-dried, or other powders for incorporation into foods and beverages by the consumer or in manufacturing facilities. In an aspect, the disclosed lysates and extracts and nutritional compositions formed therefrom can be formulated as a food additive, a food seasoning, a flavor enhancer, or a component thereof. In another aspect, the disclosed lysates and extracts can be stored at room temperature or can be refrigerated or frozen prior to use.

In another aspect, the disclosed oral dosage forms can further include one or more additional vitamins or minerals. In a further aspect, the additional vitamins can be selected from all-trans-retinols, all-trans-retinyl-esters, all-trans-β-carotene another provitamin A carotenoid, thiamine, riboflavin, niacin, niacinamide, pantothenic acid, pyridoxine, biotin, folic acid another folates, a cobalamin, ascorbic acid, a calciferol, a tocopherol, a tocotrienol, a phylloquinone, a menaquinone, a menadione, choline, nicotinamide adenine dinucleotide, nicotinamide adenine dinucleotide phosphate, or any combination thereof. In a further aspect, the additional minerals can be selected from calcium, chloride, magnesium, phosphate, potassium, sodium, chromium, copper, fluoride, iodine, iron, manganese, molybdenum, selenium, zinc, or any combination thereof.

### Additional Dosage Forms

In some aspects, the lysates and extracts disclosed herein can be administered to a subject in need thereof in a form other than an oral dosage form. In one aspect, such dosage forms include, but are not limited to, administration that is topical, intravenous, subcutaneous, injectable, intranasal, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In various aspects, the present disclosure relates to pharmaceutical compositions comprising a therapeutically effective amount of at least one lysate or extract, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed lysate or extract, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed lysate or extract or a product of a disclosed method of making. In a further aspect, a disclosed lysate or extract or a product of a disclosed method of making may be formulated into various pharmaceutical forms for administration purposes.

The lysates and extracts described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable for injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed lysates and/or extracts can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a lysate or extract of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In one aspect, formulas for topical administration can include, but are not limited to, the following: a cream, ointment, lotion, spray, soap, nail treatment, body wrap, face mask, swab, lip balm, transdermal patch, scalp treatment, aftershave lotion, or any combination thereof. In a further aspect, formulas for topical administration can be applied to the skin, scalp, fingernails, toenails, hair, mucous membranes, or any combination thereof. In one aspect, formulas for topical administration can be applied to the eye in the form of eye drops, ointment, or gel.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds. In one aspect, formulations for rectal delivery include suppositories, enemas, and/or creams, while formulations suitable for vaginal delivery may include suppositories or creams.

Pharmaceutical compositions containing a lysate or extract of the present disclosure can also be prepared in powder or liquid concentrate form.

In one aspect, pharmaceutical compositions for administration via the pulmonary route (e.g., by inhalation), including by nebulization, according to the present disclosure can include compounds useful for adjusting a solution's osmotic pressure (tonicity). In a further aspect, these compounds can include, but are not limited to, sodium chloride, dextrose, and combinations thereof. In some aspects, these compounds adjust osmotic pressure to a range of from about 300 to about 700 mOsmol/kg, or about 300, 350, 400, 450, 500, 550, 600, 650, or about 700 mOsmol/kg, or a combination of any of the foregoing values, or a range encompassing any of the foregoing values.

In another aspect, the pharmaceutical compositions can include compounds useful for adjusting solution pH. Without wishing to be bound by theory, a liquid medication, whether inhaled or injected, may work best when the carrier has a pH similar to physiological conditions. In another aspect, pH adjustments may also enhance drug stability and/or drug solubility. In one aspect, pH can be adjusted by adding a compound such as sodium hydroxide or potassium hydroxide (to increase the pH and/or made the carrier more basic) or hydrochloric acid or sulfuric acid (to decrease the pH and/or make the carrier more acidic). In another aspect, pH can be adjusted using a biocompatible buffer such as, for example, 2-(N-morpholino) ethanesulfonic acid (MES), bis-tris methane, N-(2-acetamido)-2-iminodiacetic acid (ADA), N-(2-acetamido)-2-aminoethanesulfonic acid (ACES), bis-tris propane, piperazine-N,N′-bis(2-ethanesulfonic acid (PIPES), N-(2-acetamido)-2-aminoethanesulfonic acid (ACES), 2-hydroxy-3-morpholinopropanesulfonic acid (MOPSO), cholamine chloride, 3-(N-morpholino) propanesulfonic acid (MOPS), N,N-bis(2-hydroxyethyl)-2-aminoethanesulfonic acid (BES), 2-[(2-hydroxy-1,1-bis(hydroxymethyl)ethyl)amino]ethanesulfonic acid (TES), 4-(2-hydroxyethyl)-1-piperazineethanesulfonic acid (HEPES), 3-(N,N-bis[2-hydroxyethyl]amino)-2-hydroxypropanesulfonic acid (DIPSO), 4-(N-morpholino) butanesulfonic (MOBS), acetamidoglycine, 2-hydroxy-3-[tris(hydroxymethyl)methylamino]-1]propanesulfonic acid (TAPSO), triethylammonium acetate (TEAA), piperazine-1,4-bis(2-hydroxypropanesulfonic acid)dihydrate (POPSO), N-(hydroxyethyl) piperazine-N′-2-hydroxypropanesulfonic acid (HEPPSO), 4-(2-hydroxyethyl)-1-piperazinepropanesulfonic acid (EPPS), tricine, tris, glycinamide, glycylglycine, N-(2-hydroxyethyl) piperazine-N′-(4-butanesulfonic acid) (HEPBS), bicine, [tris(hydroxymethyl)methylamino]propanesulfonic acid (TAPS), N-cyclohexyl-2-aminoethanesulfonic acid (CHES), aminomethyl propanol (AMP), N-(1,1-dimethyl-2-hydroxyethyl)-3-amino-2-hydroxypropanesulfonic acid (AMPSO), N-cyclohexyl-2-hydroxyl-3-aminopropanesulfonic acid (CAPSO), N-cyclohexyl-3-aminopropanesulfonic acid (CAPS), 4-(cyclohexylamino)-1-butanesulfonic acid (CABS), citrate buffer, phosphate buffer, a sodium salt thereof, or a combination thereof. In some aspects, a pH of from about 4.5 to about 6.5 may be useful to prevent sneezing or respiratory irritation. In another aspect, pH may also be important to product stability during transport and/or storage.

In one aspect, the formulations can be purged with an inert gas such as, for example, nitrogen, during the packaging process. In one aspect, purging may help prevent or reduce oxidation, thus extending the shelf life of the pharmaceutical compositions. In another aspect, the formulations can include a preservative such as, for example, benzalkonium chloride or another quaternary ammonium compound, ethanol, propylene glycol, benzoyl alcohol, chlorobutanol, methylparaben or another paraben, or a combination thereof. In any of these aspects, the preservative can reduce or prevent microbial growth in the pharmaceutical compositions. In still another aspect, the pharmaceutical compositions can include a surfactant such as, for example, polysorbate 20, polysorbate 80, or another surfactant. In another aspect, the surfactant can increase suspendability of the active ingredient and/or stability of suspensions including the active ingredient. In some aspects, the formulations can include a chelating agent such as, for example, disodium EDTA, which can, in a further aspect, increase the stability of the formulations. In one aspect, the formulations can include a suspending agent. In a further aspect, the suspending agent can be carboxymethyl cellulose or sodium carboxymethyl cellulose. In one aspect, the suspending agent can alter the viscosity of the solution. In another aspect, if the active ingredient is administered as a suspension, the suspending agent can enhance the suspendability of the active ingredient and/or the stability of any suspensions formed using the active ingredient. In some aspects, the formulations include a co-solvent such as, for example, alcohol, a polyethylene glycol such as, for example, PEG 400, propylene glycol, or a combination thereof. In another aspect, the co-solvent can increase the solubility of the active ingredient(s). In one aspect, the formulations can include a humectant such as, for example, glycerin. In one aspect, for pharmaceutical compositions intended to be administered by nebulizer, doses can be packaged as unit-dose vials to avoid the need for antimicrobial agents. Further in this aspect, the compositions are sterile.

In another aspect, pharmaceutical compositions administered by the pulmonary route, e.g., by a metered dose inhaler (MDI), can include antioxidants such as, for example, acetone sodium bisulfate, ascorbic acid, or a combination thereof. In another aspect, the pharmaceutical compositions for use in MDIs disclosed herein can include preservatives such as, for example, ammonia, benzalkonium chloride, cetylpyridinium chloride, and other quaternary ammonium compounds, chlorobutanol, methylparaben, propylparaben, and other parabens, sodium metabisulfite, sodium bisulfite, sodium sulfite, thymol, or a combination thereof. In one aspect, the compositions disclosed herein may include agents for adjusting tonicity or osmotic pressure including, but not limited to, sodium chloride, sodium sulfate, and combinations thereof. In one aspect, the formulations may include flavoring agents including, but not limited to, citric acid, menthol, saccharin, saccharin sodium dehydrate, or a combination thereof. In any of these aspects, the formulations can include a chelating agent such as, for example, disodium EDTA. In one aspect, formulations for administration by metered dose inhaler include a cosolvent such as, for example, ethanol, glycerin, propylene glycol, water, or a combination thereof. In one aspect, the formulations disclosed herein include buffering agents and/or pH stabilizers including, but not limited to, glycine, hydrochloric acid, lysine monohydrate, nitric acid, sodium bisulfate, sodium citrate, sodium hydroxide, sulfuric acid, tromethamine, another biocompatible pH stabilizer or buffering agent, or a combination thereof. In one aspect, the excipients in the MDI formulations can include a dispersion or solubilization agent such as, for example, lecithin, magnesium stearate, oleic acid, a polyethylene glycol (e.g., PEG 10000), sorbitan trioleate, carboxylic acid functionalized methyl polyethylene glycol (f-mPEG), oligolectic acid (OLA), a combination thereof, or any of these co-formulated with ethanol. In one aspect, a suspending aid such as, for example, polysorbate 80 or polyvinylpyrrolidone K25 can be included in the formulations disclosed herein. In another aspect, in some formulations disclosed herein, a surfactant can be useful. Further in this aspect, the surfactant can be selected from sorbitan monolaurate, sorbitan monooleate, sorbitan trioleate, a PEG (e.g., PEG300, PEG 600, PEG 1000), a propoxylated PEG, PEG-4 lauryl ether, PEG-23 lauryl ether, PEG-10 cetyl ether, PEG-2 stearyl ether, PEG-10 oleyl ether, polysorbate 20, polysorbate 60, polysorbate 80, a polypropylene glycol (PPG) such as PPG 2000, a block copolymer of PEG and PPG such as, for example, pluronic 10-R5, 17-R2, 17-R4, 25-R4, F-68, F-127, L043, L-44 NF, L-62, L-64, L-101, sodium dioxtyl sulfosuccinate, or a combination thereof. In some aspects, these excipients can have multiple functions, e.g., benzalkonium chloride may aid in wetting and solubilization in addition to acting as a preservative. In one aspect, when the MDI formulation is a suspension, a bulking agent may be included. In one aspect, the bulking agent can be a saccharide such as lactose or maltose, an amino acid such as glycine or leucine, a salt, or a combination thereof.

In one aspect, when preparing the pharmaceutical composition as disclosed herein for administration by an MDI, dry components should be reduced to have an average particle size suitable for administration by MDI. In one aspect, particle size reduction can be achieved by milling (e.g., ball milling or jet milling), spray drying, use of a supercritical fluid, or a combination thereof.

In another aspect, formulations for nasal or inhalable delivery can include swabs, nasal sprays, and/or nebulizers, in addition to metered dose inhalers.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed lysate or extract formulated in a compatible pharmaceutical carrier may also be prepared, placed in an appropriate container, and labeled for treatment of an indicated condition.

The exact dosage and frequency of administration depends on the particular disclosed lysate or extract or a product of a disclosed method of making; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### Veterinary Use and Human Use

In one aspect, disclosed herein is a method for providing nutrition to a subject in need thereof, the method including at least the step of administering a disclosed nutritional composition or pharmaceutical composition to the subject. In a further aspect, the subject can be a mammal such as, for example, a human, non-human primate, cat, dog, rat, mouse, hamster, guinea pig, rabbit, horse, cattle, swine, goat, or sheep. In another aspect, the subject can be a bird such as, for example, a chicken, turkey, duck, or parrot.

Also disclosed herein are cosmetic compositions including the disclosed lysates and extracts in a physiologically acceptable medium. In a further aspect, the cosmetic compositions may be tooth whiteners, lotions, hair conditioners, soaps, deodorants, aftershave lotions, shaving creams, lip balms, face masks, scalp treatments, or a combination thereof.

### Advantages and Applications of the Disclosed Formulations and Compositions

In an aspect, the disclosed formulations and compositions can provide supplemental nutrition to those in need thereof for a variety of medical reasons. In one aspect, patients with neurological or other conditions that impair swallowing or otherwise cause dysphagia (e.g., stroke, amyotrophic lateral sclerosis, and/or Parkinson's disease), forms of supplemental nutrition capable of being sublingually absorbed may be particularly important. In another aspect, individuals with gastrointestinal disease or reduced stomach capacity due to bariatric surgery may benefit from additional vitamins in non-bulky or low-volume forms. In some aspects, cancer patients and others experiencing nausea may benefit from nutrition sources that can be absorbed sublingually. In one aspect, the present compositions and formulations can address each of these needs. In some aspects, however, when the consumer is able to consume bulk foods and beverages but in need of supplemental nutrition, the disclosed compositions can be added to the foods and/or beverages that are already part of the consumer's diet.

In an aspect, one advantage of a sublingual strip or film is that it can be consumed without additional liquid, such as, for example, would be required for swallowing a tablet or capsule. In another aspect, sublingual strips may be advantageous because components thereof can be directly absorbed into the bloodstream. In a further aspect, direct absorption can bypass the gastrointestinal tract and avoid digestion, swallowing or choking difficulties, vomiting or nausea, and the like. In alternative aspects, the strips can be dissolved in the buccal cavity instead of sublingually. However, other oral dosage formulations are contemplated and should be considered disclosed including a mouth rinse, a mouthwash, a toothpaste, a coating for the teeth, a candy, a troche, a powder, a pellet, a chewing gum, a chewable tablet, a gummy candy, or any combination thereof.

In some aspects, individuals with diseases or conditions impacting intestinal absorption (alcohol use disorder, other substance abuse disorders, celiac disease, Crohn's disease, other inflammatory bowel diseases and disorders, and the like) may have trouble absorbing B vitamins and related compounds via the gastrointestinal tract. Thus, in one aspect, NAD compositions produced by the disclosed methods and devices can be delivered by other means such as by injection or intravenously, through a nasal spray or other inhaled mechanism, or the like. In this aspect, NAD can bypass intestinal absorption and be used directly by cells. In one aspect, a person having low NAD levels due to poor intestinal absorption, age, or for other reasons, may experience a reduction in symptoms of neurodegenerative disorders or neurological impairments and enhanced mental function. In a further aspect, healthy NAD levels can help with DNA repair and other vital functions. In still another aspect, healthy NAD levels can lead to increased attention span and memory, can help maintain a healthy weight, can promote immunity, can help promote a healthy metabolism, and other beneficial effects.

In one aspect, the NAD is synthesized in eukaryotic host cells like yeast, which are similar to human cells. Therefore, the NAD in the lysate is structurally and functionally compatible with human cellular and molecular systems. In a further aspect, because the NAD is produced by eukaryotic cells, it exhibit nanoparticles size suitable to be sublingually absorbed and/or digested.

### ASPECTS

The present disclosure can be described in accordance with the following numbered aspects, which should not be confused with the claims.

Aspect 1. A DNA construct comprising the following genetic components:
(a) a gene that encodes nicotinamide phosphoribosyltransferase (NAMPT);
(b) a gene that encodes nicotinamide mononucleotide adenylytransferase (NMNAT);
(c) a gene that encodes nicotinamide riboside kinase 1 (NRK1);
(d) a gene that encodes nicotinamide riboside kinase 2 (NRK2); and
(e) a gene that encodes quinolinate phosphoribosyltransferase (QPRT).

Aspect 2. The DNA construct of aspect 1, wherein the gene that encodes the NAMPT has SEQ ID NO. 1 or at least 70% homology thereto.

Aspect 3. The DNA construct of aspect 1 or 2, wherein the gene that encodes NMNAT has SEQ ID NO. 2 or at least 70% homology thereto.

Aspect 4. The DNA construct of any one of aspects 1-3, wherein the gene that encodes NRK1 has SEQ ID NO. 3 or at least 70% homology thereto.

Aspect 5. The DNA construct of any one of aspects 1-4, wherein the gene that encodes NRK2 has SEQ ID NO. 4 or at least 70% homology thereto.

Aspect 6. The DNA construct of any one of aspects 1-5, wherein the gene that encodes QPRT has SEQ ID NO. 5 or at least 70% homology thereto.

Aspect 7. The DNA construct of any one of aspects 1-6, wherein the construct further comprises at least one promoter.

Aspect 8. The DNA construct of aspect 7, wherein the at least one promoter is a T3 promoter, a T7 promoter, an iron promoter, an araBAD promoter, a GAL1 promoter, or any combination thereof.

Aspect 9. The DNA construct of aspect 8, wherein the at least one promoter is GAL1 promoter, and the GAL1 promoter is positioned before the gene that encodes NAMPT, the gene that encodes NMNAT, the gene that encodes NRK1, the gene that encodes NRK2, the gene that encodes QPRT, or any combination thereof.

Aspect 10. The DNA construct of any one of aspects 1-9, wherein the DNA construct further comprises a gene that confers resistance to an antibiotic.

Aspect 11. The DNA construct of aspect 10, wherein the antibiotic comprises tetracycline, neomycin, kanamycin, ampicillin, hygromycin, chloramphenicol, amphotericin B, bacitracin, carbapenem, cephalosporin, ethambutol, fluoroquinolones, isoniazid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

Aspect 12. The DNA construct of any one of aspects 1-11, wherein the DNA construct further comprises at least one terminator.

Aspect 13. The DNA construct of aspect 12, wherein the at least one terminator is an CYC1 terminator.

Aspect 14. The DNA construct of any one of aspects 1-13, wherein the construct comprises from 5′ to 3′ the following genetic components in the following order: (a) a gene that encodes NAMPT; (b) a gene that encodes NMNAT; (c) a gene that encodes NRK1; (d) a gene that encodes NRK2; and (e) a gene that encodes QPRT.

Aspect 15. The DNA construct of any one of aspects 1-14, wherein the construct comprises from 5′ to 3′ the following genetic components in the following order: (a) a gene that encodes NAMPT having SEQ ID NO. 1 or at least 70% homology thereto; (b) a gene that encodes NMNAT having SEQ ID NO. 2 or at least 70% homology thereto; (c) a gene that encodes NRK1 having SEQ ID NO. 3 or at least 70% homology thereto; (d) a gene that encodes NRK2 having SEQ ID NO. 4 or at least 70% homology thereto; and (e) a gene that encodes QPRT having SEQ ID NO. 5 or at least 70% homology thereto.

Aspect 16. The DNA construct of any one of aspects 1-15, wherein the construct comprises from 5′ to 3′ the following genetic components in the following order: (a) a gene that encodes NAMPT, (b) a CYC1 terminator, (c) a GAL1 promoter, (d) a gene that encodes NMNAT, (e) a CYC1 terminator, (f) a GAL1 promoter, (g) a gene that encodes NRK1, (h) a CYC1 terminator, (i) a GAL1 promoter, (j) a gene that encodes NRK2; (k) a CYC1 terminator; (l) a GAL1 promoter; and (m) a gene that encodes QPRT.

Aspect 17. The DNA construct of any one of aspects 1-16, wherein the construct comprises from 5′ to 3′ the following genetic components in the following order: (a) a gene that encodes NAMPT having SEQ ID NO. 1 or at least 70% homology thereto, (b) a CYC1 terminator, (c) a GAL1 promoter, (d) a gene that encodes NMNAT having SEQ ID NO. 2 or at least 70% homology thereto, (e) a CYC1 terminator, (f) a GAL1 promoter, (g) a gene that encodes NRK1 having SEQ ID NO. 3 or at least 70% homology thereto, (h) a CYC1 terminator, (i) a GAL1 promoter, (j) a gene that encodes NRK2 having SEQ ID NO. 4 or at least 70% homology thereto; (k) a CYC1 terminator; (l) a GAL1 promoter; and (m) a gene that encodes QPRT having SEQ ID NO. 5 or at least 70% homology thereto.

Aspect 18. The DNA construct of any one of aspects 1-17, wherein the DNA construct has SEQ ID NO. 6.

Aspect 19. A vector comprising the DNA construct of any one of aspects 1-18.

Aspect 20. The vector of aspect 19, wherein the vector is a plasmid.

Aspect 21. The vector of aspect 20, wherein the plasmid is pWLneo, pSV2cat, pOG44, pXT1, pSG, pSVK3, pBSK, pBSKII, pYES, pYES2, pET, pBAD, pUC, or pUC19.

Aspect 22. The vector of aspect 20, wherein the plasmid is pYES2.

Aspect 23. A biological device comprising host cells transformed with the DNA construct in any one of aspects 1-22.

Aspect 24. The biological device of aspect 23, wherein the host cells comprise fungi.

Aspect 25. The biological device of aspect 24, wherein the fungi comprise Saccharomyces cerevisiae.

Aspect 26. An extract produced by culturing the biological device of any one of aspects 23-25 in a culture medium, wherein the extract comprises NAD, NADH, NADP, NADPH, or any combination thereof

Aspect 27. The extract of aspect 26, wherein the host cells are lysed to produce a lysate.

Aspect 28. The extract of aspect 26 or 27, wherein the cells are exposed to a micro-current during the culturing of the cells.

Aspect 29. The extract of any one of aspects 26-28, wherein the culture medium comprises Dulbecco's Modified Eagle Medium (DMEM), RPMI 1640, Minimal Essential Medium (MEM), Eagle's Minimal Essential Medium (EMEM), Iscove's Modified Dulbecco's Medium (IMDM), DMEM/F12 Medium, Murashige and Skoog (MS) medium, White's medium, Agrobacterium minimal medium, Banana AGS basal medium, Blaydes basal medium, Bold's basal medium, Chu (N6) medium, De Greef and Jacobs Medium, DKW basal medium, Economou and Read basal medium, Gamborg (B5) medium, Gresshoff and Doy medium, Heller medium, Hoagland complete medium, Jensen's medium, Kao and Michayluk medium, Litvay medium, NB basal medium, Nitsch medium. NLN medium, Quoirin and Lepoivre medium, Schenk and Hildebrandt medium, TAP medium, TM4G medium, Vacin and Went medium, wheat callus induction medium, Luria Bertani (LB) broth, terrific broth, tryptic soy broth, minimal salts (M9) medium, SOB medium, SOC medium, yeast malt medium, YPD broth, YNB broth, synthetic complete (SC) medium, YPG medium, Hartwell's complete (HC) medium, or a combination thereof.

Aspect 30. The extract of any one of aspects 26-28, wherein the culture medium comprises Luria Bertani (LB) broth or yeast malt medium.

Aspect 31. The extract of any one of aspects 26-30, wherein the culture medium comprises supplemental vitamins, nucleosides, nucleotides, amino acids, a carbohydrate, an antibiotic, or a combination thereof.

Aspect 32. The extract of any one of aspects 26-31, wherein the culture medium comprises a liquid.

Aspect 33. The extract of aspect 32, wherein the host cells are cultured in a biofermenter.

Aspect 34. The extract of any one of aspects 26-31, wherein the host cells are distributed on a substrate.

Aspect 35. The extract of aspect 34, wherein the substrate comprises agar.

Aspect 36. The extract of any one of aspects 28-35, wherein the host cells are exposed to the micro-current using at least one electrode.

Aspect 37. The extract of aspect 36, wherein the at least one electrode comprises copper, graphite, carbon nanotubes, graphene, titanium, brass, silver, platinum, palladium, iron, nickel, lead, steel, magnesium, aluminum, tin, zinc, tungsten, mixed metal oxides, a spinel-type structure, an olivine-type structure, or a combination thereof.

Aspect 38. The extract of aspect 36, wherein the at least one electrode comprises platinum.

Aspect 39. The extract of any one of aspects 28-38, wherein the micro-current is from about 50 mV to about 300 mV.

Aspect 40. The extract of any one of aspects 28-38, wherein the micro-current is about 120 mV.

Aspect 41. The extract of any one of aspects 28-38, wherein the micro-current is about 200 mV.

Aspect 42. The extract of any one of aspects 28-41, wherein cell growth is greater than 1 to about 5 times higher for the host cells exposed to the micro-current compared to otherwise identical cells not exposed to the micro-current.

Aspect 43. The extract of aspect 42, wherein exposing the host cells to the micro-current increases production of at least one metabolite compared to identical cells that are not exposed to the micro-current.

Aspect 44. The extract of aspect 43, wherein the at least one metabolite comprises a coenzyme.

Aspect 45. The extract of aspect 44, wherein the coenzyme comprises NAD, NADP, NADH, NADPH, or any combination thereof.

Aspect 46. The extract of any one of aspects 28-45, wherein the host cells are exposed the micro-current for from about 30 minutes to about 72 hours.

Aspect 47. A nutritional composition comprising the extract according to any one of aspects 26-46.

Aspect 48. The nutritional composition of aspect 47, formulated as an oral dosage form.

Aspect 49. The nutritional composition of aspect 48, wherein the oral dosage form comprises a capsule, a tablet, a sublingual strip, a buccal strip, a troche, a functional food, a powder, a pellet, or a beverage.

Aspect 50. The nutritional composition of any one of aspects 47-49, further comprising at least one excipient.

Aspect 51. The nutritional composition of aspect 50, wherein the excipient comprises a sweetener, a flavoring agent, a coloring agent, a stabilizer, a thickener, or any combination thereof.

Aspect 52. The nutritional composition of aspect 51, wherein the sweetener comprises saccharin, cyclamate, aspartame, steviol glycosides, or any combination thereof.

Aspect 53. The nutritional composition of aspect 51 or 52, wherein the stabilizer comprises ethanol, n-propanol, glycerol, a polyethylene glycols with a molecular weights between 200 Da and 600 Da, diethylene glycol monoethyl ether, 1,2-propylene glycol, urea, urethane, acetamide, N-methyl acetamide, N,N-diethyl acetamide, N,N-dimethyl acetamide, ethylenediamine, hydroxyethyl theophylline, tromethamine, an aliphatic amino acid, or any combination thereof.

Aspect 54. The nutritional composition of any one of aspects 51-53, wherein the thickener comprises corn starch, xanthan gum, gelatin, pectin, potato starch, tapioca starch, arrowroot, agar-agar, or any combination thereof.

Aspect 55. The nutritional composition of any one of aspects 51-54, wherein the nutritional composition is a sublingual strip or a buccal strip and wherein the at least one excipient comprises a permeation enhancer, a disintegrant, a saliva stimulating agent, a strip-forming polymer, a plasticizer, or any combination thereof.

Aspect 56. The nutritional composition of aspect 55, wherein the strip-forming polymer comprises hydroxypropyl methylcellulose (HPMC) E3, HPMC E5, HPMC E15, HPMC K-3, methylcellulose A-3, methylcellulose A-6, methylcellulose A-15, pullulan, carboxmethylcellulose or a derivative thereof, polyvinylpyrollidone (PVP) K-90, pectin, gelatin. sodium alginate, hydroxypropylcellulose, polyvinyl alcohol, maltodextrins, calcium alginate, a polyactive carbohydrate, chitosan, or any combination thereof.

Aspect 57. The nutritional composition of aspect 55 or 56, wherein the permeation enhancer comprises 2,3-lauryl ether, aprotinin, azone, benzalkonium chloride, cetylpyridinium chloride, cetyltrimethyl ammonium bromide, cyclodextrin, dextran sulfate, lauric acid, lysophosphatidylcholine, menthol, phosphatidylcholine, polyoxyethylene, polysorbate 80, propylene glycol, disodium ethylenediaminetetraacetic acid (EDTA), sodium glycocholate, sodium glycodeoxycholate, sodium lauryl sulfate, sodium salicylate, sodium taurocholate, sodium taurodeoxycholate, or any combination thereof.

Aspect 58. The nutritional composition of any one of aspects 55-57, wherein the saliva stimulating agent comprises citric acid, malic acid, lactic acid, ascorbic acid, or any combination thereof.

Aspect 59. The nutritional composition of any one of aspects 55-58, wherein the disintegrant comprises cross-linked polyvinyl pyrrolidone, sodium carboxymethyl starch, sodium carboxymethyl cellulose, microcrystalline cellulose, or any combination thereof.

Aspect 60. The nutritional composition of any one of aspects 55-59, wherein the plasticizer comprises glycerol, dibutyl phthalate, polyethylene glycol, or any combination thereof.

Aspect 61. The nutritional composition of any one of aspects 47-60, formulated as a powder or liquid additive to be mixed with food or a beverage.

Aspect 62. The nutritional composition of any one of aspects 47-60, formulated as hydrogel microparticles.

Aspect 63. The nutritional composition of aspect 62, wherein the hydrogel microparticles comprise chitosan, acetic acid, sodium alginate, calcium alginate, a polyactive carbohydrate, glucosamine, chondroitin, or any combination thereof.

Aspect 64. The nutritional composition of any one of aspects 47-63, wherein the nutritional composition is formulated as a food additive, a food seasoning, a flavor enhancer, or a component thereof.

Aspect 65. The nutritional composition of any one of aspects 47-64, wherein the nutritional composition can be stored at room temperature.

Aspect 66. The nutritional composition of any one of aspects 47-64, wherein the nutritional composition is refrigerated or frozen prior to use.

Aspect 67. A pharmaceutical composition comprising the extract of any one of aspects 26-46.

Aspect 68. The pharmaceutical composition of aspect 67, wherein the pharmaceutical composition is formulated for sublingual, topical, oral, intravenous, injectable, subcutaneous, intranasal, inhalable, rectal, vaginal, intratympanic, intracochlear, or ocular administration.

Aspect 69. The pharmaceutical composition of aspect 68, wherein a formulation for oral administration comprises a sublingual strip, a buccal strip, a mouth rinse, a mouthwash, a toothpaste, a coating for the teeth, a tablet, a capsule, a candy, a troche, a powder, a pellet, a beverage, gum, a chewable tablet, a gummy candy, or any combination thereof.

Aspect 70. The pharmaceutical composition of aspect 68, wherein a formulation for topical administration comprises a cream, ointment, lotion, spray, soap, nail treatment, body wrap, face mask, swab, lip balm, transdermal patch, scalp treatment, aftershave lotion, or any combination thereof.

Aspect 71. The pharmaceutical composition of aspect 70, wherein the formulation for topical administration can be applied to the skin, scalp, fingernails, toenails, hair, mucous membranes, or any combination thereof.

Aspect 72. The pharmaceutical composition of aspect 68, wherein a formulation for ocular administration comprises eye drops, ointment, or gel.

Aspect 73. The pharmaceutical composition of aspect 68, wherein a formulation for rectal delivery comprises a suppository, enema, or cream.

Aspect 74. The pharmaceutical composition of aspect 68, wherein a formulation for vaginal delivery comprises a suppository or cream.

Aspect 75. The pharmaceutical composition of aspect 68, wherein a formulation for nasal or inhalable delivery comprises a swab, nasal spray, nebulizer, metered dose inhaler, or any combination thereof.

Aspect 76. The pharmaceutical composition of any one of aspects 68-75, wherein the pharmaceutical composition is available by prescription or over the counter.

Aspect 77. The pharmaceutical composition of any one of aspects 68-76, wherein the pharmaceutical composition comprises one or more of an anti-inflammatory property, an anti-itch property, a hair growth property, or any combination thereof.

Aspect 78. The pharmaceutical composition of any one of aspects 68-77, wherein the pharmaceutical composition can be stored at room temperature.

Aspect 79. The pharmaceutical composition of any one of aspects 68-77, wherein the pharmaceutical composition is refrigerated or frozen prior to use.

Aspect 80. A method for providing nutrition to a subject in need thereof, the method comprising administering the nutritional composition of any one of aspects 47-66 or the pharmaceutical composition of any one of aspects 67-79 to the subject.

Aspect 81. The method of aspect 80, wherein the subject is a mammal or a bird.

Aspect 82. The method of aspect 81, wherein the mammal is a human, non-human primate, cat, dog, rat, mouse, hamster, guinea pig, rabbit, horse, cattle, swine, goat, or sheep.

Aspect 83. The method of aspect 81, wherein the bird is a chicken, turkey, duck, or parrot.

Aspect 84. A cosmetic composition comprising the extract of any one of aspects 26-46 and a physiologically acceptable medium.

Aspect 85. The cosmetic composition of aspect 84, wherein the cosmetic composition comprises a tooth whitener, lotion, hair conditioner, soap, deodorant, aftershave lotion, shaving cream, lip balm, face mask, scalp treatment, or a combination thereof.

Aspect 86. A plant grown by the process comprising contacting plant gamete cells, a plant reproductive organ, or a plant callus with the extract of any one of aspects 26-46.

Aspect 87. The plant of aspect 86, wherein the plant is produced by a method comprising the steps of:
(a) contacting a plant callus with the extract;
(b) culturing the plant callus; and
(c) growing the plant from the plant callus.

Aspect 88. A method for enhancing at least one physiological property of a plant, the method comprising applying the extract of any one of aspects 26-46 to the plant.

Aspect 89. The method of aspect 88, wherein the at least one physiological property comprises leaf area, root length, growth rate, immunity to pathogens, stress tolerance, fruit size, fruit number, production of a plant metabolite, or any combination thereof.

Aspect 90. A method for increasing growth of cells in a microbial culture, the method comprising contacting the microbial culture with a composition comprising the extract of any one of aspects 26-46.

Aspect 91. The method of aspect 90, wherein performing the method increases production of one or more desired metabolites from the microbial culture.

Aspect 92. The method of aspect 90 or 91, wherein performing the method increases a count of microbial cells or colonies in a culture dish or vessel.

Aspect 93. The method of any one of aspects 90-92, wherein the microbial culture comprises a bacterial culture or a fungal culture.

Aspect 94. The method of aspect 93, wherein the fungal culture comprises a yeast culture.

Aspect 95. The method of aspect 94, wherein the yeast culture comprises Saccharomyces cerevisiae, Yarrowia lipolytica, Scheffersomyces stipitis, Kluyveromyces lactis, Dekkera bruxellesis, a Candida species, Monascus purpureus, or any combination thereof.

Aspect 96. The method of aspect 93, wherein the bacterial culture comprises E. coli, a Lactobacillus species, Pyrococcus furiosus, Thermus aquaticus, Bacillus stearothermophilus, Thermus filiformis, Thermus thermophilus, a Xanthomonas species, Aspergillus niger, a Trichoderma species, Mucor miehei, a Mortierella species, a Clostridium species. a Streptomyces species, a Penicillium species, Acetobacter aceti, a Bacillus species, a Klebsiella species, Trichoderma polysporum, or any combination thereof.

Aspect 97. The method of any one of aspects 91-96, wherein the one or more desired metabolites comprise invertase, raffinase, lactase, lipase, lactic acid, insulin, a heat stable DNA polymerase, xanthan gum, citric acid, aminoacylase, glucoamylase, catalase, glucose oxidase, pectinase, pectin lyase, protease, cellulose, botulinum toxin, butanol, chloramphenicol, daptomycin, fosfomycin, lincomycin, neomycin, nourseothricin, puromycin, streptomycin, tetracycline, oleandomycin, tunicamycin, mycangimycin, boromycin, bambermycin, clavulanic acid, guadinomine, ivermectin, migrastatin, bleomycin, erythromycin, geldanamycin, penicillin and other beta-lactam antibiotics, precursors to semi-synthetic beta-lactam antibiotics, acetic acid; α-amylase, β-amylase, glucose isomerase, penicillin amidase, asparaginase, pullulanase, cyclosporine A, statin drugs, insecticides, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Preparation of DNA Constructs

The DNA construct was composed of the genetic components described herein and assembled in plasmid vectors (e.g., pYES2). Sequences of genes and/or proteins with desired properties were identified in GenBank; these included a gene that encodes casein, a gene that encodes an ovalbumin fragment, a gene that encodes lactalbumin, and a gene that encodes GMP. These sequences were synthesized by CloneTex Systems, Inc. (Austin, TX). Other genetic parts were also obtained for inclusion in the DNA constructs including, for example, promoter genes (e.g., GAL1 promoter), reporter genes (e.g., enhanced green fluorescent reporter protein), and terminator sequences (e.g., CYC1 terminator). These genetic parts included restriction sites for ease of insertion into plasmid vectors.

The cloning of the DNA construct into the biological devices was performed as follows. Sequences of individual genes were amplified by polymerase chain reaction using primers that incorporated restriction sites at their 5′ ends to facilitate construction of the full sequence to be inserted into the plasmid. Genes were then ligated using standard protocols to form an insert. The plasmid was then digested with restriction enzymes according to directions and using reagents provided by the enzymes' supplier (Promega). The complete insert, containing restriction sites on each end, was then ligated into the plasmid. Successful construction of the insert and ligation of the insert into the plasmid were confirmed by gel electrophoresis.

In some experiments, each gene was PCR amplified using gene-specific overlap primers and assembled sequences were sub-cloned into a pYES2 vector. PCR amplified pieces of all fragments were combined using homologous recombination technology (Gibson Assembly). Clones obtained after transformation were sequenced and analyzed for DNA sequence accuracy.

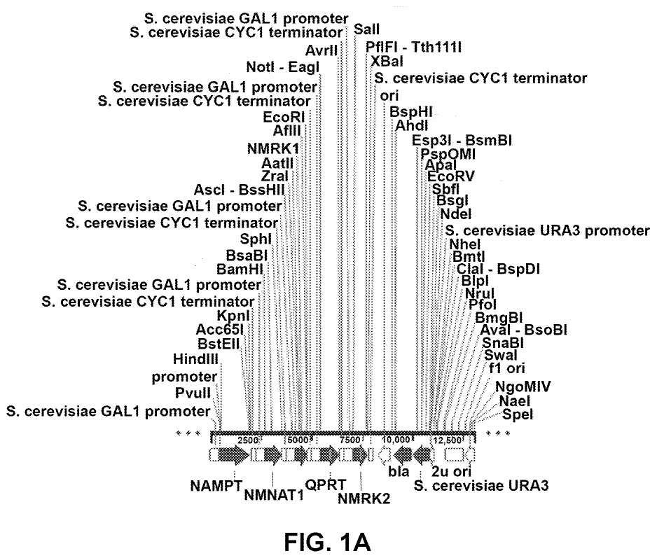

From 5′ to 3′, one version of the construct for making a NAD producing DNA composition or extract includes (a) a gene that encodes NAMPT; (b) a gene that encodes NMNAT; (c) a gene that encodes NRK1; (d) a gene that encodes NRK2; and (e) a gene that encodes QPRT (FIGS. 1A-1B).

PCR was used to enhance DNA concentration using a Mastercycler Personal 5332 ThermoCycler (Eppendorf North America) with specific sequence primers and the standard method for amplification (Sambrook, J., E. F. Fritsch, and T. Maniatis, 1989, Molecular Cloning: A Laboratory Manual, 2nd ed., Vol. 1, Cold Spring Harbor Laboratory Press: Cold Spring Harbor, NY). Digestion and ligation were used to ensure assembly of DNA synthesized parts using restriction enzymes and reagents (PCR master mix of restriction enzymes: XhoI, KpnI, XbaI, EcoRI, BamHI, and HindIII, with alkaline phosphatase and quick ligation kit, all from Promega). DNA was quantified using a NanoVue spectrophotometer (GE Life Sciences) and a standard UV/Visible spectrophotometer using the ratio of absorbances at 260 nm and 280 nm. In order to verify final ligations, DNA was visualized and purified via electrophoresis using a Thermo EC-150 power supply.

The DNA construct was made with gene parts fundamental for expression of sequences such as, for example, native and constitutive promoters, reporter genes, and transcriptional terminators or stops. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the method described below.

### Example 2: Selection of Microorganisms and Development of Competent Cells

Some constructs were produced using transfected yeasts (Saccharomyces cerevisiae, ATCC® 200892™). Yeast cells were made competent by subjecting them to an electrochemical process adapted from Gietz and Schiestl (Nature Protocols, 2007, 2:35-37). Briefly, a single yeast colony was inoculated into 100 mL YPD (yeast extract peptone dextrose) growth media. Yeast was grown overnight on a shaker at 30° C. to OD₆₀₀=1.0. (Acceptable results were obtained with OD₆₀₀ values ranging from 0.6 to 1.8.) Cells were centrifuged at 2000 rpm in a tabletop centrifuge and resuspended in 10 mL TEL buffer (10 mM Tris-HCl, 1 mM EDTA, 0.1 M LiAc, PH=7.5) and shaken vigorously overnight at room temperature. Alternatively, INVSc1 cells were prepared to be competent using a kit from Sigma-Aldrich, Inc. Cells were again centrifuged and resuspended in 1 mL TEL buffer. Cells prepared in this manner could be stored in the refrigerator for up to one month.

### Example 3: Transformation of Microbial Cells

Competent cells were stored in the freezer until needed. Cells were thawed on ice and 100 μL of competent cells in TEL buffer were placed in a sterile 1.5 mL microcentrifuge tube. To this was added 5 μL of a 10 mg/mL solution of salmon sperm DNA (carrier DNA). Transforming DNA was added in various amounts. From 1 to 5 μg was sufficient for plasmids from commercial sources, but more DNA was required when transforming yeast with artificial DNA constructs. 10 μL of the DNA device were added to the microcentrifuge tube containing the competent yeast cells and the contents of the tube were mixed. The DNA-yeast suspension was incubated for 30 min at room temperature.

A PLATE solution (consisting of 40% PEG-3350 in 1×TEL buffer) was prepared. 0.7 mL of PLATE solution was added to the DNA-yeast suspension and the contents were mixed thoroughly and incubated for 1 h at room temperature. The mixture was placed in an electromagnetic chamber for 30 minutes. Cells were then heated at 42° C. for 5-10 minutes and 250 μL aliquots were plated on yeast malt agar to which selective growth compounds had been added. Plates were incubated overnight at 30° C.

DNA expression and effectiveness of transformation were determined by fluorescence of the transformed cells expressed in fluorescence units (FSUs) using a 20/20 Luminometer (Promega) according to a protocol provided by the manufacturer. Plasmid DNA extraction, purification, PCR, and gel electrophoresis were also used to confirm transformation. Different transformed devices were obtained. Different types of fluorescent reporter proteins were used (e.g., yellow, red, green, and cyan) for all transformed cells and/or constructs. However, the yellow fluorescent protein was preferred. When no fluorescent reporter protein was assembled, no fluorescence was observed.

S. cerevisiae cells were subjected to transformation with the modified pYES2 plasmids for producing metal- and contaminant-binding components as described above. Transformed yeast cells were incubated for 30 min at 28-30° C. Colonies of transformed yeast cells were selected, their DNA isolated and subjected to PCR amplification. Two control treatments were also carried out: (1) a negative control involving competent yeast and nuclease free water instead of a plasmid and (2) a positive control involving competent yeast with unmodified pYES2 plasmid.

Four clones were selected from a transformed plate and processed for full-length DNA sequencing. A clone with 100% DNA sequence accuracy was selected for further processing and was used to obtain a high concentration of plasmid construct at a mid-scale plasmid purification level. Yeast competent cells were transformed with the recombinant plasmid and selected on synthetic complete (SC) dropout plate deficient in uracil. Well isolated clones were isolated and preserved in YPD medium containing 15% glycerol for storage at −80° C.

### Example 4: Production of Microbial Extracts and NAD+ Metabolites

Microbial Extracts Containing NAD+ Metabolites

The following non-limiting procedure was used to produce the disclosed extracts:

### Method

(a) Yeast transformed with the device depicted in FIGS. 1A-1B were fermented at 37° C. for 48 hours, where culture was conducted with 25 mL of device inoculum in 1 L Luria broth and having 1 μg/mL ampicillin and 100 μM isopropyl-β-D-thiogalactopyranoside (IPTG).
(b) The culture was sterilized by autoclaving at 121° C. for 30 minutes and then centrifuged.
(c) The mixture was filtered with a 0.45 μm filter to produce a supernatant composed of the desired extract.

The NAD device, which was cloned in yeast, will be grown in the specific media with or without micro-current within a time-course process for at least a week. This lysate will be tested in various oral dosage forms, including sublingually and as beverage, as well as others.

In some experiments, the disclosed devices were grown on yeast malt broth at 30° C. for 4 days with shaking at 150 rpm with or without microcurrent (300 mV). The cultures were treated with 40 nM precursor β-NMN after 48 h of growth. Cultures of non-transformed Saccharomyces cerevisiae were also grown as a control. Aliquots were removed every 24 hours to measure optical density, pH, redox, cell count, and obtain an extract for every sample.

A protocol was followed to find out the NAD concentration of the cultures. 50 mL of the culture were centrifuged for 5 mins at 4° C. and 10,000 g. The pellet was preserved and then washed with cold PBS repeating the procedure just mentioned for two to three times. Then, 40 μL of the pellet were saved to be later analyzed for NAD concentration.

In some cases, an alternative growth medium was used. In these experiments, a disclosed device was grown on yeast malt broth (YMB) overnight at 30° C. Then, media of 100×molasses+1 g/L of YMB was prepared for culture growth. This media was inoculated, and the culture was grown for 4 days with agitation of 150 rpm in a shaking incubator under 300 mV of current. The culture was induced with 2% raffinose and 1% galactose. Samples were taken every 24 hours to measure optical density, pH, redox, cell count, and obtain an extract of every sample.

Supplementary nutritional value can be added to the NAD formulation including, but not limited to, vitamins A and C. This addition will make the NAD more nutritional with better benefit for the consumer since these vitamins serve as antioxidants and also contribute to overall energy levels. For this purpose, the NAD lysate will be mixed with a lysate from one or more additional biological devices producing other nutrients at different ratios. In some experiments, the composition also contains stevia or an extract or lysate from a stevia-producing device, which would improve the taste of the nutritional composition.

Exemplary biological devices to produce stevia (steviol glycosides) are described in U.S. Pat. No. 11,365,417 and US Patent Application Publication 2023/0212588. Exemplary biological devices to produce carotenoids are described in U.S. Pat. No. 11,603,549. Genes from steviol glycoside devices and carotenoid devices can also be combined into one device to streamline processing.

Once the extracts and/or lysates are produced, they can be processed into oral dosage forms as described herein. In some aspects, lysates from the devices described herein are mixed in any proportion (e.g. 95:1, 5:1, 2:1, 1:1, etc.).

### Example 5: Hydrogel/Microparticle Encapsulation

Hydrogel beads were produced with chitosan (1% w/v) in acetic acid 3% (w/v) and sodium alginate (2% w/v). Compounds to produce the hydrogels were sodium hydroxide (NaOH) and calcium chloride (CaCl₂)), respectively. Extracts and lysates from the disclosed devices were homogenized in 1.5 mL microcentrifuge tubes. Furthermore, chitosan and sodium alginate were added to the final mixture and hydrogel beads were obtained. Proportions used to produce hydrogels with the disclosed NAD-containing lysates and extracts varied. In one experiment, 900 μL of chitosan/sodium alginate solution were used with 300 μL of NAD-containing lysate or extract.

### Example 6: Testing of the Disclosed Devices

Lysates and extracts from the NAD devices will be subjected to NAD analysis via known experimental methods to verify production of the disclosed devices as well as nutritional value and/or toxicity.

10 replicates from cultures produced as described in Example 4 were quantitatively analyzed for determination of NAD, NADH, and nicotinamide using LC-MRM/MS. 9 serially diluted calibration solutions of NAD, NADH, and nicotinamide were prepared by dissolving in an internal standard solution of isotope-labeled NAD, NADH, and nicotinamide.

An aliquot of each sample was precisely weighed in a microcentrifuge tube. 10 μL of 80% methanol per mg of wet mass or raw material was added to each tube. Metabolites were extracted using two metal beads at 30 Hz for 3 min on an MM 400 mill mixer. After centrifugation, the clear supernatants were diluted 10× and 10,000×, respectively, with an isotopically-labeled internal standard solution. The samples were then used to perform an assay as follows:

5 μL aliquots of the samples described above, which contained solvent and concentration matched isotope-labeled NAD, NADH, and nicotinamide as internal standards were injected onto a C18 UPLC column for LC-MRM/MS with positive ion detection on an Agilent 6495B UHPLC instrument with an Agilent 6495 QQQ mass spectrometer. Mobile phase was ammonium acetate buffer and methanol and was used for binary solvent gradient elution.

Linear regression calibration curves for individual metabolites were constructed using the data thus acquired. Concentrations of the metabolites detected in the samples were calculated by interpolating the calibration curves using data acquired from the sample solutions. Results are consistent with the colorimetric assay presented below.

The disclosed devices showed higher production of NAD with or without current compared to untransformed controls. Results using molasses medium suggest this is an economical alternative for growing the devices instead of or in addition to standard culture media.

A colorimetric assay was also performed as follows. A protocol from MSE NAD/NADH Colorimetric Assay Kit was followed to prepare samples for measuring NAD concentration first, and then ultrafiltration 10K was used to remove the enzyme used in the assay.

Preparation of samples included taking 20 μL of the standards solutions at different concentrations as well as sample supernatant, then 120 μL were added to each standard and sample, as well as 40 μL of chromogenic agent. Samples were fully mixed and incubated at 37° C. for 30 min. Duplicates of samples were done to measure NAD total and NADH alone. After samples were incubated at 30° C., samples were measured using a UV/VIS spectrophotometer at 450 nm.

### NADH Detection

Samples were heated at 60° C. using a water bath for 30 min, so the NAD+ of the samples were decomposed and only NADH remained. NADH reduces WST-8 to form yellow product, and the amount of NADH was determined by measuring the OD value at 450 nm.

### NAD+ Detection

The content of NAD+ in each sample can be obtained subtracting the OD of the total content of NAD total and the OD of NADH obtained of the first two steps. Results of the colorimetric assay are shown in Table 6:

TABLE 6
Metabolite Concentration

| | | Protein | | | |
|---|---|---|---|---|---|
| | Wet mass | (μg/mg wet | NAD | NADH | Nicotinamide |
| Sample | (mg) | mass) | (nmol/g) | (nmol/g) | (nmol/g) |
| Disclosed device, no | 64.97 | 8.67 | 10.8 | 5.80 | 1.00 |
| microcurrent | | | | | |
| Disclosed device, | 169.9 | 7.68 | 2.85 | 1.52 | 0.66 |
| microcurrent | | | | | |
| Non-transformed yeast, | 113.33 | 5.51 | 2.25 | 0.29 | 0.26 |
| no microcurrent | | | | | |
| Non-transformed yeast, | 51.95 | 8.41 | 3.43 | 0.57 | 0.33 |
| microcurrent | | | | | |
| Disclosed device, | 58.2 | 7.23 | 6.70 | 0.52 | 0.58 |
| microcurrent, molasses | | | | | |
| growth medium | | | | | |
| Commercially available | 52.66 | 0.57 | 0.01 | 0.01 | 1.27 × 10⁷ |
| NAD | | | | | |

Nanoparticles and hydrogels/microparticles will be subjected to size analysis via known experimental methods.

Production of the devices and extracts will be tested at different temperatures (10, 15, 25, 30, 37, and 45° C.) and pH values (between 3.5 and 7.5) in order to determine optimum conditions for production of the desired metabolites as well as their stability and capacity to be stored long-term.

In some experiments, precursors of NAD can be added to device cultures in order to increase levels of NAD. These include, but are not limited to, nicotinamide mononucleotide (NMN), nicotinic acid (NA), nicotinamide riboside (NR), tryptophan, and related compounds and metabolites. In order to optimize concentration and addition of these precursors, they are introduced in different concentrations and at different times (24, 30, 48, and 60 hours after the inoculation of the cultures). Precursors can be added in a ratio of 1:1, 1:2, 1:3, 3:1, or 2:1 relative to the device concentration.

Throughout this application, various publications are referenced. The disclosures of these publications in their entireties are hereby incorporated by reference into this application in order to more fully describe the compounds, compositions, and methods described herein.

### Example 7: Water Solubility of the NAD+

Solubility is defined as the maximum amount of solute that can be dissolved in a known amount of solvent at a particular temperature.

$$Solubility ( \frac{g}{L} ) = \frac{Mass of solute in Solution ( g )}{Volume of Solvent ( L )}$$

Therefore, the main objective of this procedure to measure the solubility of the disclosed device lysate in water, as compared to a control, which is commercial NAD.

The chosen solvent was water. Then, different solutions were prepared, including at 0.5, 1, 2, 5 mg/mL concentrations, for each sample. Samples were mixed thoroughly for at least 30 minutes. A water bath at 37° C. was also used to observe how temperature affected solubility. Then, the suspensions were allowed to sit for 24 h at room temperature for them to reach equilibrium. Centrifugation was used at 9,000 rpm for 10 min to remove any undissolved material.

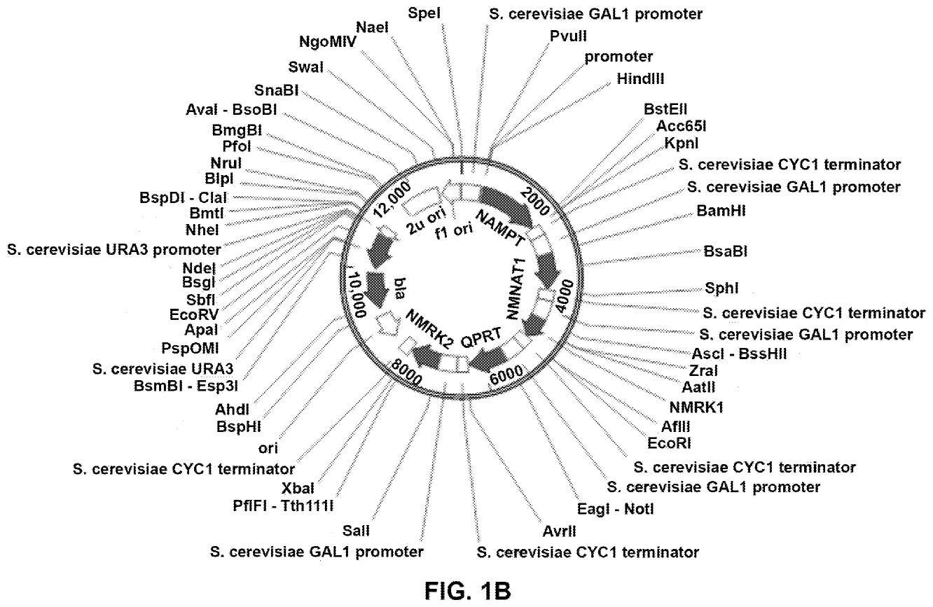

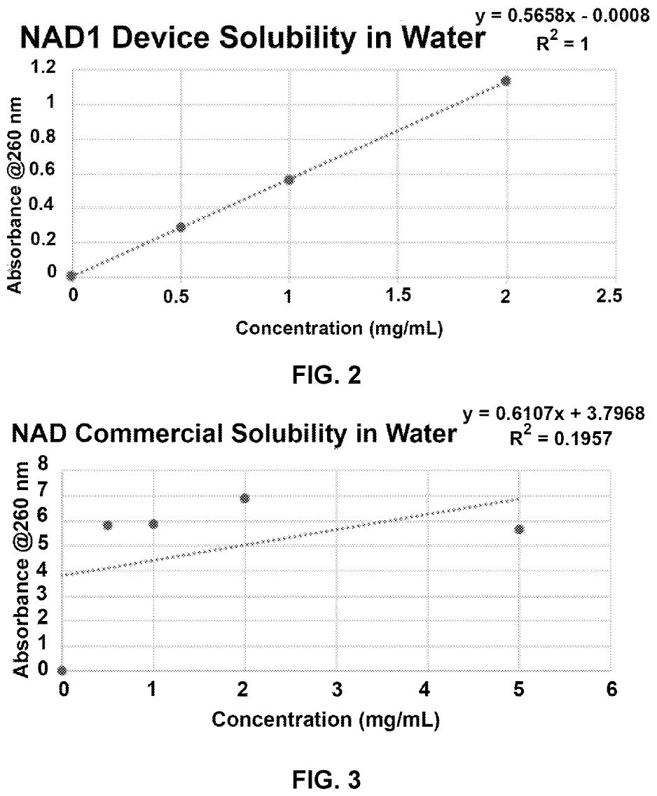

Then, the degree of solubility was measured using a UV-VIS Spectrophotometer at a wavelength of 260 nm to quantify the dissolved compound. A standard curve was plotted to see how the absorbance related to the concentration of product as seen in FIG. 2. A standard curve for commercial NAD is shown in FIG. 3.

### Results

It was found that the control (commercial NAD) did not dissolve completely in water, thus the particles obstructed the absorbance of light and the standard curve was not optimal. However, the disclosed device lysate dissolved completely in water, giving a graph that is directly proportional between the absorbance and the concentration of solute (FIG. 2).

### CONCLUSION

The disclosed device lysate is completely soluble in water, as compared to the control (commercial NAD). Thus, the disclosed lysate is more suitable to be used in biological systems, including humans, animals, plants, and microorganisms.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be exemplary.

What is claimed is:

1. A DNA construct comprising the following genetic components:
(a) a gene that encodes nicotinamide phosphoribosyltransferase (NAMPT);
(b) a gene that encodes nicotinamide mononucleotide adenylytransferase (NMNAT);
(c) a gene that encodes nicotinamide riboside kinase 1 (NRK1);
(d) a gene that encodes nicotinamide riboside kinase 2 (NRK2); and
(e) a gene that encodes quinolinate phosphoribosyltransferase (QPRT).

2. The DNA construct of claim 1, wherein the gene that encodes the NAMPT has SEQ ID NO. 1 or at least 70% homology thereto.

3. The DNA construct of claim 1, wherein the gene that encodes NMNAT has SEQ ID NO. 2 or at least 70% homology thereto.

4. The DNA construct of claim 1, wherein the gene that encodes NRK1 has SEQ ID NO. 3 or at least 70% homology thereto.

5. The DNA construct of claim 1, wherein the gene that encodes NRK2 has SEQ ID NO. 4 or at least 70% homology thereto.

6. The DNA construct of claim 1, wherein the gene that encodes QPRT has SEQ ID NO. 5 or at least 70% homology thereto.

7. The DNA construct of claim 1, wherein the construct further comprises at least one promoter.

8. The DNA construct of claim 7, wherein the at least one promoter is GAL1 promoter, and the GAL1 promoter is positioned before the gene that encodes NAMPT, the gene that encodes NMNAT, the gene that encodes NRK1, the gene that encodes NRK2, the gene that encodes QPRT, or any combination thereof.

9. The DNA construct of claim 1, wherein the construct comprises from 5′ to 3′ the following genetic components in the following order: (a) the gene that encodes NAMPT; (b) the gene that encodes NMNAT; (c) the gene that encodes NRK1; (d) the gene that encodes NRK2; and (e) the gene that encodes QPRT.

10. The DNA construct of claim 1, wherein the construct comprises from 5′ to 3′ the following genetic components in the following order: (a) the gene that encodes NAMPT having SEQ ID NO. 1 or at least 70% homology thereto; (b) the gene that encodes NMNAT having SEQ ID NO. 2 or at least 70% homology thereto; (c) the gene that encodes NRK1 having SEQ ID NO. 3 or at least 70% homology thereto; (d) the gene that encodes NRK2 having SEQ ID NO. 4 or at least 70% homology thereto; and (e) the gene that encodes QPRT having SEQ ID NO. 5 or at least 70% homology thereto.

11. The DNA construct of claim 1, wherein the construct comprises from 5′ to 3′ the following genetic components in the following order: (a) the gene that encodes NAMPT, (b) a CYC1 terminator, (c) a GAL1 promoter, (d) the gene that encodes NMNAT, (e) a CYC1 terminator, (f) a GAL1 promoter, (g) the gene that encodes NRK1, (h) a CYC1 terminator, (i) a GAL1 promoter, (j) the gene that encodes NRK2; (k) a CYC1 terminator; (l) a GAL1 promoter; and (m) the gene that encodes QPRT.

12. The DNA construct of claim 1, wherein the construct comprises from 5′ to 3′ the following genetic components in the following order: (a) the gene that encodes NAMPT having SEQ ID NO. 1 or at least 70% homology thereto, (b) a CYC1 terminator, (c) a GAL1 promoter, (d) the gene that encodes NMNAT having SEQ ID NO. 2 or at least 70% homology thereto, (e) a CYC1 terminator, (f) a GAL1 promoter, (g) the gene that encodes NRK1 having SEQ ID NO. 3 or at least 70% homology thereto, (h) a CYC1 terminator, (i) a GAL1 promoter, (j) the gene that encodes NRK2 having SEQ ID NO. 4 or at least 70% homology thereto; (k) a CYC1 terminator; (l) a GAL1 promoter; and (m) the gene that encodes QPRT having SEQ ID NO. 5 or at least 70% homology thereto.

13. The DNA construct of claim 1, wherein the DNA construct has SEQ ID NO. 6.

14. A vector comprising the DNA construct of claim 1.

15. The vector of claim 14, wherein the vector is a plasmid.

16. The vector of claim 15, wherein the plasmid is pWLneo, pSV2cat, pOG44, pXT1, pSG, pSVK3, pBSK, pBSKII, pYES, pYES2, pET, pBAD, pUC, or pUC19.

17. A biological device comprising host cells transformed with the DNA construct of claim 1.

18. The biological device of claim 17, wherein the host cells comprise Saccharomyces cerevisiae.

19. An extract produced by culturing the biological device of claim 17 in a culture medium, wherein the extract comprises NAD, NADH, NADP, NADPH, or any combination thereof.

20. The extract of claim 19, wherein the host cells are lysed to produce a lysate.
